(19) United States
(12) Patent Application Publication
(10) Pub. No.: US 2024/0358679 A1
(43) Pub. Date: Oct. 31, 2024

(54) PHARMACEUTICAL AND FOOD COMPOSITIONS FOR THE PREVENTION, ALLEVIATION, OR TREATMENT OF INFLAMMATORY DISEASES COMPRISING 1-ALKYL-5-ARYLIDENE-2-SELENOXOIMIDAZOLIDIN-4-ONE AND ITS DERIVATIVES

(71) Applicants: DUKSUNG WOMEN'S UNIVERSITY INDUSTRY-ACADEMIC COOPERATION FOUNDATION, Seoul (KR); INDUSTRY ACADEMIC COOPERATION FOUNDATION OF YEUNGNAM UNIVERSITY, Gyeongsan-si Gyeongsangbuk-do (KR)

(72) Inventors: Sang Hyup LEE, Seoul (KR); Dong Young CHOI, Gyeongsan-si Gyeongsangbuk-do (KR); Hyun Sung CHO, Daejeon (KR); Cho Rong KIM, Seoul (KR); Yoo Jin LIM, Seoul (KR); Ye Eun KIM, Seoul (KR); Yu Ra JEONG, Seoul (KR)

(21) Appl. No.: 18/768,902

(22) Filed: Jul. 10, 2024

Related U.S. Application Data
(63) Continuation of application No. PCT/KR2023/000277, filed on Jan. 6, 2023.

(30) Foreign Application Priority Data
Jan. 10, 2022 (KR) 10-2022-0003286

(51) Int. Cl. A61K 31/4152; A61K 31/4155; A61K 31/4439; A61K 31/4525; A61K 31/496; A61K 31/5377; A61P 29/00

(52) U.S. Cl. CPC A61K 31/4152; A61K 31/4155; A61K 31/4439; A61K 31/4525; A61K 31/496; A61K 31/5377; A61P 29/00

(57) ABSTRACT

The present invention relates to a composition for the prevention, alleviation, or treatment of inflammatory diseases, comprising 1-alkyl-5-arylidene-2-selenoxoimidazolidin-4-one and derivatives thereof. Specifically, the novel compounds of the present invention, 1-alkyl-5-arylidene-2-selenoxoimidazolidin-4-one and derivatives thereof, can be used as excellent pharmaceutical compositions and food compositions for the prevention, alleviation, or treatment of inflammatory diseases.

### CROSS-REFERENCE TO RELATED APPLICATIONS

This application is a continuation of International application PCT/KR2023/000277 filed 6 Jan. 2023, which claims the benefit of, and relies on the filing date of, Korean Patent Application No. 10-2022-0003286 filed 10 Jan. 2022, the entire disclosure of which is incorporated herein by reference.

### TECHNICAL FIELD

The present invention relates to compositions for the prevention, alleviation, or treatment of inflammatory diseases comprising 1-alkyl-5-arylidene-2-selenoxoimidazolidin-4-one and its derivatives. Specifically, the novel compounds of the present invention, 1-alkyl-5-arylidene-2-selenoxoimidazolidin-4-one and its derivatives, can be used as effective compositions for the prevention, alleviation, or treatment of inflammatory diseases.

### BACKGROUND ART

Reactive oxygen species, known to increase in the body due to exposure to ultraviolet radiation, ionizing radiation, alcohol and tobacco, excessive stress, pollutants, overeating, and strenuous exercise, are perceived as a necessary evil within the body, contributing to aging, diabetes, allergic diseases, and various other conditions. To reduce reactive oxygen species levels in the body, an increasing number of individuals are consuming dietary supplements or foods containing antioxidants such as vitamins A, C, E, beta-carotene, lutein, and selenium.

Reactive oxygen species are chemically reactive molecules containing oxygen, encompassing terms such as superoxide anion (O₂—), hydrogen peroxide (H₂O₂), hydroxyl radical (OH), peroxynitrite (NO₃—), and hypochlorite ion (OCl—). When phagocytes, including neutrophils, are activated by invading microorganisms, the nicotinamide adenine dinucleotide phosphate oxidase (NOX) complex, which is dispersed in the cell membrane and cytoplasm, assembles to produce reactive oxygen species. These reactive oxygen species play a role in protecting the host from invading microorganisms. However, this process is known to cause host tissue damage and induce inflammation observed in various diseases.

The NOX complex consists of five subunit proteins, gp91^{phox}, p22^{phox}, p47^{phox}, p67^{phox}, p40^{phox} which are normally separate but assemble upon stimulation to produce reactive oxygen species. Reactive oxygen species generated by the NOX complex in phagocytes can induce local inflammation and, due to their high reactivity, may also contribute to cancer development. Reactive oxygen species are implicated as a mechanism for cancer initiation in tissues under chronic inflammatory conditions. The high reactivity of reactive oxygen species can lead to tissue damage, inflammation, and in some cases, cancer, by modifying various substances such as proteins, lipids, carbohydrates, and DNA in tissues.

Several NOX inhibitors have been developed, but it has been reported that there are no drugs specific to any one of the seven isoforms: NOX1, NOX2, NOX3, NOX4, NOX5, DUOX1, and DUOX2. Various isoforms of NOX show differences in expression levels depending on the tissue, leading to ongoing research into the selectivity of each isoform. Overall, inhibiting the generation of reactive oxygen species appears to be the common strategy.

Accordingly, the present inventors have completed the present invention by inhibiting the generation of reactive oxygen species through NOX enzyme inhibition for the fundamental treatment of inflammatory diseases and confirming the therapeutic effect of treating inflammatory diseases by developing novel compounds exhibiting anti-inflammatory effects.

### PRIOR ART DOCUMENTS

### Patent Documents

Korean Registered Patent No. 10-2191491

### Non-Patent Literature

J. P. Taylor and H. M. Tse. The role of NADPH oxidases in infectious and inflammatory diseases. Redox Biology 48 (2021) 102159
Jong Youl Kim, et al. NOX Inhibitors—A Promising Avenue for Ischemic Stroke. Exp Neurobiol. 2017 August; 26(4):195-205
G Aviello and U G Knaus. NADPH oxidases and ROS signaling in the gastrointestinal tract. Mucosal Immunology (2018) 11:1011-1023

### DETAILED DESCRIPTION OF THE INVENTION

### Technical Problem

The object of the present invention is to provide 1-alkyl-5-arylidene-2-selenoxoimidazolidin-4-one and its derivatives, which have the effect of inhibiting NOX enzymes that generate reactive oxygen species and induce inflammation, and to provide a method for manufacturing the same.

Another object of the present invention is to provide a pharmaceutical composition for the prevention, alleviation, or treatment of inflammatory diseases comprising 1-alkyl-5-arylidene-2-selenoxoimidazolidin-4-one and its derivatives, or a pharmacologically acceptable salt thereof, as an active ingredient.

### Solution to Problem

The present invention provides a compound of the following Chemical Formula 1, or a pharmaceutically acceptable salt thereof. Furthermore, the present invention provides a pharmaceutical composition for the prevention or treatment of inflammatory diseases comprising a compound of the following Chemical Formula 1 or a pharmaceutically acceptable salt thereof; a method for the prevention or treatment of inflammatory diseases comprising administering to a subject a compound of the following Chemical Formula 1 or a pharmaceutically acceptable salt thereof; a pharmaceutical composition comprising a compound of the following Chemical Formula 1 or a pharmaceutically acceptable salt thereof for use in the prevention or treatment of inflammatory diseases; and the use of a compound of the following Chemical Formula 1 or a pharmaceutically acceptable salt thereof for the manufacture of a medicament for the prevention or treatment of inflammatory diseases.

The present invention also provides a health functional food composition for the prevention or improvement of inflammatory diseases comprising a compound of the following Chemical Formula 1 or a pharmaceutically acceptable salt thereof; a health functional food composition comprising a compound of the following Chemical Formula 1 or a pharmaceutically acceptable salt thereof for use in the prevention or improvement of inflammatory diseases; and the use of a compound of the following Chemical Formula 1 or a pharmaceutically acceptable salt thereof for the manufacture of a health functional food composition for the prevention or improvement of inflammatory diseases.

The present invention also provides a food composition for the prevention or improvement of inflammatory diseases comprising a compound of the following Chemical Formula 1 or a pharmaceutically acceptable salt thereof; a food composition comprising a compound of the following Chemical Formula 1 or a pharmaceutically acceptable salt thereof for use in the prevention or improvement of inflammatory diseases; and the use of a compound of the following Chemical Formula 1 or a pharmaceutically acceptable salt thereof for the manufacture of a food composition for the prevention or improvement of inflammatory diseases.

Additionally, the present invention provides an anti-inflammatory composition comprising a compound of the following Chemical Formula 1 or a pharmaceutically acceptable salt thereof; a composition comprising a compound of the following Chemical Formula 1 or a pharmaceutically acceptable salt thereof for use in anti-inflammation; and the use of a compound of the following Chemical Formula 1 or a pharmaceutically acceptable salt thereof for the manufacture of an anti-inflammatory agent.

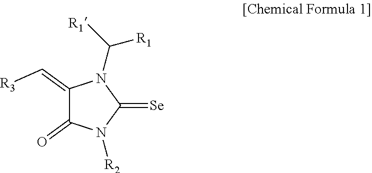

In the above formula,
R₁ and R₁′ are each independently selected from the group consisting of hydrogen, alkyl, cycloalkyl, heterocycloalkyl, aryl, heteroaryl, benzyl, and phenethyl, or R₁ and R₁′ may combine together to form a ring;
R₂ and R₃ are each independently halo, cyano, alkyl, alkoxy, alkenyl, alkynyl, cycloalkyl, heterocycloalkyl, heterocycloalkyl-alkyl, aryl, aryl-alkyl, or heteroaryl;
wherein the alkyl, alkoxy, alkenyl, alkynyl, cycloalkyl, heterocycloalkyl, heterocycloalkyl-alkyl, aryl, aryl-alkyl, or heteroaryl is optionally substituted or unsubstituted with one or more groups selected from hydroxy; halogen; alkyl; -alkyl-hydroxy; -heterocycloalkyl-alkyl-hydroxy; —NH-alkyl-O-alkyl-hydroxy; —NH-alkyl-O-alkyl-halogen; —NH-alkyl-heterocycloalkyl; alkoxy; amino; dialkylamino; nitro; cyano; carbonyl; cycloalkyl; heterocycloalkyl substituted or unsubstituted with alkyl; aryl; and heteroaryl.

In one embodiment, R₁ and R₁′ are each independently selected from the group consisting of hydrogen, C₁-C₆ alkyl, C₃-C₈ cycloalkyl, C₃-C₈ heterocycloalkyl, C₆-C₁₀ aryl, C₅-C₁₀ heteroaryl, benzyl, and phenethyl, or R₁ and R₁′ may combine together to form a ring.

In one embodiment, the above

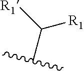

may be

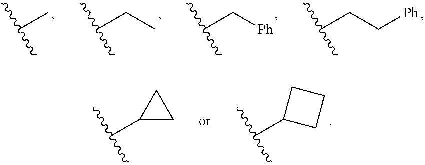

In one embodiment, R₂ may be a C₁-C₁₂ alkyl substituted or unsubstituted with halogen; C₃-C₁₀ cycloalkyl; —C₁-C₆ alkyl-C₃-C₁₀ heterocycloalkyl containing one or more heteroatoms selected from the group consisting of N, S, and O; —C₆-C₁₀ aryl substituted or unsubstituted with halogen or C₁-C₁₂ alkyl; or —C₁-C₆ alkyl-C₆-C₁₀ aryl.

In one embodiment, R₂ may be C₁-C₁₂ alkyl; halogen-substituted C₁-C₆ alkyl; C₃-C₈ cycloalkyl; —C₁-C₆ alkyl-C₃-C₁₀ heterocycloalkyl containing N and O heteroatoms; —C₆-C₁₀ aryl substituted or unsubstituted with halogen or C₁-C₆ alkyl; or —C₁-C₆ alkyl-C₆-C₁₀ aryl.

In one embodiment, R₂ may be C₁-C₁₂ alkyl; C₁-C₆ alkyl substituted with halogen; C₃-C₈ cycloalkyl; —C₁-C₆ alkyl-morpholine; phenyl unsubstituted or substituted with halogen or C₁-C₆ alkyl; or —C₁-C₆ alkyl-phenyl.

In one embodiment, R₃ is C₆-C₁₀ heterocycloalkyl, C₆-C₁₀ aryl or C₆-C₁₀ heteroaryl, and the heterocycloalkyl, aryl or heteroaryl is substituted or unsubstituted with one or more groups selected from the group consisting of hydroxy; halogen; C₁-C₆ alkyl; —C₁-C₆ alkyl-hydroxy; —C₃-C₈ heterocycloalkyl-C₁-C₆ alkyl-hydroxy; —NHC₁-C₆ alkyl-O—C₁-C₆ alkyl-hydroxy; —NHC₁-C₆ alkyl-O—C₁-C₆ alkyl-halogen; —NHC₁-C₆ alkyl-C₃-C₈ heterocycloalkyl; C₁-C₆ alkoxy; amino; di-C₁-C₆ alkylamino; and C₃-C₈ heterocycloalkyl unsubstituted or substituted with C₁-C₆ alkyl.

In one embodiment, R₃ is C₆-C₁₀ heterocycloalkyl having a heteroatom N, C₆-C₁₀ aryl or C₆-C₁₀ heteroaryl having a heteroatom N, and the heterocycloalkyl, aryl or heteroaryl is substituted or unsubstituted with one or more groups selected from the group consisting of hydroxy; halogen; C₁-C₆ alkyl; —C₁-C₆ alkyl-hydroxy; —C₃-C₈ heterocycloalkyl-C₁-C₆ alkyl-hydroxy; —NHC₁-C₆ alkyl-O—C₁-C₆ alkyl-hydroxy; —NHC₁-C₆ alkyl-O—C₁-C₆ alkyl-halogen; —NHC₁-C₆ alkyl-C₃-C₈ heterocycloalkyl; C₁-C₆ alkoxy; amino; di-C₁-C₆ alkylamino; and C₃-C₈ heterocycloalkyl unsubstituted or substituted with C₁-C₆ alkyl.

In one embodiment, R₃ is phenyl, naphthyl, pyridine, piperazine, or imidazole, and the phenyl, naphthyl, pyridine, piperazine, or imidazole is substituted or unsubstituted with one or more groups selected from the group consisting of hydroxy; halogen; C₁-C₆ alkyl; —C₁-C₆ alkyl-hydroxy; -piperazine-C₁-C₆ alkyl-hydroxy; —NHC₁-C₆ alkyl-O—C₁-C₆ alkyl-hydroxy; —NHC₁-C₆ alkyl-O—C₁-C₆ alkyl-halogen; —NHC₁-C₆ alkyl-morpholine; C₁-C₆ alkoxy; amino; di-C₁-C₆ alkylamino; pyrrolidine; piperidine; piperazine unsubstituted or substituted with C₁-C₆ alkyl; and morpholine.

In one embodiment, the compound of the present invention may be a compound selected from the group consisting of the compounds listed in the following Table 1 or a pharmaceutically acceptable salt thereof.

TABLE 1

| Compound | Chemical name | Structural formula |
|---|---|---|
| 1 (1aaa) | 5-(2,4-dihydroxybenzylidene)- 1-methyl-3-phenyl-2- selenoxoimidazolidin-4-one | 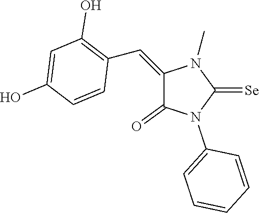 [SMILES: CN1C(=[Se])N(c2ccccc2)C(=O)/C1=C\c1ccc(O)cc1O] |
| 2 (1aab) | 5-(2,3-dihydroxybenzylidene)- 1-methyl-3-phenyl-2- selenoxoimidazolidin-4-one | 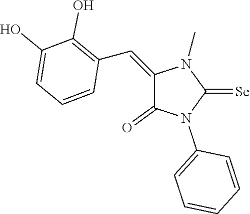 [SMILES: CN1C(=[Se])N(c2ccccc2)C(=O)/C1=C\c1cccc(O)c1O] |
| 3 (1aac) | 5-(4- (dimethylamino)benzylidene)-1- methyl-3-phenyl-2- selenoxoimidazolidin-4-one | 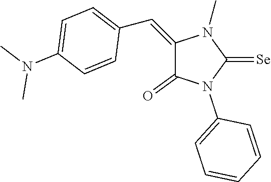 [SMILES: CN1C(=[Se])N(c2ccccc2)C(=O)/C1=C\c1ccc(N(C)C)cc1] |
| 4 (1aad) | 5-(3,4-dihydroxybenzylidene)- 1-methyl-3-phenyl-2- selenoxoimidazolidin-4-one | 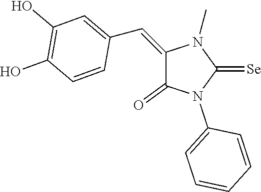 [SMILES: CN1C(=[Se])N(c2ccccc2)C(=O)/C1=C\c1ccc(O)c(O)c1] |
| 5 (1aae) | 5-(3,5-dihydroxybenzylidene)- 1-methyl-3-phenyl-2- selenoxoimidazolidin-4-one | 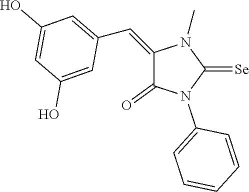 [SMILES: CN1C(=[Se])N(c2ccccc2)C(=O)/C1=C\c1cc(O)cc(O)c1] |
| 6 (1aaf) | 5-(2,6-dimethoxybenzylidene)- 1-methyl-3-phenyl-2- selenoxoimidazolidin-4-one | 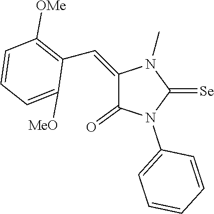 [SMILES: COc1cccc(OC)c1/C=C1\C(=O)N(c2ccccc2)C(=[Se])N1C] |
| 7 (1aak) | 1-methyl-3-phenyl-5-(4- pyrrolidin-1-yl)benzylidene-2- selenoxoimidazolidin-4-one | 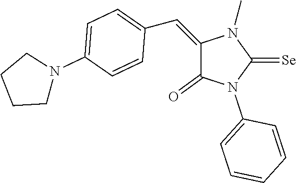 [SMILES: CN1C(=[Se])N(c2ccccc2)C(=O)/C1=C\c1ccc(N2CCCC2)cc1] |
| 8 (1aam) | 5-(3-hydroxybenzylidene)-1- methyl-3-phenyl-2- selenoxoimidazolidin-4-one | 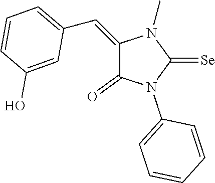 [SMILES: CN1C(=[Se])N(c2ccccc2)C(=O)/C1=C\c1cccc(O)c1] |
| 9 (1aan) | 1-methyl-3-phenyl-5-(pyridin- 3-ylmethylene)-2- selenoxoimidazolidin-4-one | 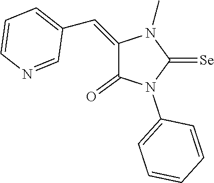 [SMILES: CN1C(=[Se])N(c2ccccc2)C(=O)/C1=C\c1cccnc1] |
| 10 (1aba) | 5-(2,4-dihydroxybenzylidene)- 1-methyl-3-(4-tolyl)-2- selenoxoimidazolidin-4-one | 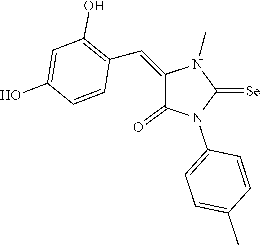 [SMILES: Cc1ccc(N2C(=O)/C(=C\c3ccc(O)cc3O)N(C)C2=[Se])cc1] |
| 11 (1abb) | 5-(2,3-dihydroxybenzylidene)- 1-methyl-3-(4-tolyl)-2- selenoxoimidazolidin-4-one | 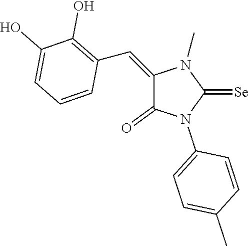 [SMILES: Cc1ccc(N2C(=O)/C(=C\c3cccc(O)c3O)N(C)C2=[Se])cc1] |
| 12 (1abc) | 5-(4- (dimethylamino)benzylidene)-1- methyl-3-(4-tolyl)-2- selenoxoimidazolidin-4-one | 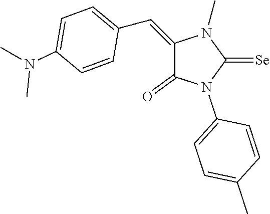 [SMILES: Cc1ccc(N2C(=O)/C(=C\c3ccc(N(C)C)cc3)N(C)C2=[Se])cc1] |
| 13 (1abd) | 5-(3,4-dihydroxybenzylidene)- 1-methyl-3-(4-tolyl)-2- selenoxoimidazolidin-4-one | 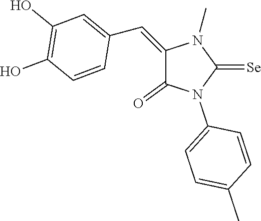 [SMILES: Cc1ccc(N2C(=O)/C(=C\c3ccc(O)c(O)c3)N(C)C2=[Se])cc1] |
| 14 (1abe) | 5-(3,5-dihydroxybenzylidene)- 1-methyl-3-(4-tolyl)-2- selenoxoimidazolidin-4-one | 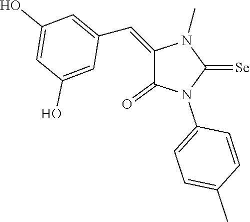 [SMILES: Cc1ccc(N2C(=O)/C(=C\c3cc(O)cc(O)c3)N(C)C2=[Se])cc1] |
| 15 (1abh) | 1-methyl-5-(4-piperidin-1- yl)benzylidene-3-(4-tolyl)-2- selenoxoimidazolidin-4-one | 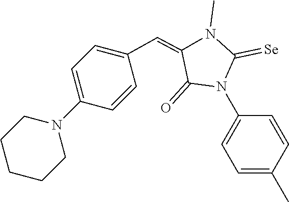 [SMILES: Cc1ccc(N2C(=O)/C(=C\c3ccc(N4CCCCC4)cc3)N(C)C2=[Se])cc1] |
| 16 (1abi) | 5-(4-bromobenzylidene)-1- methyl-2-selenoxo-3-(4- tolyl)imidazolidin-4-one | 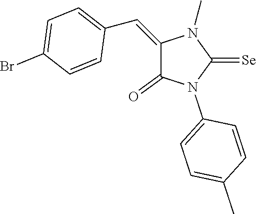 [SMILES: Cc1ccc(N2C(=O)/C(=C\c3ccc(Br)cc3)N(C)C2=[Se])cc1] |
| 17 (1abk) | 1-methyl-5-(4-pyrrolidin-1- yl)benzylidene-2-selenoxo-3- (4-tolyl)imidazolidin-4-one | 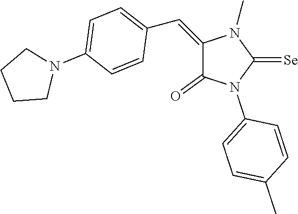 [SMILES: Cc1ccc(N2C(=O)/C(=C\c3ccc(N4CCCC4)cc3)N(C)C2=[Se])cc1] |
| 18 (1abl) | 5-(4-hydroxybenzylidene)-1- methyl-2-selenoxo-3-(4- tolyl)imidazolidin-4-one | 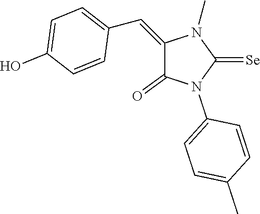 [SMILES: Cc1ccc(N2C(=O)/C(=C\c3ccc(O)cc3)N(C)C2=[Se])cc1] |
| 19 (1abn) | 1-methyl-5-(pyridin-3- ylmethylene)-2-selenoxo-3-(4- tolyl)imidazolidin-4-one | 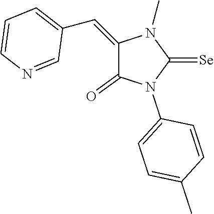 [SMILES: Cc1ccc(N2C(=O)/C(=C\c3cccnc3)N(C)C2=[Se])cc1] |
| 20 (1abt) | 1-methyl-5-(4-(4- methylpiperazin-1- yl)benzylidene)-2-selenoxo-3- (4-tolyl)imidazolin-4-one | 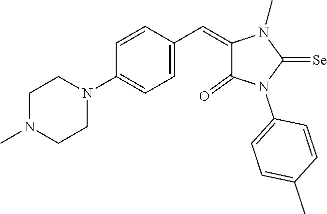 [SMILES: Cc1ccc(N2C(=O)/C(=C\c3ccc(N4CCN(C)CC4)cc3)N(C)C2=[Se])cc1] |
| 21 (1abu) | 5-(4-(4-(2- hydroxyethyl)piperazin-1- yl)benzylidene)-1-methyl-2- selenoxo-3-(4- tolyl)imidazolin-4-one | 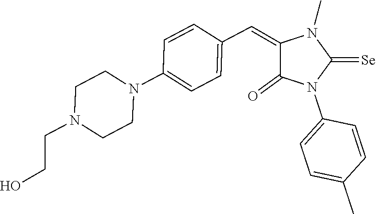 [SMILES: Cc1ccc(N2C(=O)/C(=C\c3ccc(N4CCN(CCO)CC4)cc3)N(C)C2=[Se])cc1] |
| 22 (1abv) | 5-(4-( (2-(2- hydroxyethoxy) ethyl)amino) benzylidene)-1-methyl-2-selenoxo- 3-(4-tolyl)imidazolin-4-one | 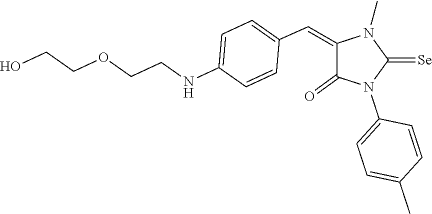 [SMILES: Cc1ccc(N2C(=O)/C(=C\c3ccc(NCCOCCO)cc3)N(C)C2=[Se])cc1] |
| 23 (1abw) | 1-methyl-5-(4-((2- morpholinoethyl)amino)benzylidene)- 2-selenoxo-3-(4- tolyl)imidazolin-4-one | 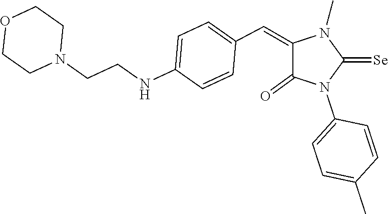 [SMILES: Cc1ccc(N2C(=O)/C(=C\c3ccc(NCCN4CCOCC4)cc3)N(C)C2=[Se])cc1] |
| 24 (1aca) | 5-(2,4-dihydroxybenzylidene)- 3-(4-ethylphenyl)-1-methyl-2- selenoxoimidazolidin-4-one | 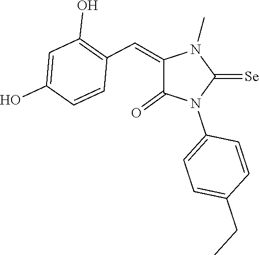 [SMILES: CCc1ccc(N2C(=O)/C(=C\c3ccc(O)cc3O)N(C)C2=[Se])cc1] |
| 25 (1acc) | 5-(4- (dimethylamino)benzylidene)-3- (4-ethylphenyl)-1-methyl-2- selenoxoimidazolidin-4-one | 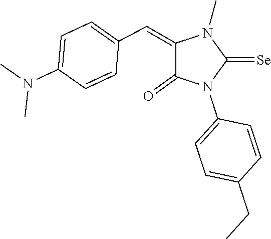 [SMILES: CCc1ccc(N2C(=O)/C(=C\c3ccc(N(C)C)cc3)N(C)C2=[Se])cc1] |
| 26 (1acd) | 5-(3,4-dihydroxybenzylidene)- 3-(4-ethylphenyl)-1-methyl-2- selenoxoimidazolidin-4-one | 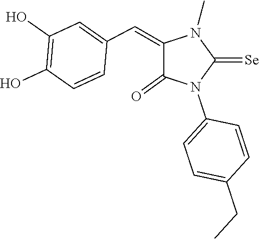 [SMILES: CCc1ccc(N2C(=O)/C(=C\c3ccc(O)c(O)c3)N(C)C2=[Se])cc1] |
| 27 (1ace) | 5-(3,5-dihydroxybenzylidene)- 3-(4-ethylphenyl)-1-methyl-2- selenoxoimidazolidin-4-one | 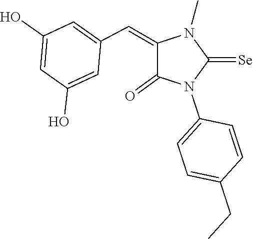 [SMILES: CCc1ccc(N2C(=O)/C(=C\c3cc(O)cc(O)c3)N(C)C2=[Se])cc1] |
| 28 (1ach) | 3-(4-ethylphenyl)-1-methyl-5- (4-(piperidin-1- yl)benzylidene)-2- selenoxoimidazolidin-4-one | 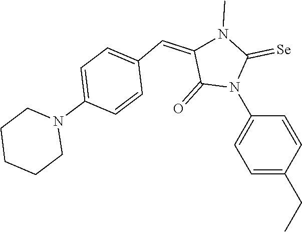 [SMILES: CCc1ccc(N2C(=O)/C(=C\c3ccc(N4CCCCC4)cc3)N(C)C2=[Se])cc1] |
| 29 (1aci) | 5-(4-bromobenzylidene)-3-(4- ethylphenyl)-1-methyl-2- selenoxoimidazolidin-4-one | 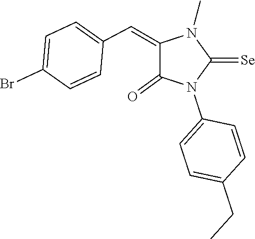 [SMILES: CCc1ccc(N2C(=O)/C(=C\c3ccc(Br)cc3)N(C)C2=[Se])cc1] |
| 30 (1ack) | 3-(4-ethylphenyl)-1-methyl-5- (4-pyrrolidin-1- yl)benzylidene)-2- selenoxoimidazolidin-4-one | 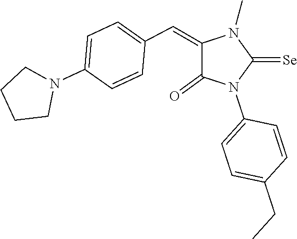 [SMILES: CCc1ccc(N2C(=O)/C(=C\c3ccc(N4CCCC4)cc3)N(C)C2=[Se])cc1] |
| 31 (1acn) | 3-(4-ethylphenyl)-1-methyl-5- (pyridin-3-ylmethylene)-2- selenoxoimidazolidin-4-one | 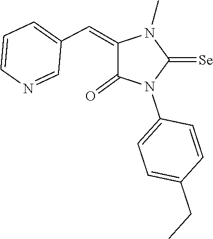 [SMILES: CCc1ccc(N2C(=O)/C(=C\c3cccnc3)N(C)C2=[Se])cc1] |
| 32 (1acs) | 3-(4-ethylphenyl)-1-methyl-5- (4-morpholidinobenzylidene)-2- selenoxoimidazolidin-4-one | 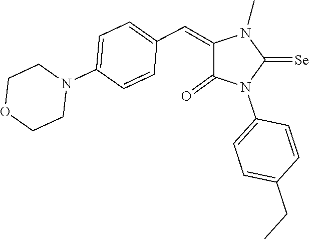 [SMILES: CCc1ccc(N2C(=O)/C(=C\c3ccc(N4CCOCC4)cc3)N(C)C2=[Se])cc1] |
| 33 (1acu) | 3-(4-ethylphenyl)-5-(4-(4-(2- hydroxyethyl)piperazin-1- yl)benzylidene)-1-methyl-2- selenoxoimidazolidin-4-one | 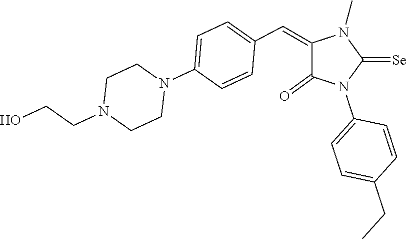 [SMILES: CCc1ccc(N2C(=O)/C(=C\c3ccc(N4CCN(CCO)CC4)cc3)N(C)C2=[Se])cc1] |
| 34 (1acy) | 3-(4-ethylphenyl)-5-(4-( (2-(2- fluoroethoxy) ethyl)amino) benzylidene)-1-methyl-2- selenoxoimidazolidin-4-one | 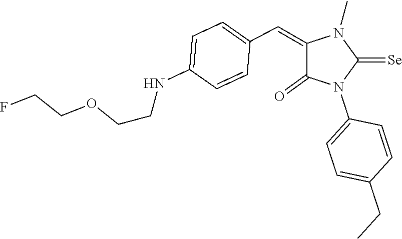 [SMILES: CCc1ccc(N2C(=O)/C(=C\c3ccc(NCCOCCF)cc3)N(C)C2=[Se])cc1] |
| 35 (1ada) | 3-(4-chlorophenyl)-5-(2,4- dihydroxybenzylidene)-1- methyl-2-selenoxoimidazolidin- 4-one | 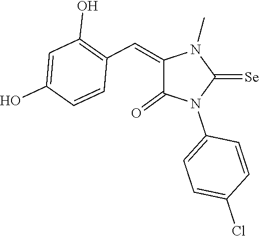 [SMILES: CN1C(=[Se])N(c2ccc(Cl)cc2)C(=O)/C1=C\c1ccc(O)cc1O] |
| 36 (1adb) | 3-(4-chlorophenyl)-5-(2,3- dihydroxybenzylidene)-1- methyl-2-selenoxoimidazolidin- 4-one | 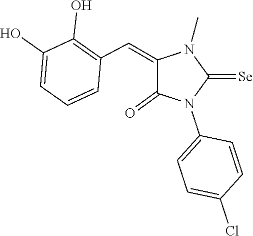 [SMILES: CN1C(=[Se])N(c2ccc(Cl)cc2)C(=O)/C1=C\c1cccc(O)c1O] |
| 37 (1add) | 3-(4-chlorophenyl)-5-(3,4- dihydroxybenzylidene)-1- methyl-2-selenoxoimidazolidin- 4-one | 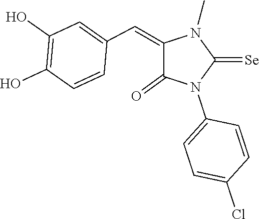 [SMILES: CN1C(=[Se])N(c2ccc(Cl)cc2)C(=O)/C1=C\c1ccc(O)c(O)c1] |
| 38 (1ade) | 3-(4-chlorophenyl)-5-(3,5- dihydroxybenzylidene)-1- methyl-2-selenoxoimidazolidin- 4-one | 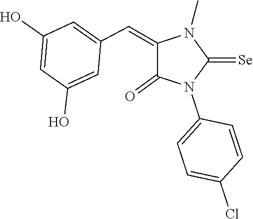 [SMILES: CN1C(=[Se])N(c2ccc(Cl)cc2)C(=O)/C1=C\c1cc(O)cc(O)c1] |
| 39 (1adn) | 3-(4-chlorophenyl)-1-methyl-5- (pyridin-3-ylmethylene)-2- selenoxoimidazolidin-4-one | 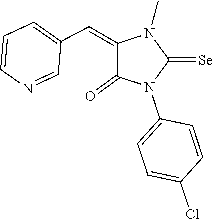 [SMILES: CN1C(=[Se])N(c2ccc(Cl)cc2)C(=O)/C1=C\c1cccnc1] |
| 40 (1aeb) | 3-(3-chlorophenyl)-5-(2,3- dihydroxybenzylidene)-1- methyl-2-selenoxoimidazolidin- 4-one | 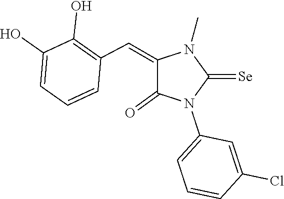 [SMILES: CN1C(=[Se])N(c2cccc(Cl)c2)C(=O)/C1=C\c1cccc(O)c1O] |
| 41 (1aha) | 5-(2,4-dihydroxybenzylidene)- 1-methyl-3-phenethyl-2- selenoxoimidazolidin-4-one | 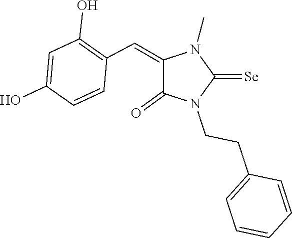 [SMILES: CN1C(=[Se])N(CCc2ccccc2)C(=O)/C1=C\c1ccc(O)cc1O] |
| 42 (1ahb) | 5-(2,3-dihydroxybenzylidene)- 1-methyl-3-phenethyl-2- selenoxoimidazolidin-4-one | 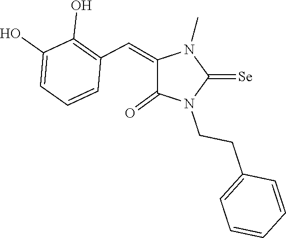 [SMILES: CN1C(=[Se])N(CCc2ccccc2)C(=O)/C1=C\c1cccc(O)c1O] |
| 43 (1ahc) | 5-(4- (dimethylamino)benzylidene)-1- methyl-3-phenethyl-2- selenoxoimidazolidin-4-one | 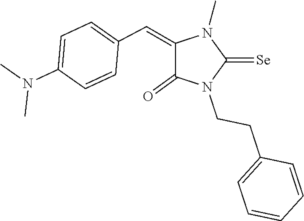 [SMILES: CN1C(=[Se])N(CCc2ccccc2)C(=O)/C1=C\c1ccc(N(C)C)cc1] |
| 44 (1ahe) | 5-(3,5-dihydroxybenzylidene)- 1-methyl-3-phenethyl-2- selenoxoimidazolidin-4-one | 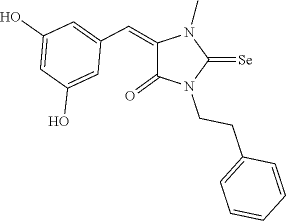 [SMILES: CN1C(=[Se])N(CCc2ccccc2)C(=O)/C1=C\c1cc(O)cc(O)c1] |
| 45 (1ahh) | 1-methyl-3-phenethyl-5-(4- (piperidin-1-yl)benzylidene)- 2-selenoxoimidazolidin-4-one | 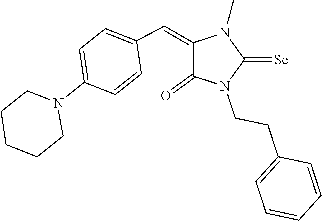 [SMILES: CN1C(=[Se])N(CCc2ccccc2)C(=O)/C1=C\c1ccc(N2CCCCC2)cc1] |
| 46 (1ahj) | 5-(4-chlorobenzylidene)-1- methyl-3-phenethyl-2- selenoxoimidazolidin-4-one | 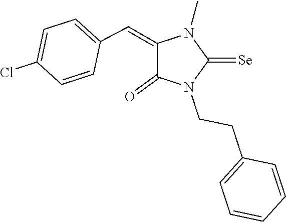 [SMILES: CN1C(=[Se])N(CCc2ccccc2)C(=O)/C1=C\c1ccc(Cl)cc1] |
| 47 (1ahk) | 1-methyl-3-phenethyl-5-(4- (pyrrolidin-1-yl)benzylidene)- 2-selenoxoimidazolidin-4-one | 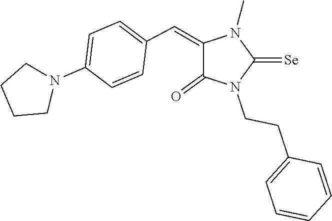 [SMILES: CN1C(=[Se])N(CCc2ccccc2)C(=O)/C1=C\c1ccc(N2CCCC2)cc1] |
| 48 (1ahl) | 5-(4-hydroxybenzylidene)-1- methyl-3-phenethyl-2- selenoxoimidazolidin-4-one | 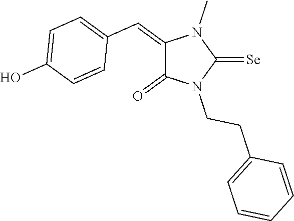 [SMILES: CN1C(=[Se])N(CCc2ccccc2)C(=O)/C1=C\c1ccc(O)cc1] |
| 49 (1ahm) | 5-(3-hydroxybenzylidene)-1- methyl-3-phenethyl-2- selenoxoimidazolidin-4-one | 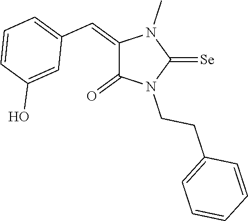 [SMILES: CN1C(=[Se])N(CCc2ccccc2)C(=O)/C1=C\c1cccc(O)c1] |
| 50 (1ahp) | 1-methyl-5-(naphthalen-2- ylmethylene)-3-phenethyl-2- selenoxoimidazolidin-4-one | 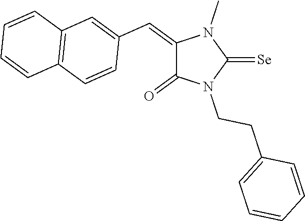 [SMILES: CN1C(=[Se])N(CCc2ccccc2)C(=O)/C1=C\c1ccc2ccccc2c1] |
| 51 (1ahq) | 1-methyl-5-( (1-methyl-1H- imidazol-5-yl)methylene)-3- phenethyl-2- selenoxoimidazolidin-4-one | 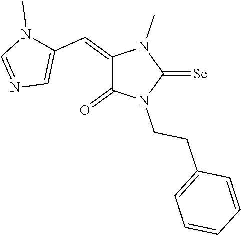 [SMILES: CN1C(=[Se])N(CCc2ccccc2)C(=O)/C1=C\c1cncn1C] |
| 52 (1ahs) | 1-methyl-5-(4- morpholinobenzylidene)-3- phenethyl-2- selenoxoimidazolidin-4-one | 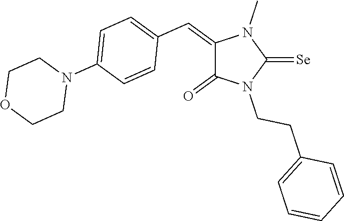 [SMILES: CN1C(=[Se])N(CCc2ccccc2)C(=O)/C1=C\c1ccc(N2CCOCC2)cc1] |
| 53 (1ahu) | 5-(4-(4-(2- hydroxyethyl)piperazin-1- yl)benzylidene)-1-methyl-3- phenethyl-2- selenoxoimidazolidin-4-one | 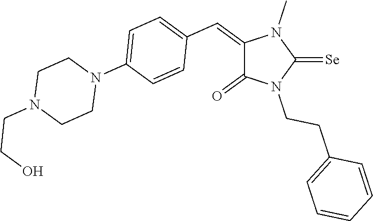 [SMILES: CN1C(=[Se])N(CCc2ccccc2)C(=O)/C1=C\c1ccc(N2CCN(CCO)CC2)cc1] |
| 54 (1ahw) | 1-methyl-5-(2- (morpholinoethyl)amino) benzylidene)-3-phenethyl-2- selenoxoimidazolidin-4-one | 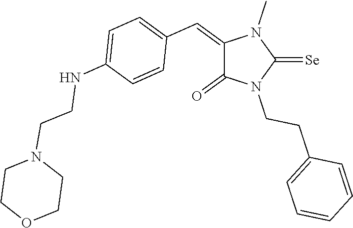 [SMILES: CN1C(=[Se])N(CCc2ccccc2)C(=O)/C1=C\c1ccc(NCCN2CCOCC2)cc1] |
| 55 (1aia) | 3-cyclohexy1-5-(2,4- dihydroxybenzylidene)-1- methyl-2-selenoxoimidazolidin- 4-one | 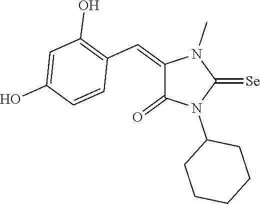 [SMILES: CN1C(=[Se])N(C2CCCCC2)C(=O)/C1=C\c1ccc(O)cc1O] |
| 56 (1aib) | 3-cyclohexyl-5-(2,3- dihydroxybenzylidene)-1- methyl-2-selenoxoimidazolidin- 4-one | 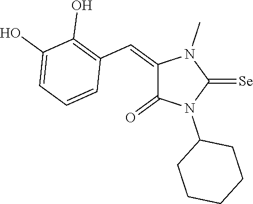 [SMILES: CN1C(=[Se])N(C2CCCCC2)C(=O)/C1=C\c1cccc(O)c1O] |
| 57 (1aic) | 3-cyclohexyl-5-(4- dimethylamino)benzylidene)-1- methyl-2-selenoxoimidazolidin- 4-one | 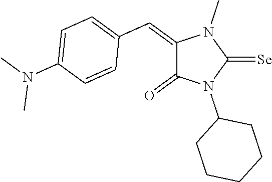 [SMILES: CN1C(=[Se])N(C2CCCCC2)C(=O)/C1=C\c1ccc(N(C)C)cc1] |
| 58 (1aie) | 3-cyclohexyl-5-(3,5- dihydroxybenzylidene)-1- methyl-2-selenoxoimidazolidin- 4-one | 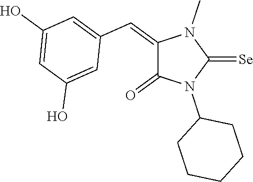 [SMILES: CN1C(=[Se])N(C2CCCCC2)C(=O)/C1=C\c1cc(O)cc(O)c1] |
| 59 (1aja) | 5-(2,4-dihydroxybenzylidene)- 3-hexyl-1-methyl-2- selenoxoimidazolidin-4-one | 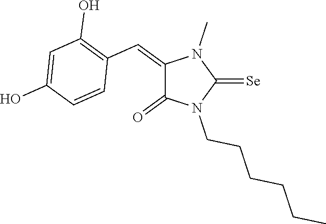 [SMILES: CCCCCCN1C(=O)/C(=C\c2ccc(O)cc2O)N(C)C1=[Se]] |
| 60 (1ajb) | 5-(2,3-dihydroxybenzylidene)- 3-hexyl-1-methyl-2- selenoxoimidazolidin-4-one | 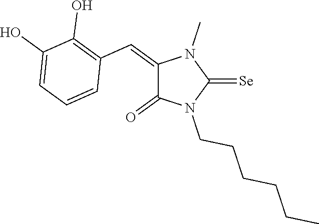 [SMILES: CCCCCCN1C(=O)/C(=C\c2cccc(O)c2O)N(C)C1=[Se]] |
| 61 (1ajc) | 5-(4- (dimethylamino)benzylidene)-3- hexyl-1-methyl-2- selenoxoimidazolidin-4-one | 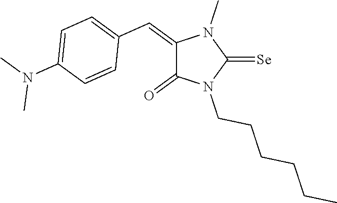 [SMILES: CCCCCCN1C(=O)/C(=C\c2ccc(N(C)C)cc2)N(C)C1=[Se]] |
| 62 (1aje) | 5-(3,5-dihydroxybenzylidene)- 3-hexyl-1-methyl-2- selenoxoimidazolidin-4-one | 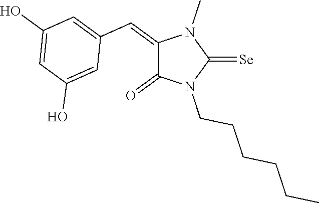 [SMILES: CCCCCCN1C(=O)/C(=C\c2cc(O)cc(O)c2)N(C)C1=[Se]] |
| 63 (1ajs) | 3-hexyl-1-methyl-5-(4- morpholinobenzylidene)-2- selenoxoimidazolidin-4-one | 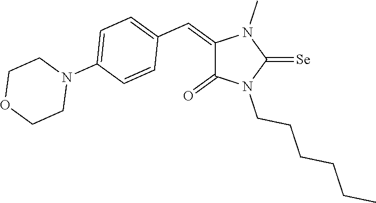 [SMILES: CCCCCCN1C(=O)/C(=C\c2ccc(N3CCOCC3)cc2)N(C)C1=[Se]] |
| 64 (1ajv) | 3-hexyl-5-(4-( (2-(2- hydroxyethoxy) ethyl)amino) benzylidene)-1-methyl-2- selenoxoimidazolidin-4-one | 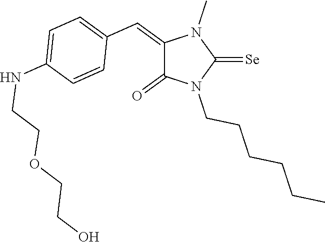 [SMILES: CCCCCCN1C(=O)/C(=C\c2ccc(NCCOCCO)cc2)N(C)C1=[Se]] |
| 65 (1ajw) | 3-hexyl-1-methyl-5-(4-((2- morpholinoethyl)amino) benzylidene)- 2-selenoxoimidazolidin-4-one | 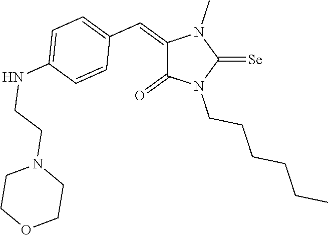 [SMILES: CCCCCCN1C(=O)/C(=C\c2ccc(NCCN3CCOCC3)cc2)N(C)C1=[Se]] |
| 66 (1ajx) | 5-(4-fluorobenzylidene)-3- hexyl-1-methyl-2- selenoxoimidazolidin-4-one | 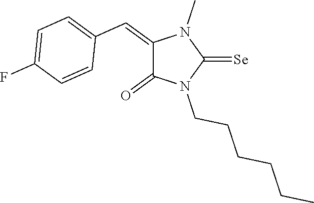 [SMILES: CCCCCCN1C(=O)/C(=C\c2ccc(F)cc2)N(C)C1=[Se]] |
| 67 (1ajy) | 5-(4-((2-(2- fluoroethoxy)ethyl)amino) benzylidene)-3-hexyl-1-methyl-2- selenoxoimidazolidin-4-one | 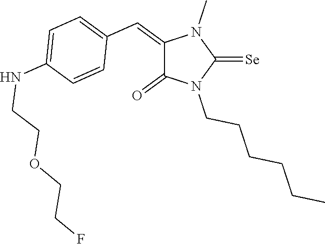 [SMILES: CCCCCCN1C(=O)/C(=C\c2ccc(NCCOCCF)cc2)N(C)C1=[Se]] |
| 68 (1aka) | 3-cyclopentyl-5-(2,4- dihydroxybenzylidene)-1- methyl-2-selenoxoimidazolidin- 4-one | 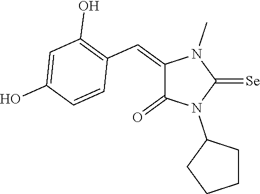 [SMILES: CN1C(=[Se])N(C2CCCC2)C(=O)/C1=C\c1ccc(O)cc1O] |
| 69 (1akb) | 3-cyclopentyl-5-(2,3- dihydroxybenzylidene)-1- methyl-2-selenoxoimidazolidin- 4-one | 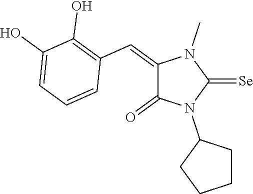 [SMILES: CN1C(=[Se])N(C2CCCC2)C(=O)/C1=C\c1cccc(O)c1O] |
| 70 (1akc) | 3-cyclopentyl-5-(4- (dimethylamino)benzylidene)-1- methyl-2-selenoxoimidazolidin- 4-one | 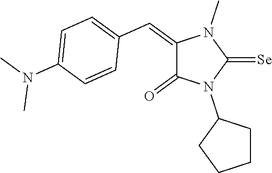 [SMILES: CN1C(=[Se])N(C2CCCC2)C(=O)/C1=C\c1ccc(N(C)C)cc1] |
| 71 (1akd) | 3-cyclopentyl-5-(3,4- dihydroxybenzylidene)-1- methyl-2-selenoxoimidazolidin- 4-one | 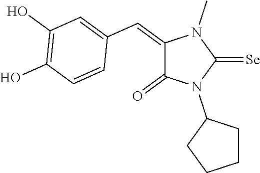 [SMILES: CN1C(=[Se])N(C2CCCC2)C(=O)/C1=C\c1ccc(O)c(O)c1] |
| 72 (1ake) | 3-cyclopentyl-5-(3,5- dihydroxybenzylidene)-1- methyl-2-selenoxoimidazolidin- 4-one | 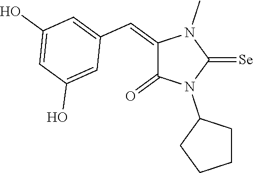 [SMILES: CN1C(=[Se])N(C2CCCC2)C(=O)/C1=C\c1cc(O)cc(O)c1] |
| 73 (1ala) | 5-(2,4-dihydroxybenzylidene)- 3-(4-fluorophenyl)-1-methyl-2- selenoxoimidazolidin-4-one | 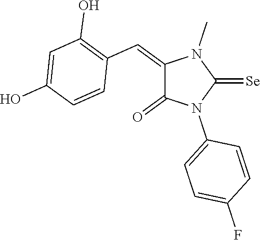 [SMILES: CN1C(=[Se])N(c2ccc(F)cc2)C(=O)/C1=C\c1ccc(O)cc1O] |
| 74 (1alb) | 5-(2,3-dihydroxybenzylidene)- 3-(4-fluorophenyl)-1-methyl-2- selenoxoimidazolidin-4-one | 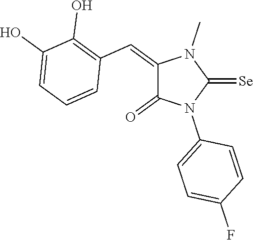 [SMILES: CN1C(=[Se])N(c2ccc(F)cc2)C(=O)/C1=C\c1cccc(O)c1O] |
| 75 (1alc) | 5-(4- (dimethylamino)benzylidene)-3- (4-fluorophenyl)-1-methyl-2- selenoxoimidazolidin-4-one | 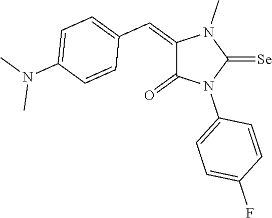 [SMILES: CN1C(=[Se])N(c2ccc(F)cc2)C(=O)/C1=C\c1ccc(N(C)C)cc1] |
| 76 (1ald) | 5-(3,4-dihydroxybenzylidene)- 3-(4-fluorophenyl)-1-methyl-2- selenoxoimidazolidin-4-one | 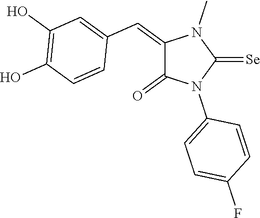 [SMILES: CN1C(=[Se])N(c2ccc(F)cc2)C(=O)/C1=C\c1ccc(O)c(O)c1] |
| 77 (1ale) | 5-(3,5-dihydroxybenzylidene)- 3-(4-fluorophenyl)-1-methyl-2- selenoxoimidazolidin-4-one | 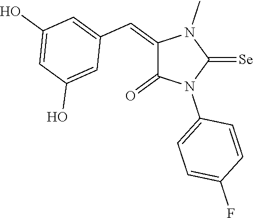 [SMILES: CN1C(=[Se])N(c2ccc(F)cc2)C(=O)/C1=C\c1cc(O)cc(O)c1] |
| 78 (1ama) | 5-(2,4-dihydroxybenzylidene)- 1-methyl-3-octyl-2- selenoxoimidazolidin-4-one | 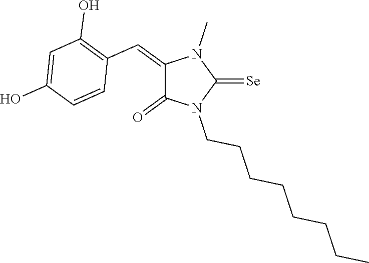 [SMILES: CCCCCCCCN1C(=O)/C(=C\c2ccc(O)cc2O)N(C)C1=[Se]] |
| 79 (1ame) | 5-(3,5-dihydroxybenzylidene)- 1-methyl-3-octyl-2- selenoxoimidazolidin-4-one | 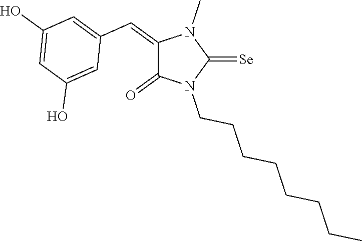 [SMILES: CCCCCCCCN1C(=O)/C(=C\c2cc(O)cc(O)c2)N(C)C1=[Se]] |
| 80 (lana) | 3-decyl-5-(2,4- dihydroxybenzylidene)-1- methyl-2-selenoxoimidazolidin- 4-one | 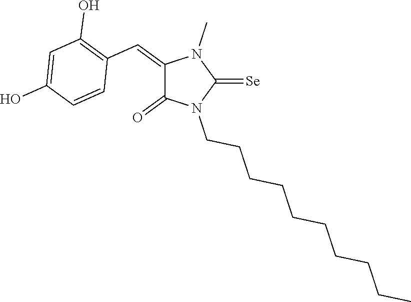 [SMILES: CCCCCCCCCCN1C(=O)/C(=C\c2ccc(O)cc2O)N(C)C1=[Se]] |
| 81 (1apa) | 5-(2,4-dihydroxybenzylidene)- 1-methyl-3-(2- morpholinoethyl)-2- selenoxoimidazolidin-4-one | 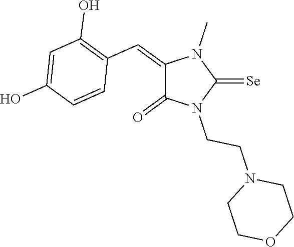 [SMILES: CN1C(=[Se])N(CCN2CCOCC2)C(=O)/C1=C\c1ccc(O)cc1O] |
| 82 (1ape) | 5-(3,5-dihydroxybenzylidene)- 1-methyl-3-(2- morpholinoethyl)-2- selenoxoimidazolidin-4-one | 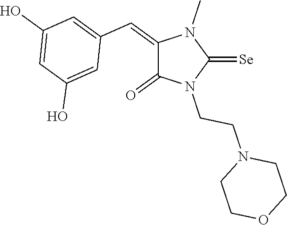 [SMILES: CN1C(=[Se])N(CCN2CCOCC2)C(=O)/C1=C\c1cc(O)cc(O)c1] |
| 83 (1aqa) | 5-(2,4-dihydroxybenzylidene)- 1-methyl-3-(3- morpholinopropyl)-2- selenoxoimidazolidin-4-one | 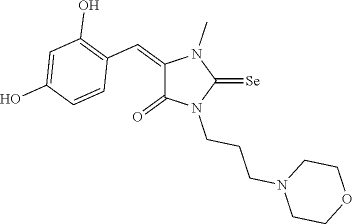 [SMILES: CN1C(=[Se])N(CCCN2CCOCC2)C(=O)/C1=C\c1ccc(O)cc1O] |
| 84 (1aqe) | 5-(3,5-dihydroxybenzylidene)- 1-methyl-3-(3- morpholinopropyl)-2- selenoxoimidazolidin-4-one | 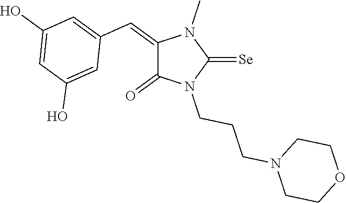 [SMILES: CN1C(=[Se])N(CCCN2CCOCC2)C(=O)/C1=C\c1cc(O)cc(O)c1] |
| 85 (1aqj) | 5-(4-chlorobenzylidene)-1- methyl-3-(3-morpholinopropyl)- 2-selenoxoimidazolidin-4-one | 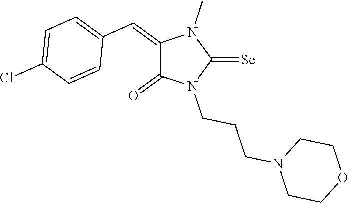 [SMILES: CN1C(=[Se])N(CCCN2CCOCC2)C(=O)/C1=C\c1ccc(Cl)cc1] |
| 86 (1aqx) | 5-(4-fluorobenzylidene)-1- methyl-3-(3-morpholinopropyl)- 2-selenoxoimidazolidin-4-one | 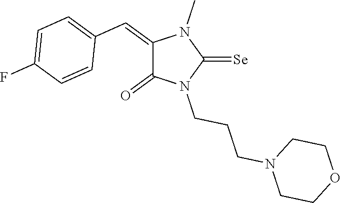 [SMILES: CN1C(=[Se])N(CCCN2CCOCC2)C(=O)/C1=C\c1ccc(F)cc1] |
| 87 (lara) | 5-(2,4-dihydroxybenzylidene)- 3-(5-fluorohexyl)-1-methyl-2- selenoxoimidazolidin-4-one | 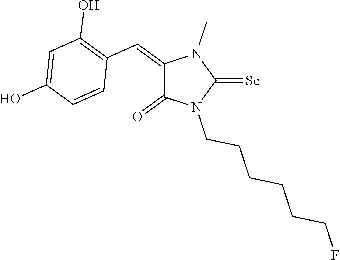 [SMILES: CN1C(=[Se])N(CCCCCCF)C(=O)/C1=C\c1ccc(O)cc1O] |
| 88 (lare) | 5-(3,5-dihydroxybenzylidene)- 3-(5-fluorohexyl)-1-methyl-2- selenoxoimidazolidin-4-one | 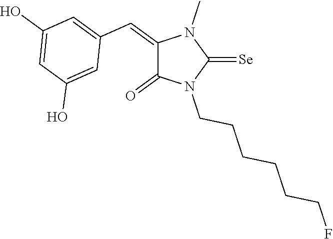 [SMILES: CN1C(=[Se])N(CCCCCCF)C(=O)/C1=C\c1cc(O)cc(O)c1] |
| 89 (lasa) | 5-(2,4-dihydroxybenzylidene)- 3-(5-fluoropentyl)-1-methyl-2- selenoxoimidazolidin-4-one | 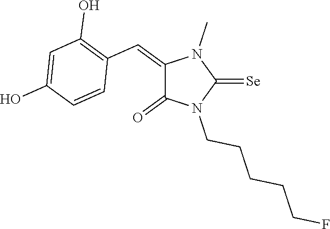 [SMILES: CN1C(=[Se])N(CCCCCF)C(=O)/C1=C\c1ccc(O)cc1O] |
| 90 (lase) | 5-(3,5-dihydroxybenzylidene)- 3-(5-fluoropentyl)-1-methyl-2- selenoxoimidazolidin-4-one | 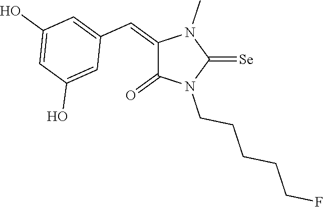 [SMILES: CN1C(=[Se])N(CCCCCF)C(=O)/C1=C\c1cc(O)cc(O)c1] |
| 91 (lata) | 3-butyl-5-(2,4- dihydroxybenzylidene)-1- methyl-2-selenoxoimidazolidin- 4-one | 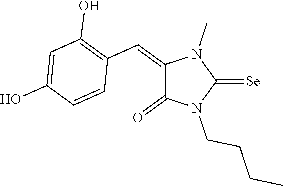 [SMILES: CCCCN1C(=O)/C(=C\c2ccc(O)cc2O)N(C)C1=[Se]] |
| 92 (late) | 3-butyl-5-(3,5- dihydroxybenzylidene)-1- methyl-2-selenoxoimidazolidin- 4-one | 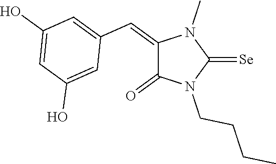 [SMILES: CCCCN1C(=O)/C(=C\c2cc(O)cc(O)c2)N(C)C1=[Se]] |
| 93 (1aua) | 5-(2,4-dihydroxybenzylidene)- 1-methyl-3-penty1-2- selenoxoimidazolidin-4-one | 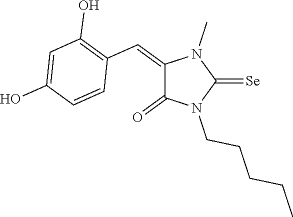 [SMILES: CCCCCN1C(=O)/C(=C\c2ccc(O)cc2O)N(C)C1=[Se]] |
| 94 (1aue) | 5-(3,5-dihydroxybenzylidene)- 1-methyl-3-penty1-2- selenoxoimidazolidin-4-one | 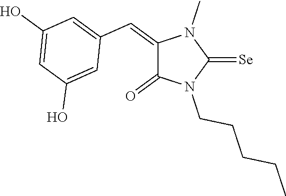 [SMILES: CCCCCN1C(=O)/C(=C\c2cc(O)cc(O)c2)N(C)C1=[Se]] |
| 95 (1baa) | 5-(2,4-dihydroxybenzylidene)- 1-ethyl-3-phenyl-2- selenoxoimidazolidin-4-one | 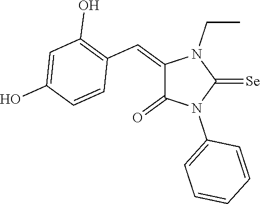 [SMILES: CCN1C(=[Se])N(c2ccccc2)C(=O)/C1=C\c1ccc(O)cc1O] |
| 96 (1bab) | 5-(2,3-dihydroxybenzylidene)- 1-ethyl-3-phenyl-2- selenoxoimidazolidin-4-one | 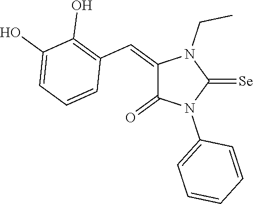 [SMILES: CCN1C(=[Se])N(c2ccccc2)C(=O)/C1=C\c1cccc(O)c1O] |
| 97 (1bac) | 5-(4- (dimethylamino)benzylidene)-1- ethyl-3-phenyl-2- selenoxoimidazolidin-4-one | 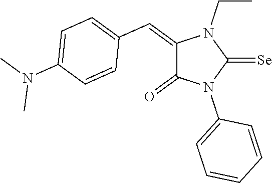 [SMILES: CCN1C(=[Se])N(c2ccccc2)C(=O)/C1=C\c1ccc(N(C)C)cc1] |
| 98 (1bad) | 5-(3,4-dihydroxybenzylidene)- 1-ethyl-3-phenyl-2- selenoxoimidazolidin-4-one | 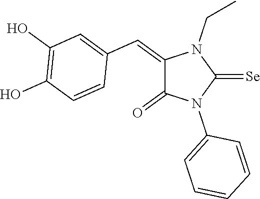 [SMILES: CCN1C(=[Se])N(c2ccccc2)C(=O)/C1=C\c1ccc(O)c(O)c1] |
| 99 (1bae) | 5-(3,5-dihydroxybenzylidene)- 1-ethyl-3-phenyl-2- selenoxoimidazolidin-4-one | 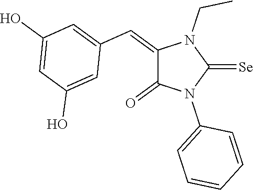 [SMILES: CCN1C(=[Se])N(c2ccccc2)C(=O)/C1=C\c1cc(O)cc(O)c1] |
| 100 (1bce) | 5-(3,5-dihydroxybenzylidene)- 1-ethyl-3-(4-ethylphenyl)-2- selenoxoimidazolidin-4-one | 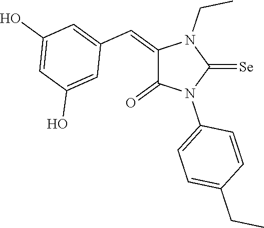 [SMILES: CCc1ccc(N2C(=O)/C(=C\c3cc(O)cc(O)c3)N(CC)C2=[Se])cc1] |
| 101 (1bci) | 5-(4-bromobenzylidene)-1- ethyl-3-(4-ethylphenyl)-2- selenoxoimidazolidin-4-one | 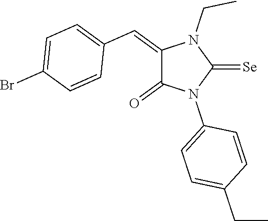 [SMILES: CCc1ccc(N2C(=O)/C(=C\c3ccc(Br)cc3)N(CC)C2=[Se])cc1] |
| 102 (1bck) | 1-ethyl-3-(4-ethylphenyl)-5- (4-pyrrolidin-1- yl)benzylidene-2- selenoxoimidazolidin-4-one | 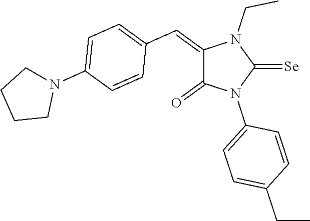 [SMILES: CCc1ccc(N2C(=O)/C(=C\c3ccc(N4CCCC4)cc3)N(CC)C2=[Se])cc1] |
| 103 (1caa) | 1-benzyl-5-(2,4- dihydroxybenzylidene)-3- phenyl-2-selenoxoimidazolidin- 4-one | 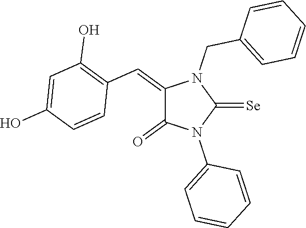 [SMILES: O=C1/C(=C\c2ccc(O)cc2O)N(Cc2ccccc2)C(=[Se])N1c1ccccc1] |
| 104 (1cab) | 1-benzyl-5-(2,3- dihydroxybenzylidene)-3- phenyl-2-selenoxoimidazolidin- 4-one | 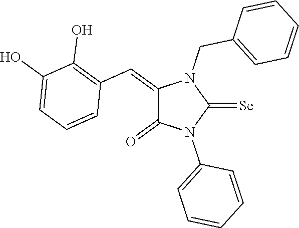 [SMILES: O=C1/C(=C\c2cccc(O)c2O)N(Cc2ccccc2)C(=[Se])N1c1ccccc1] |
| 105 (1cac) | 1-benzyl-5-(4- (dimethylamino)benzylidene)-3- phenyl-2-selenoxoimidazolidin- 4-one | 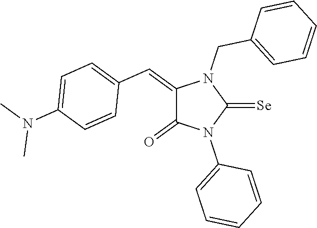 [SMILES: CN(C)c1ccc(/C=C2\C(=O)N(c3ccccc3)C(=[Se])N2Cc2ccccc2)cc1] |
| 106 (1cad) | 1-benzyl-5-(3,4- dihydroxybenzylidene)-3- phenyl-2-selenoxoimidazolidin- 4-one | 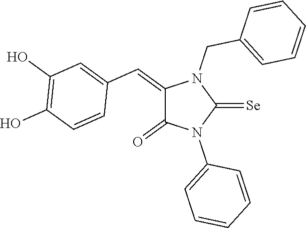 [SMILES: O=C1/C(=C\c2ccc(O)c(O)c2)N(Cc2ccccc2)C(=[Se])N1c1ccccc1] |
| 107 (1cae) | 1-benzyl-5-(3,5- dihydroxybenzylidene)-3- phenyl-2-selenoxoimidazolidin- 4-one | 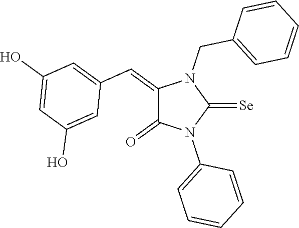 [SMILES: O=C1/C(=C\c2cc(O)cc(O)c2)N(Cc2ccccc2)C(=[Se])N1c1ccccc1] |
| 108 (1cca) | 1-benzyl-5-(2,4- dihydroxybenzylidene)-3-(4- ethylphenyl)-2- selenoxoimidazolidin-4-one | 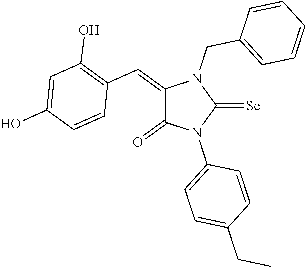 [SMILES: CCc1ccc(N2C(=O)/C(=C\c3ccc(O)cc3O)N(Cc3ccccc3)C2=[Se])cc1] |
| 109 (1ccd) | 1-benzyl-5-(3,4- dihydroxybenzylidene)-3-(4- ethylphenyl)-2- selenoxoimidazolidin-4-one | 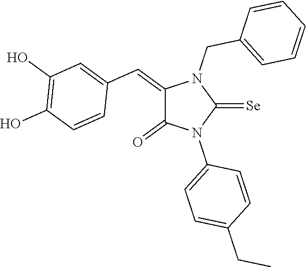 [SMILES: CCc1ccc(N2C(=O)/C(=C\c3ccc(O)c(O)c3)N(Cc3ccccc3)C2=[Se])cc1] |
| 110 (1cce) | 1-benzyl-5-(3,5- dihydroxybenzylidene)-3-(4- ethylphenyl)-2- selenoxoimidazolidin-4-one | 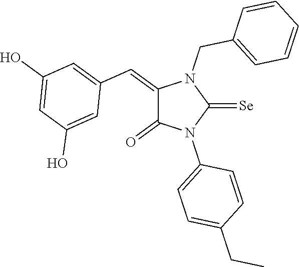 [SMILES: CCc1ccc(N2C(=O)/C(=C\c3cc(O)cc(O)c3)N(Cc3ccccc3)C2=[Se])cc1] |
| 111 (1cdb) | 1-benzyl-3-(4-chlorophenyl)-5- (2,3-dihydroxybenzylidene)-2- selenoxoimidazolidin-4-one | 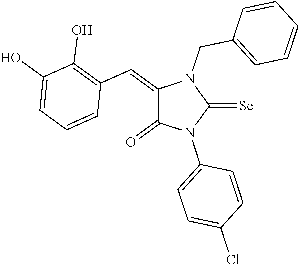 [SMILES: O=C1/C(=C\c2cccc(O)c2O)N(Cc2ccccc2)C(=[Se])N1c1ccc(Cl)cc1] |
| 112 (1cdd) | 1-benzyl-3-(4-chlorophenyl)-5- (3,4-dihydroxybenzylidene)-2- selenoxoimidazolidin-4-one | 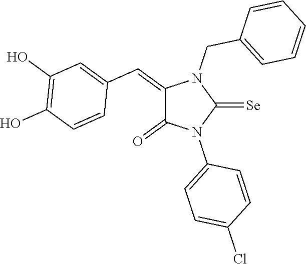 [SMILES: O=C1/C(=C\c2ccc(O)c(O)c2)N(Cc2ccccc2)C(=[Se])N1c1ccc(Cl)cc1] |
| 113 (1aak HCl) | 1-methyl-3-phenyl-5-(4- (pyrrolidin-1-yl)benzylidene)- 2-selenoxoimidazolidin-4-one • hydrochloride | 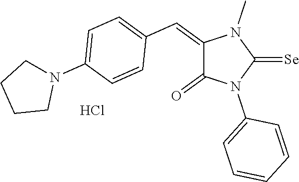 [SMILES: CN1C(=[Se])N(c2ccccc2)C(=O)/C1=C\c1ccc(N2CCCC2)cc1 ; Cl] |
| 114 (1abk HCl) | 1-methyl-5-(4-(pyrrolidin-1- yl)benzylidene)-2-selenoxo-3- (4-tolyl)imidazolidin-4-one • hydrochloride | 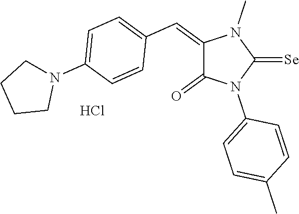 [SMILES: Cc1ccc(N2C(=O)/C(=C\c3ccc(N4CCCC4)cc3)N(C)C2=[Se])cc1 ; Cl] |
| 115 (1abt HCl) | 1-methyl-5-(4-(4- methylpiperazin-1- yl)benzylidene)-2-selenoxo-3- (4-tolyl)-imidazolidin-4-one • hydrochloride | 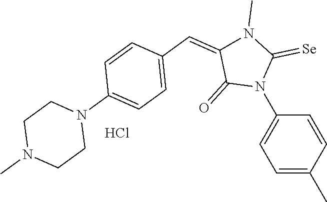 |
| 116 (1abw HCl) | 1-methyl-5-(4-((2- morpholinoethyl)amino)benzylidene)- 2-selenoxo-3-(4- tolyl)imidazolin-4-one • hydrochloride | 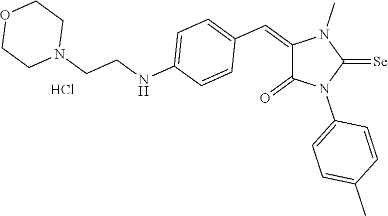 [SMILES: Cc1ccc(N2C(=O)/C(=C\c3ccc(NCCN4CCOCC4)cc3)N(C)C2=[Se])cc1 ; Cl] |
| 117 (1ach HCl) | 3-(4-ethylphenyl)-1-methyl-5- (4-(piperidin-1- yl)benzylidene)-2- selenoxoimidazolidin-4-one • hydrochloride | 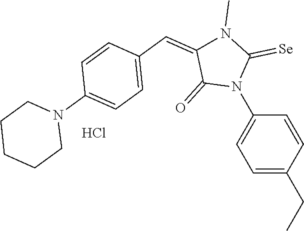 [SMILES: CCc1ccc(N2C(=O)/C(=C\c3ccc(N4CCCCC4)cc3)N(C)C2=[Se])cc1 ; Cl] |
| 118 (lajw HCl) | 3-hexyl-1-methyl-5-(4-( (2- morpholinoethyl)amino)benzylidene)- 2-selenoxoimidazolidin-4-one • hydrochloride | 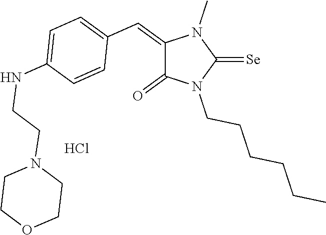 [SMILES: CCCCCCN1C(=O)/C(=C\c2ccc(NCCN3CCOCC3)cc2)N(C)C1=[Se] ; Cl] |
| 119 (1ahk HCl) | 1-methyl-3-phenethyl-5-(4- (pyrrolidin-1-yl)benzylidene)- 2-selenoxoimidazolidin-4-one • hydrochloride | 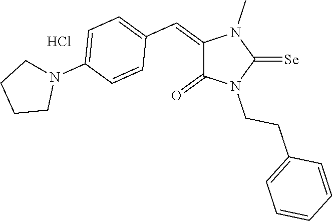 [SMILES: CN1C(=[Se])N(CCc2ccccc2)C(=O)/C1=C\c1ccc(N2CCCC2)cc1 ; Cl] |
| 120 (1akc HCl) | 3-cyclopentyl-5-(4- (dimethylamino)benzylidene)-1- methyl-2-selenoxoimidazolidin- 4-one • hydrochloride | 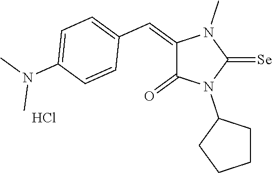 [SMILES: CN1C(=[Se])N(C2CCCC2)C(=O)/C1=C\c1ccc(N(C)C)cc1 ; Cl] |
| 121 (1aqa HCl) | 5-(2,4-dihydroxybenzylidene)- 1-methyl-3-(3- morpholinopropyl)-2- selenoxoimidazolidin-4-one • hydrochloride | 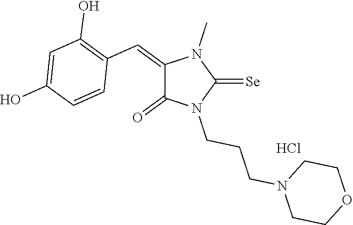 [SMILES: CN1C(=[Se])N(CCCN2CCOCC2)C(=O)/C1=C\c1ccc(O)cc1O ; Cl] |
| 122 (1aqx HCl) | 5-(4-fluorobenzylidene)-1- methyl-3-(3-morpholinopropyl)- 2-selenoxoimidazolidin-4-one • hydrochloride | 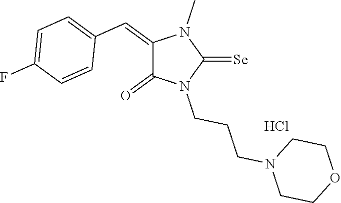 [SMILES: CN1C(=[Se])N(CCCN2CCOCC2)C(=O)/C1=C\c1ccc(F)cc1 ; Cl] |

In one embodiment, the inflammatory diseases may be selected from the group consisting of sepsis, septic shock, inflammatory bowel disease (IBD), peritonitis, nephritis, diabetic nephropathy, diabetic retinopathy, acute bronchitis, chronic bronchitis, osteoarthritis, ankylosing spondylitis, chronic obstructive pulmonary disease (COPD), rheumatoid arthritis, acute lung injury, and bronchopulmonary dysplasia.

Furthermore, the inflammatory bowel disease may be ulcerative colitis (UC) or Crohn's disease.

Furthermore, the present invention provides a method for preparing a compound of Chemical Formula 1 or a pharmaceutically acceptable salt thereof, comprising:
reacting an amine compound of Chemical Formula 5 with formic acid to produce a formamide compound of Chemical Formula 6;
reacting the formamide compound with selenium (Se) to produce an isoselenocyanate compound of Formula 7 under conditions comprising one or more selected from triphosgene, dichloromethane, and triethylamine;
reacting the isoselenocyanate compound in a solvent with an amino acid compound of Chemical Formula 8 to produce a 1-alkyl-2-selenoxoimidazolidin-4-one compound of Chemical Formula 9; and
reacting the 1-alkyl-2-selenoxoimidazolidin-4-one compound with an aldehyde compound of Chemical Formula 10 to produce the compound of Formula 1.

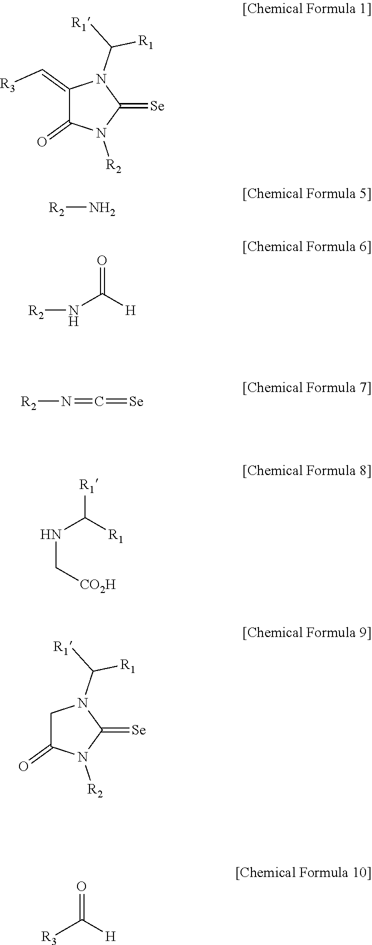

In the above formula,
R₁ and R₁′ are each independently selected from the group consisting of hydrogen, alkyl, cycloalkyl, heterocycloalkyl, aryl, heteroaryl, benzyl, and a phenethyl, or R₁ and R₁′ may combine together to form a ring;
R₂ and R₃ are each independently halo, cyano, alkyl, alkoxy, alkenyl, alkynyl, cycloalkyl, heterocycloalkyl, heterocycloalkyl-alkyl, aryl, aryl-alkyl, or heteroaryl;
and the alkyl, alkoxy, alkenyl, alkynyl, cycloalkyl, heterocycloalkyl, heterocycloalkyl-alkyl, aryl, aryl-alkyl, or heteroaryl is optionally substituted or unsubstituted with one or more groups selected from the group consisting of hydroxy; halogen; alkyl; -alkyl-hydroxy; -heterocycloalkyl-alkyl-hydroxy; —NH alkyl-O-alkyl-hydroxy; —NH alkyl-O-alkyl-halogen; —NH alkyl-heterocycloalkyl; alkoxy; amino; dialkylamino; nitro; cyano; carbonyl; cycloalkyl; heterocycloalkyl unsubstituted or substituted with alkyl; aryl; and heteroaryl.

In one embodiment, the above

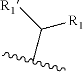

may be

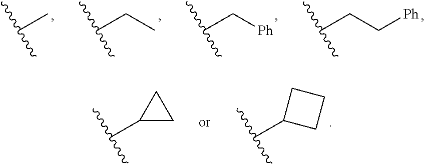

In one embodiment, R₂ may be C₁-C₁₂ alkyl; C₁-C₆ alkyl substituted with halogen; C₃-C₈ cycloalkyl; —C₁-C₆ alkyl-morpholine; phenyl unsubstituted or substituted with halogen or C₁-C₆ alkyl; or —C₁-C₆ alkyl-phenyl.

In one embodiment, R₃ is phenyl, naphthyl, pyridine, piperazine, or imidazole, and the phenyl, naphthyl, pyridine, piperazine, or imidazole is substituted or unsubstituted with one or more groups selected from the group consisting of hydroxy; halogen; C₁-C₆ alkyl; —C₁-C₆ alkyl-hydroxy; -piperazine-C₁-C₆ alkyl-hydroxy; —NHC₁-C₆ alkyl-O—C₁-C₆ alkyl-hydroxy; —NHC₁-C₆ alkyl-O—C₁-C₆ alkyl-halogen; —NHC₁-C₆ alkyl-morpholine; C₁-C₆ alkoxy; amino; di-C₁-C₆ alkylamino; pyrrolidine; piperidine; piperazine unsubstituted or substituted with C₁-C₆ alkyl; and morpholine.

In one embodiment, the solvent may be one or more selected from the group consisting of dioxane, methanol, ethanol, acetonitrile, tetrahydrofuran (THF), dimethylformamide (DMF), dimethyl sulfoxide (DMSO), and dichloroethylene (DCE).

### Advantageous Effects of Invention

The novel compound of the present invention, 1-alkyl-5-arylidene-2-selenoxoimidazolidin-4-one, or its derivatives, can be effectively utilized for the prevention, amelioration, or treatment of inflammatory diseases by inhibiting the NOX enzyme that generates reactive oxygen species and induces inflammation, and by modulating Nrf2.

Furthermore, the present invention provides a composition for the prevention, amelioration, or treatment of inflammatory diseases, comprising 1-alkyl-5-arylidene-2-selenoxoimidazolidin-4-one and its derivatives, or a pharmaceutically acceptable salt thereof.

### BRIEF DESCRIPTION OF DRAWINGS

FIG. 1 illustrates a schematic reaction formula depicting the method of synthesizing 1-alkyl-5-arylidene-2-selenoxoimidazolidin-4-one and its derivatives.

FIG. 2 illustrates the effect of compound 1aaa (compound 1) of the present invention on inhibiting the generation of reactive oxygen species (ROS) induced by MPP+ treatment.

FIG. 3 illustrates the effect of compound 1aaa (compound 1) of the present invention on inhibiting the generation of reactive oxygen species (ROS) induced by rotenone treatment.

FIG. 4 illustrates the cell viability induced by compounds 1aaa (compound 1), 1aca (compound 24), 1ace (compound 27), 1ahe (compound 44), and 1aje (compound 62) of the present invention in a case of being treated with rotenone.

FIG. 5 illustrates the cell viability induced by compounds 1aaa (compound 1), 1aca (compound 24), 1ace (compound 27), 1ahe (compound 44), and 1aje (compound 62) of the present invention in a case of being treated with MPP+.

FIG. 6 illustrates the amount of nuclear Nrf-2 as determined by Western blot analysis.

FIG. 7 illustrates the activation of BV2 cell lines induced by LPS treatment and the inhibitory effect of compound 1aaa (compound 1) of the present invention on such activation.

FIG. 8 illustrates the expression level of IL-1β as determined by Western blot analysis.

FIG. 9 illustrates the expression level of COX2 as determined by Western blot analysis.

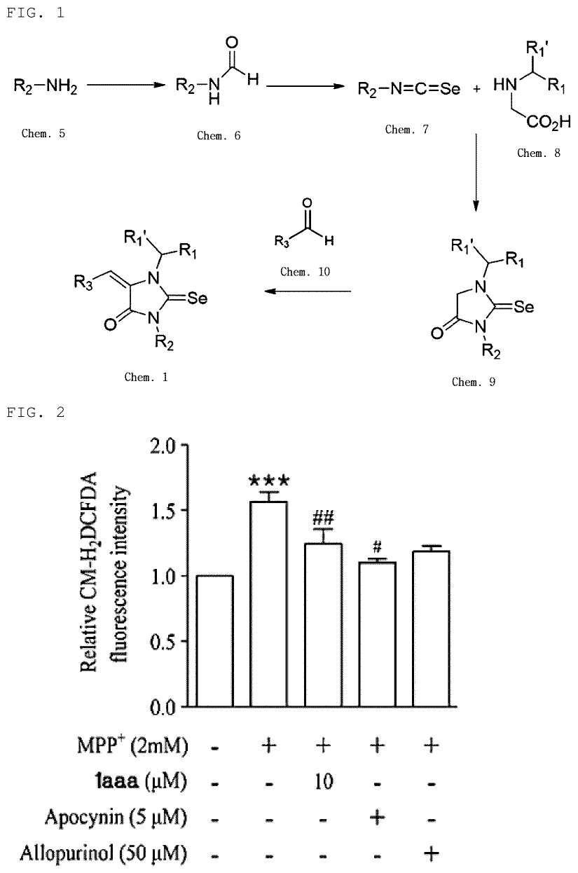

### BEST MODE FOR CARRYING OUT THE INVENTION

Hereinafter, embodiments and examples of the present disclosure will be described in detail with reference to the accompanying drawings so that those skilled in the art can easily practice the present invention. However, the present disclosure may be implemented in various forms and is not limited to the embodiments and examples described herein.

Throughout the specification of the present disclosure, when any part is described as “comprising” a certain component, it means that other components can be included unless specifically stated otherwise, rather than excluding other components.

In cases where temporal relationships are described, for example, using terms such as “after,” “following,” “subsequent to,” “before” or the like, these descriptions can include non-continuous events unless the terms “immediately” or “directly” are explicitly used to denote continuous sequences.

The terms “first,” “second,” and the like are used to describe various components, but these components are not limited by these terms. These terms are only used to distinguish one component from another. Therefore, a first component described hereinafter could be a second component within the scope of the present invention.

The term “at least one” should be understood to include all possible combinations of one or more of the related items. For example, “at least one of a first item, a second item, and a third item” means not only each of the first item, the second item, and the third item individually, but also any combination of two or more of the first item, the second item, and the third item.

The features of various embodiments of the present invention can be combined or integrated with each other partially or entirely, and can be technically interconnected and operated in various ways. Each embodiment may be practiced independently of the other embodiments or in conjunction with them.

As used herein, the term “alkyl” refers to a hydrocarbon having primary, secondary, tertiary, and/or quaternary carbon atoms, and includes saturated aliphatic groups that can be straight-chain, branched, cyclic, or a combination thereof. For example, an alkyl group may have 1 to 20 carbon atoms (i.e., C₁-C₂₀ alkyl), 1 to 10 carbon atoms (i.e., C₁-C₁₀ alkyl), or 1 to 6 carbon atoms (i.e., C₁-C₆ alkyl). Unless otherwise defined, in a preferred embodiment, an alkyl refers to a C₁-C₆ alkyl. Suitable examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃) CH₂CH₃), 2-methyl-2-propyl (t-Bu, t-butyl, —C(CH₃) 3), 1-pentyl (n-pentyl, —CH₂CH₂CH₂CH₂CH₃), 2-pentyl (—CH(CH₃) CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃) CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃) CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃) CH(CH₃) CH₂CH₃), 4-methyl-2-pentyl (—CH(CH₃) CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), and octyl (—(CH₂)₇CH₃).

Furthermore, the term “alkyl” as used throughout the specification, embodiments, and claims is intended to include both unsubstituted and substituted alkyl groups, the latter of which refers to alkyl residues having one or more substituents that replace hydrogen atoms on the carbon backbone, such as haloalkyl groups including trifluoromethyl and 2,2,2-trifluoroethyl.

As used herein, the term “cycloalkyl” refers to a substituted or unsubstituted, non-aromatic saturated or unsaturated monocyclic, bicyclic, or polycyclic ring where each atom in the ring is carbon. Cycloalkyl can be polycyclic cycloalkyl comprising two or more rings in which one or more carbons are common to adjacent rings. Polycyclic cycloalkyl can be a fused ring system, spirocyclic ring system, or bridged ring system, where one or more rings are cycloalkyl, and other rings can be, for example, cycloalkyl, aryl, heteroaryl, and/or heterocycloalkyl as defined herein. Suitable examples of cycloalkyl include, but are not limited to, cyclopropyl, cyclobutyl, cyclopentyl, cyclohexyl, cycloheptyl, and cyclooctyl.

As used herein, the term “heterocycloalkyl” refers to a substituted or unsubstituted, non-aromatic saturated or partially saturated monocyclic, bicyclic, or polycyclic ring containing one or more heteroatoms within the ring. Heterocycloalkyl can be polycyclic heterocycloalkyl comprising two or more rings in which one or more atoms are common to adjacent rings. Polycyclic heterocycloalkyl can be a fused ring system, spirocyclic ring system, or bridged ring system, where one or more rings are heterocycloalkyl, and other rings can be, for example, cycloalkyl, aryl, heteroaryl, and/or heterocycloalkyl as defined herein. Suitable examples of heterocycloalkyl include, but are not limited to, piperidinyl, piperazinyl, pyrrolidinyl, morpholinyl, lactonyl, lactamyl, azetidinyl, dihydropyridinyl, dihydroindolyl, tetrahydropyridinyl (piperidinyl), tetrahydrothiophenyl, sulfur-oxidized tetrahydrothiophenyl, indolinyl, 4-piperidinyl, 2-pyrrolidonyl, tetrahydrofuranyl, tetrahydroquinolinyl, tetrahydroisoquinolinyl, decahydroquinolinyl, octahydroisoquinolinyl, 6H-1,2,5-thiadiazinyl, 2H,6H-1,5,2-dithiazinyl, pyranyl, chromenyl, xanthenyl, phenoxathiinyl, 2H-pyrrolyl, 3H-indolyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, phenanthrolinyl, phenazinyl, phenothiazinyl, purazinyl, phenoxazinyl, isochromanyl, chromanyl, imidazolidinyl, pyrazolidinyl, pyrazolinyl, quinuclidinyl, and oxazolidinyl.

As used herein, the term “aryl” includes substituted or unsubstituted, monocyclic, bicyclic, or polycyclic monovalent or divalent aromatic hydrocarbon groups, where each atom in the ring is carbon. Preferably, the aryl ring is a 6- to 20-membered ring, a 6- to 14-membered ring, a 6- to 10-membered ring, or more preferably, a 6-membered ring. An aryl group may be a polycyclic ring system having two or more cyclic rings where two or more carbons are common to two adjacent rings, wherein one or more rings are aromatic, and, for example, other cyclic rings may be cycloalkyl, cycloalkenyl, cycloalkynyl, aryl, heteroaryl, and/or heterocycloalkyl. Examples of aryl groups include benzene, naphthalene, phenanthrene, anthracene, indene, indane, phenol, and aniline.

As used herein, the term “heteroaryl” refers to a substituted or unsubstituted, monocyclic, bicyclic, or polycyclic monovalent or divalent aromatic group containing one or more heteroatoms within the ring. Non-limiting examples of suitable heteroatoms that can be contained in the aromatic ring include oxygen, sulfur, and nitrogen. “Heteroaryl” may be a bicyclic or polycyclic ring system having two or more cyclic rings where two or more carbons are common to two adjacent rings, wherein one or more rings are heteroaromatic, and other cyclic rings may be, for example, cycloalkyl, cycloalkenyl, cycloalkynyl, aryl, heteroaryl, and/or heterocyclyl. Examples of “heteroaryl” include, but are not limited to, benzofuran, benzothiophene, pyrrole, furan, thiophene, imidazole, indole, isoindole, isoxazole, isothiazole, oxazole, thiazole, quinoline, isoquinoline, pyrazole, pyridine, pyrazine, pyridazine, and pyrimidine, each of which may be substituted or unsubstituted.

As used herein, the term “heteroarylalkyl” refers to an alkyl in which one or more hydrogen atoms are substituted with a heteroaryl group, also referred to as a heteroarylalkyl group.

As used herein, the term “alkoxy” can be represented by the chemical formula —O-alkyl, where the alkyl group is attached to the parent compound through an oxygen atom, and the alkyl group is as defined herein and may be substituted or unsubstituted. The alkyl group of the alkoxy may have, for example, 1 to 20 carbon atoms (i.e., C₁-C₂₀ alkoxy), 1 to 12 carbon atoms (i.e., C₁-C₁₂ alkoxy), 1 to 10 carbon atoms (i.e., C₁-C₁₀ alkoxy), or 1 to 6 carbon atoms (i.e., C₁-C₆ alkoxy). Suitable examples of alkoxy groups include methoxy (—O—CH₃ or —OMe), ethoxy (—OCH₂CH₃ or -OEt), and t-butoxy (—OC(CH₃)₃ or —O-tBu), but are not limited thereto.

As used herein, the terms “halo” and “halogen” both mean halogen and include chloro, fluoro, bromo, and iodo.

Unless specifically stated otherwise, the alkyl, cycloalkyl, heterocycloalkyl, aryl, heteroaryl, arylalkyl, heteroarylalkyl, and alkoxy groups include those in which one or more hydrogens are substituted with chemically substitutable substituents, such as halogen, alkyl, alkoxy, hydroxy (OH), thiol (SH), amino (NH2), and nitro (NO₂).

In the present invention, the term “derivative” refers to a compound that is obtained by chemically modifying a part of the original compound, where a specific substituent or hydrogen (H) is replaced by another atom or group of atoms.

In the present invention, the term “pharmaceutically acceptable salt” means a salt that is pharmaceutically acceptable and retains the desired pharmacological activity of the parent compound. The salt is not particularly limited as long as it is pharmaceutically acceptable.

The inflammatory diseases of the present invention may include one or more selected from the group consisting of sepsis, septic shock, inflammatory bowel disease (IBD), peritonitis, nephritis, diabetic nephropathy, diabetic retinopathy, acute bronchitis, chronic bronchitis, osteoarthritis, ankylosing spondylitis, chronic obstructive pulmonary disease (COPD), rheumatoid arthritis, acute lung injury, and bronchopulmonary dysplasia, but are not limited thereto. Additionally, the inflammatory bowel disease may be ulcerative colitis (UC) or Crohn's disease, but is not limited thereto.

The compounds of Chemical Formula 1 of the present invention, i.e., 1-alkyl-5-arylidene-2-selenoxoimidazolidin-4-one and its derivatives, or pharmaceutically acceptable salts thereof, can inhibit the generation of reactive oxygen species by reducing NADPH oxidase (NOX) in vivo, thereby preventing the induction of inflammation and eliminating the fundamental cause of inflammatory diseases.

The pharmaceutical composition of the present invention can be formulated and used in any form suitable for pharmaceutical preparations, including, but not limited to, oral dosage forms such as powders, granules, tablets, soft or hard capsules, suspensions, emulsions, syrups, and aerosols; topical preparations such as ointments and creams; suppositories; injections; and sterile injectable solutions, each according to conventional methods.

For the formulation, commonly used excipients such as fillers, extenders, binders, wetting agents, disintegrants, surfactants, and diluents may be additionally included. Examples of excipients that may be included in the pharmaceutical composition of the present invention include, but are not limited to, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, methyl hydroxybenzoate, propyl hydroxybenzoate, talc, magnesium stearate, and mineral oils. In addition to simple excipients, lubricants such as magnesium stearate and talc can also be used.

The pharmaceutical composition of the present invention can be administered orally or parenterally. When administered parenterally, it can be administered via intravenous injection, subcutaneous injection, intramuscular injection, intraperitoneal injection, or transdermal delivery.

As used in the present invention, the term “improvement” refers to any act that at least reduces the parameters related to the treated condition, such as the severity of symptoms.

When using the composition of the present invention as a food composition, the compound of Chemical Formula 1, i.e., 1-alkyl-5-arylidene-2-selenoxoimidazolidin-4-one and its derivatives, can be added as is or used together with other foods or food ingredients, and can be appropriately used according to conventional methods. The composition may include food supplement additives that are acceptable in food science, in addition to the active ingredient. The amount of the active ingredient mixed can be appropriately determined according to the intended use (prevention, health, or therapeutic treatment).

In the present invention, the term “food supplement additive” refers to components that can be added to food adjunctively and are added to manufacture health functional foods of each formulation, which can be appropriately selected and used by those skilled in the art. Examples of food supplement additives include various nutrients, vitamins, minerals (electrolytes), synthetic flavoring agents and natural flavoring agents, colorants and fillers, pectic acid and its salts, alginic acid and its salts, organic acids, protective colloidal thickeners, pH adjusters, stabilizers, preservatives, glycerin, alcohol, and carbonating agents used in carbonated beverages, but are not limited to these examples.

The food composition of the present invention may include health functional foods. The term “health functional food” as used in the present invention refers to food that is manufactured and processed into forms such as tablets, capsules, powders, granules, liquids, and pills using ingredients or components with beneficial functionality for the human body. Here, “functionality” refers to obtaining beneficial effects for health purposes such as regulating nutrients or exerting physiological actions on the structure and function of the human body. The health functional food of the present invention can be manufactured by methods commonly used in the art, and in the course of manufacturing, ingredients and components commonly added in the art can be used. Furthermore, the formulations of the health functional foods can be manufactured without limitation, as long as they are recognized as formulations of health functional foods. The food composition of the present invention can be manufactured in various forms of formulations, and unlike general medicines, it has the advantage of not causing side effects that may occur with long-term use of medicines since it uses food as a raw material. It is also highly portable, allowing the health functional foods of the present invention to be consumed as supplements to enhance the effects of treating inflammatory diseases.

Furthermore, there is no limitation on the types of health foods in which the composition of the present invention can be used. Additionally, the composition comprising as an active ingredient the compound of Chemical Formula 1 of the present invention, namely 1-alkyl-5-arylidene-2-selenoxazolidin-4-one and its derivatives, can be manufactured by mixing with appropriate auxiliary components and known additives that can be included in health functional foods according to the choice of those skilled in the art. Examples of foods to which the composition can be added include meat, sausages, bread, chocolate, candies, snacks, cookies, pizza, ramen, other noodles, gum, dairy products including ice cream, various soups, beverages, teas, drinks, alcoholic beverages, and vitamin complexes. Furthermore, the compound of Chemical Formula 1 of the present invention, i.e., 1-alkyl-5-arylidene-2-selenoxazolidin-4-one and its derivatives, can be added to and manufactured into extracts, teas, jellies, and juices, etc., which are prepared with the compound as the main ingredient.

Hereinafter, the present invention will be described in more detail through the following examples, but the following examples are for illustrative purposes only and are not intended to limit the scope of the present invention.

### EXAMPLES

### Example 1

### Preparation of 5-(2,4-dihydroxybenzylidene)-1-methyl-3-phenyl-2-selenoxoimidazolidin-4-one (1aaa, Compound 1)

### 1) Synthesis of N-phenylformamide

Aniline (20 g, 215 mmol) was added to formic acid (30 g, 644 mmol), and then the reaction was conducted under reflux at 100° C. for 4 hours and terminated. After ethyl acetate was added to the reaction mixture for dilution, the organic layer was washed with water, 0.1 N aqueous hydrochloric acid solution, saturated sodium hydrogen carbonate solution, and brine. The organic layer was dried over anhydrous sodium sulfate, filtered, and concentrated under reduced pressure to obtain the target compound (25 g, 96%).

¹H-NMR (300 MHz, CDCl₃) δ 9.06 (br s, 0.4H), 8.68 (d, J=11.4 Hz, 0.6H), 8.33 (s, 0.6H), 8.16 (br s, 0.4H), 7.42-7.62 (m, 1H), 7.00-7.41 (m, 4H).

MS (EI): m/z=121 [M+H]⁺.

### 2) Synthesis of phenyl isoselenocyanate

After N-phenylformamide (4 g, 33 mmol) was dissolved in methylene chloride (145 mL), triethylamine (20 mL, 4.3 mmol) and 4A MS (660 mg) were added. After triphosgene (5.3 g, 18 mmol) was dissolved in methylene chloride (25 mL), the solution was slowly added dropwise over 1 hour. The reaction mixture was refluxed for 4 h, then selenium (5.2 g, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (3.6 g, 60%).

¹H-NMR (300 MHz, CDCl₃) δ 7.42 (s, 5H).

MS (EI): m/z=183 [M+H]⁺.

### 3) Synthesis of 1-methyl-3-phenyl-2-selenoxoimidazolidin-4-one

Dioxane (60 mL) and sarcosine (120 mg, 1.4 mmol) were added to the compound (250 mg, 1.4 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (292 mg, 85%).

¹H-NMR (300 MHz, CDCl₃) δ 7.45-7.60 (m, 3H), 7.27-7.40 (m, 2H), 4.08 (s, 2H), 3.51 (s, 3H).

MS (EI): m/z=254 [M+H]⁺.

### 4) Synthesis of 5-(2,4-dihydroxybenzylidene)-1-methyl-3-phenyl-2-selenoxoimidazolidin-4-one (1aaa)

Dioxane (0.6 mL) was added to the compound (51 mg, 0.2 mmol) obtained in step 3) and 2,4-dihydroxybenzaldehyde (30 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (41 mg, 55%).

¹H-NMR (300 MHz, DMSO-d₆) δ 9.97 (s, 1H), 9.73 (s, 1H), 8.19 (d, J=8.7 Hz, 1H), 7.30-7.60 (m, 5H), 6.67 (s, 1H), 6.35 (d, J=2.2 Hz, 1H), 6.22 (dd, J=8.7, 2.2 Hz, 1H), 3.19 (s, 3H).

HRMS (ESI): m/z=375.0245 [M+H]⁺.

### Example 2

### Preparation of 5-(2,3-dihydroxybenzylidene)-1-methyl-3-phenyl-2-selenoxoimidazolidin-4-one (1aab, Compound 2)

Dioxane (0.6 mL) was added to the compound (51 mg, 0.2 mmol) obtained in step 3) of Example 1 and 2,3-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (34 mg, 48%).

¹H-NMR (300 MHz, CDCl₃) δ 7.44-7.60 (m, 3H), 7.32-7.40 (m, 2H), 7.02-7.15 (m, 2H), 6.87-6.96 (m, 1H), 6.75-6.86 (m, 1H), 5.30-6.50 (br s, 2H), 3.88 (s, 1H), 3.53 (s, 2H).

MS (ESI): m/z=397.0070 [M+Na]⁺.

### Example 3

### Preparation of 5-(4-(dimethylamino)benzylidene)-1-methyl-3-phenyl-2-selenoxoimidazolidin-4-one (1aac, Compound 3)

Dioxane (0.6 mL) was added to the compound (51 mg, 0.2 mmol) obtained in step 3) of Example 1 and 4-(dimethylamino)benzaldehyde (30 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (70 mg, 91%).

¹H-NMR (300 MHz, CDCl₃) δ 8.20 (d, J=9.1 Hz, 2H), 7.44-7.58 (m, 4H), 7.32-7.42 (m, 3H), 7.14 (s, 0.3H), 6.79 (s, 1H), 6.72 (d, J=8.9 Hz, 0.6H), 6.66 (d, J=9.1 Hz, 2H), 3.85 (s, 3H), 3.68 (s, 1H), 3.06 (s, 2H), 3.05 (s, 6H).

MS (ESI): m/z=386.0769 [M+H]⁺.

### Example 4

### Preparation of 5-(3,4-dihydroxybenzylidene)-1-methyl-3-phenyl-2-selenoxoimidazolidin-4-one (1aad, Compound 4)

Dioxane (0.6 mL) was added to the compound (51 mg, 0.2 mmol) obtained in step 3) of Example 1 and 3,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (34 mg, 45%).

¹H-NMR (300 MHz, DMSO-d₆) δ 9.80 (br s, 1H), 0.9.25 (br s, 1H), 7.97 (d, J=1.9 Hz, 1H), 7.42-7.65 (m, 4H), 7.30-7.40 (m, 2H), 7.10 (s, 1H), 6.78 (d, J=8.3 Hz, 1H), 3.77 (s, 3H).

MS (ESI): m/z=375.0245 [M+H]⁺.

### Example 5

### Preparation of 5-(3,5-dihydroxybenzylidene)-1-methyl-3-phenyl-2-selenoxoimidazolidin-4-one (1aae, Compound 5)

Dioxane (0.6 mL) was added to the compound (51 mg, 0.2 mmol) obtained in step 3) of Example 1 and 2,6-dimethoxybenzaldehyde (30 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (59 mg, 79%).

¹H-NMR (300 MHz, DMSO-d₆+CDCl₃) δ δ 9.12 (s, 1H), 8.99 (s, 1H), 7.30-7.65 (m, 5H), 6.70-7.20 (m, 2H), 6.30-6.55 (m, 2H), 3.83 (s, 1.3H), 3.59 (s, 1.7H).

MS (ESI): m/z=375.0246 [M+H]⁺.

### Example 6

### Preparation of 5-(2,6-dimethoxybenzylidene)-1-methyl-3-phenyl-2-selenoxoimidazolidin-4-one (1aaf, Compound 6)

Dioxane (0.6 mL) was added to the compound (51 mg, 0.2 mmol) obtained in step 3) of Example 1 and 2,6-dimethoxybenzaldehyde (37 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (79 mg, 89%).

¹H-NMR (300 MHz, CDCl₃) δ 7.27-7.58 (m, 5H), 6.99 (s, 1H), 6.77 (s, 0.6H), 6.61 (d, J=8.4 Hz, 2H), 6.55 (d, J=8.4 Hz, 1H), 3.87 (s, 4H), 3.84 (s, 1H), 3.82 (s, 2H), 3.45 (s, 2H).

MS (ESI): m/z=425.0376 [M+Na]⁺.

### Example 7

### Preparation of 1-methyl-3-phenyl-5-(4-pyrrolidin-1-yl)benzylidene-2-selenoxoimidazolidin-4-one (1aak, Compound 7)

Dioxane (0.6 mL) was added to the compound (51 mg, 0.2 mmol) obtained in step 3) of Example 1 and 4-(pyrrolidin-1-yl)benzaldehyde (39 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (61 mg, 74%).

¹H-NMR (300 MHz, DMSO-d6) δ 8.25 (d, J=8.9 Hz, 1H), 7.42-7.59 (m, 4H), 7.29-7.41 (m, 2H), 7.15 (s, 0.6H), 7.01 (s, 0.4H), 6.50-6.67 (m, 2H), 3.78 (s, 1.8H), 3.60 (s, 1.2H), 3.34 (br s, 4H), 1.90-2.05 (m, 4H).

MS (EI): m/z=411.0847 [M]⁺.

### Example 8

### Preparation of 5-(3-hydroxybenzylidene)-1-methyl-3-phenyl-2-selenoxoimidazolidin-4-one (1aam, Compound 8)

Dioxane (0.6 mL) was added to the compound (51 mg, 0.2 mmol) obtained in step 3) of Example 1 and 3-hydroxybenzaldehyde (28 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (55 mg, 77%).

¹H-NMR (300 MHz, DMSO-d⁶) δ 9.74 (br s, 0.55H), 9.59 (br s, 0.45H), 7.67 (br s, 0.6H), 7.33-7.58 (m, 5.4H), 7.17-7.31 (m, 1H), 7.16 (s, 0.45H), 7.02 (s, 0.55H), 6.80-7.00 (m, 2H), 3.77 (s, 1.4H), 3.44 (s, 1.6H).

MS (ESI): m/z=381.0114 [M+Na]⁺.

### Example 9

### Preparation of 1-methyl-3-phenyl-5-(pyridin-3-ylmethylene)-2-selenoxoimidazolidin-4-one (1aan, Compound 9)

Dioxane (0.6 mL) was added to the compound (51 mg, 0.2 mmol) obtained in step 3) of Example 1 and 3-pyridinecarboxaldehyde (24 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (44 mg, 64%).

¹H-NMR (300 MHz, CDCl₃) δ 8.50-9.10 (m, 4H), 7.20-7.90 (m, 5H), 7.09 (s, 0.4H), 6.78 (s, 0.6H), 3.86 (s, 1.8H), 3.53 (s, 1.2H).

MS (ESI): m/z=344.0304 [M+H]⁺.

### Example 10

### Preparation of 5-(2,4-dihydroxybenzylidene)-1-methyl-3-(4-tolyl)-2-selenoxoimidazolidin-4-one (1aba, Compound 10)

### 1) Synthesis of N-(4-tolyl)formamide

After 4-tolylamine (32 g, 215 mmol) was added to formic acid (30 g, 644 mmol), the reaction was conducted under reflux at 100° C. for 4 hours and terminated. After ethyl acetate was added to the reaction mixture for dilution, the organic layer was washed with water, 0.1 N aqueous hydrochloric acid solution, saturated sodium hydrogen carbonate solution, and brine. The organic layer was dried over anhydrous sodium sulfate, filtered, and concentrated under reduced pressure to obtain the target compound (28 g, 95%).

¹H-NMR (300 MHz, CDCl₃) δ 8.63 (d, J=10.0 Hz, 1H), 8.32 (s, 0.5H), 7.84 (br s, 0.5H), 7.42 (d, J=8.4 Hz, 1H), 7.08-7.23 (m, 2H), 6.99 (d, J=8.3 Hz, 1H), 2.33 (s, 1.5H), 2.31 (s, 1.5H).

MS (EI): m/z=135 [M]⁺.

### 2) Synthesis of 4-tolyl isoselenocyanate

After N-(4-tolyl)formamide (4.50 g, 33 mmol) was dissolved in methylene chloride (145 mL), triethylamine (20 mL, 4.30 mmol) and 4A MS (660 mg) were added. After triphosgene (5.3 g, 18 mmol) was dissolved in methylene chloride (25 mL), the solution was slowly added dropwise over 1 hour. The reaction mixture was refluxed for 4 h, then selenium (5.2 g, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (5.7 g, 88%).

¹H-NMR (300 MHz, CDCl₃) δ 7.11-7.22 (m, 5H), 2.36 (s, 3H).

MS (EI): m/z=260 [M+H]⁺.

### 3) Synthesis of 1-methyl-3-(4-tolyl)-2-selenoxoimidazolidin-4-on

Dioxane (170 mL) and sarcosine (346 mg, 3.89 mmol) were added to the compound (762 mg, 3.89 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (748 mg, 72%).

¹H-NMR (300 MHz, CDCl₃) δ 7.31 (d, J=8.4 Hz, 2H), 7.19 (d, J=8.3 Hz, 2H), 4.06 (s, 2H), 3.50 (s, 3H), 2.41 (s, 3H).

MS (EI): m/z=268 [M+H]⁺.

### 4) Synthesis of 5-(2,4-dihydroxybenzylidene)-1-methyl-3-(4-tolyl)-2-selenoxoimidazolidin-4-one (1aba)

Dioxane (0.6 mL) was added to the compound (53 mg, 0.2 mmol) obtained in step 3) and 2,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (47 mg, 61%).

¹H-NMR (300 MHz, DMSO-d₆) δ 10.4 (s, 1H), 10.1 (s, 1H), 8.50 (d, J=8.8 Hz, 1H), 7.20-7.48 (m, 5H), 6.38 (s, 1H), 6.25 (d, J=8.8 Hz, 1H), 3.74 (s, 3H), 2.37 (s, 3H).

MS (ESI): m/z=411.0220 [M+Na]⁺.

### Example 11

### Preparation of 5-(2,3-dihydroxybenzylidene)-1-methyl-3-(4-tolyl)-2-selenoxoimidazolidin-4-one (1abb, Compound 11)

Dioxane (1.0 mL) was added to the compound (75 mg, 0.28 mmol) obtained in step 3) of Example 10 and 2,3-dihydroxybenzaldehyde (42 mg, 0.31 mmol) and followed by stirring, piperidine (41 μl) and aluminum chloride (4 mg) were added, and the mixture was warmed at 90° C. for 5 h. After the reaction, the mixture was subjected to column separation to obtain the target compound (73 mg, 68%).

¹H-NMR (300 MHz, DMSO-d₆) δ 9.67 (br s, 1H), 9.18 (br s, 1H), 7.18-7.40 (m, 4H), 6.99 (s, 1H), 6.77-6.92 (m, 2H), 6.70-6.76 (m, 1H), 3.76 (s, 1H), 3.45 (s, 2H), 2.37 (s, 3H).

MS (ESI): m/z=389.0403 [M+H]⁺.

### Example 12

### Preparation of 5-(4-(dimethylamino)benzylidene)-1-methyl-3-(4-tolyl)-2-selenoxoimidazolidin-4-one (1abc, Compound 12)

Dioxane (0.6 mL) was added to the compound (53 mg, 0.2 mmol) obtained in step 3) of Example 10 and 4-dimethylaminobenzaldehyde (33 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (45 mg, 56%).

¹H-NMR (300 MHz, CDCl₃) δ 8.20 (d, J=8.5 Hz, 1H), 7.20-7.38 (m, 5H), 7.14 (s, 0.4H), 6.78 (s, 0.6H), 6.71 (d, J=8.5 Hz, 1H), 6.66 (d, J=8.3 Hz, 1H), 3.84 (s, 1.8H), 3.68 (s, 1.2H), 3.05 (s, 6H), 2.42 (s, 3H).

MS (ESI): m/z=400.0925 [M+H]⁺.

### Example 13

### Preparation of 5-(3,4-dihydroxybenzylidene)-1-methyl-3-(4-tolyl)-2-selenoxoimidazolidin-4-one (1abd, Compound 13)

Dioxane (0.6 mL) was added to the compound (53 mg, 0.2 mmol) obtained in step 3) of Example 10 and 3,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (31 mg, 40%).

¹H-NMR (300 MHz, DMSO-d₆) δ 9.79 (s, 1H), 9.23 (s, 1H), 7.95 (d, J=0.9 Hz, 1H), 7.53 (dd, J=8.5, 1.8 Hz, 1H), 7.22-7.36 (m, 4H), 7.09 (s, 1H), 6.77 (d, J=8.3 Hz, 1H), 3.76 (s, 3H), 2.37 (s, 3H).

MS (ESI): m/z=369.0401 [M+H]⁺.

### Example 14

### Preparation of 5-(3,5-dihydroxybenzylidene)-1-methyl-3-(4-tolyl)-2-selenoxoimidazolidin-4-one (1abe, Compound 14)

Dioxane (0.6 mL) was added to the compound (53 mg, 0.2 mmol) obtained in step 3) of Example 10 and 3,5-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (31 mg, 40%).

¹H-NMR (300 MHz, DMSO-d₆) δ 9.56 (s, 2H), 7.20-7.38 (m, 4H), 6.91 (m, 1H), 6.35 (d, J=1.4 Hz, 2H), 6.29 (d, J=1.9 Hz, 1H), 3.45 (s, 3H), 2.37 (s, 3H).

MS (ESI): m/z=389.0403 [M+H]⁺.

### Example 15

### Preparation of 1-methyl-5-(4-piperidin-1-yl)benzylidene-3-(4-tolyl)-2-selenoxoimidazolidin-4-one (1abh, Compound 15)

Dioxane (0.6 mL) was added to the compound (51 mg, 0.2 mmol) obtained in step 3) of Example 10 and 4-(piperidin-1-yl)benzaldehyde (42 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (77 mg, 83%).

¹H-NMR (300 MHz, CDCl₃) δ 8.16 (d, J=9.0 Hz, 1H), 7.15-7.40 (m, 5H), 7.12 (s, 0.4H), 6.78-6.96 (m, 2H), 6.76 (s, 0.6H), 3.84 (s, 1.8H), 3.65 (s, 1.2H), 3.25-3.50 (m, 4H), 2.41 (s, 3H), 1.50-1.90 (m, 5H), 1.20-1.37 (m, 1H).

MS (ESI): m/z=440.1237 [M+H]⁺.

### Example 16

### Preparation of 5-(4-bromobenzylidene)-1-methyl-2-selenoxo-3-(4-tolyl)imidazolidin-4-one (1abi, Compound 16)

Dioxane (0.6 mL) was added to the compound (53 mg, 0.2 mmol) obtained in step 3) of Example 10 and 4-bromobenzaldehyde (41 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (37 mg, 43%).

¹H-NMR (300 MHz, CDCl₃) δ 7.97 (d, J=8.6 Hz, 1H), 7.45-7.64 (m, 2H), 7.17-7.38 (m, 5H), 7.07 (s, 0.3H), 6.74 (s, 0.7H), 3.84 (s, 2H), 3.51 (s, 1H), 2.42 (s, 3H).

MS (ESI): m/z=456.9428 [M+Na]⁺.

### Example 17

### Preparation of 1-methyl-5-(4-pyrrolidin-1-yl)benzylidene-2-selenoxo-3-(4-tolyl)imidazolidin-4-one (1abk, Compound 17)

Dioxane (0.6 mL) was added to the compound (53 mg, 0.2 mmol) obtained in step 3) of Example 10 and 4-(pyrrolidin-1-yl)benzaldehyde (39 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (74 mg, 72%).

¹H-NMR (300 MHz, CDCl₃) δ 8.25 (d, J=8.6 Hz, 1H), 7.19-7.45 (m, 5H), 7.14 (s, 0.4H), 6.77 (s, 0.6H), 6.46-6.67 (m, 2H), 3.84 (s, 2H), 3.69 (s, 1H), 3.36 (br s, 4H), 1.89-2.14 (m, 4H).

MS (ESI): m/z=426.1083 [M+H]⁺.

### Example 18

### Preparation of 5-(4-hydroxybenzylidene)-1-methyl-2-selenoxo-3-(4-tolyl)imidazolidin-4-one (1abl, Compound 18)

Dioxane (0.6 mL) was added to the compound (53 mg, 0.2 mmol) obtained in step 3) of Example 10 and 4-hydroxybenzaldehyde (27 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (59 mg, 77%).

¹H-NMR (300 MHz, DMSO-d₆) δ 10.2 (br s, 1H), 8.15 (d, J=8.7 Hz, 2H), 7.19-7.38 (m, 4H), 7.18 (s, 0.9H), 7.01 (s, 0.1H), 6.80 (d, J=8.7 Hz, 2H), 3.76 (s, 2.7H), 3.50 (s, 0.3H), 2.37 (s, 4H), 1.90-2.05 (m, 3H).

MS (ESI): m/z=395.0270 [M+Na]⁺.

### Example 19

### Preparation of 1-methyl-5-(pyridin-3-ylmethylene)-2-selenoxo-3-(4-tolyl)imidazolidin-4-one (1abn, Compound 19)

Dioxane (0.6 mL) was added to the compound (53 mg, 0.2 mmol) obtained in step 3) of Example 10 and 3-pyridinecarboxaldehyde (24 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (39 mg, 55%).

¹H-NMR (300 MHz, DMSO-d₆) δ 9.05 (d, J=1.9 Hz, 0.4H), 8.77 (d, J=2.2 Hz, 0.6H), 8.62 (dd, J=4.8, 1.2 Hz, 0.6H), 8.50-8.58 (m, 0.8H), 8.00 (d, J=7.0 Hz, 0.6H), 7.40-7.53 (m, 1H), 7.24-7.37 (m, 4H), 7.23 (s, 0.4H), 7.09 (s, 0.6H), 3.77 (s, 1H), 3.42 (s, 2H), 2.38 (s, 2H), 2.37 (s, 1H).

MS (ESI): m/z=358.0457 [M+H]⁺.

### Example 20

### Preparation of 1-methyl-5-(4-(4-methylpiperazin-1-yl)benzylidene)-2-selenoxo-3-(4-tolyl)imidazolin-4-one (1abt, Compound 20)

Dioxane (0.6 mL) was added to the compound (53 mg, 0.2 mmol) obtained in step 3) of Example 10 and 4-(4-methylpiperazin-1-yl)benzaldehyde (49 mg, 0.21 mmol) and followed by stirring, piperidine (30 μl) and aluminum chloride (3 mg) were added, and the mixture was warmed at 90° C. for 5 h. After the reaction, the mixture was subjected to column separation to obtain the target compound (42 mg, 39%).

¹H-NMR (300 MHz, DMSO-d₆) δ 8.20 (d, J=9.0 Hz, 2H), 7.31 (d, J=8.3 Hz, 2H), 7.23 (d, J=8.3 Hz, 2H), 7.16 (s, 1H), 6.97 (d, J=9.2 Hz, 2H), 3.77 (s, 2.6H), 3.55 (s, 0.4H), 3.23-3.33 (m, 4H), 2.38-2.46 (m, 4H), 2.37 (s, 3H), 2.21 (s, 3H).

MS (EI): m/z=454.1269 [M]⁺.

### Example 21

### Preparation of 5-(4-(4-(2-hydroxyethyl)piperazin-1-yl)benzylidene)-1-methyl-2-selenoxo-3-(4-tolyl) imidazolin-4-one (1abu, Compound 21)

Dioxane (0.6 mL) was added to the compound (53 mg, 0.2 mmol) obtained in step 3) of Example 10 and 4-(4-(2-hydroxyethyl)piperazin-1-yl)benzaldehyde (49 mg, 0.21 mmol) and followed by stirring, piperidine (30 μl) and aluminum chloride (3 mg) were added, and the mixture was warmed at 90° C. for 5 h. After the reaction, the mixture was subjected to column separation to obtain the target compound (37 mg, 39%).

¹H-NMR (300 MHz, DMSO-d₆) δ 8.20 (d, J=8.9 Hz, 2H), 7.47 (d, J=8.9 Hz, 2H), 7.18-7.35 (m, 4H), 7.15 (s, 0.4H), 7.00 (s, 0.6H), 4.40-4.50 (m, 2H), 3.77 (s, 1.2H), 3.55 (s, 1.8H), 3.47-3.59 (m, 4H), 2.51-2.59 (m, 4H), 2.39-2.47 (m, 2H), 2.37 (s, 3H).

MS (EI): m/z=484.1373 [M]⁺.

### Example 22

### Preparation of 5-(4-((2-(2-hydroxyethoxy)ethyl)amino)benzylidene)-1-methyl-2-selenoxo-3-(4-tolyl)imidazolin-4-one (1abv, Compound 22)

Dioxane (0.5 mL) was added to the compound (43 mg, 0.16 mmol) obtained in step 3) of Example 10 and 4-((2-(2-hydroxyethoxy)ethyl)amino)benzaldehyde (33 mg, 0.17 mmol) and followed by stirring, piperidine (24 μl) and aluminum chloride (2.1 mg) were added, and the mixture was warmed at 90° C. for 5 h. After the reaction, the mixture was subjected to column separation to obtain the target compound (44 mg, 59%).

¹H-NMR (300 MHz, DMSO-d₆) δ 8.17 (d, J=8.9 Hz, 2H), 7.30 (d, J=8.3 Hz, 2H), 7.22 (d, J=8.3 Hz, 2H), 7.11 (s, 1H), 6.74 (t, J=5.5 Hz, 1H), 6.64 (d, J=8.9 Hz, 2H), 4.62 (t, J=5.3 Hz, 1H), 3.76 (s, 3H), 3.22-3.63 (m, 8H), 3.47-3.59 (m, 4H), 2.38 (s, 3H).

MS (ESI): m/z=459.1064 [M]⁺.

### Example 23

### Preparation of 1-methyl-5-(4-((2-morpholinoethyl)amino)benzylidene)-2-selenoxo-3-(4-tolyl)imidazolidin-4-one (1abw, Compound 23)

Dioxane (1.0 mL) was added to the compound (53 mg, 0.2 mmol) obtained in step 3) of Example 10 and 4-((2-morpholinoethyl)amino)benzaldehyde (53 mg, 0.2 mmol) and followed by stirring, piperidine (40 μl) and aluminum chloride (5.3 mg) were added, and the mixture was warmed at 90° C. for 5 h. After the reaction, the mixture was subjected to column separation to obtain the target compound (37 mg, 38%).

¹H-NMR (300 MHz, DMSO-d₆) δ 8.17 (d, J=8.9 Hz, 2H), 7.30 (d, J=8.2 Hz, 2H), 7.22 (d, J=8.2 Hz, 2H), 7.11 (s, 1H), 6.96 (s, 0.2H), 6.63 (d, J=8.9 Hz, 2H), 6.50-6.70 (m, 1H), 3.76 (s, 2.4H), 3.53-3.64 (m, 4H), 3.42 (s, 0.6H), 3.16-3.26 (m, 2H), 2.39-2.45 (m, 4H), 2.38 (s, 3H).

MS (EI): m/z=484.1380 [M]⁺.

### Example 24

### Preparation of 5-(2,4-dihydroxybenzylidene)-3-(4-ethylphenyl)-1-methyl-2-selenoxoimidazolidin-4-one (1aca, Compound 24)

### 1) Synthesis of N-(4-ethylphenyl)formamide

After 4-ethylaniline (26 g, 215 mmol) was added to formic acid (30 g, 644 mmol), the reaction was conducted under reflux at 100° C. for 4 hours and terminated. After ethyl acetate was added to the reaction mixture for dilution, the organic layer was washed with water, 0.1 N aqueous hydrochloric acid solution, saturated sodium hydrogen carbonate solution, and brine. The organic layer was dried over anhydrous sodium sulfate, filtered, and concentrated under reduced pressure to obtain the target compound (28 g, 87%).

¹H-NMR (300 MHz, CDCl₃) δ 8.64 (d, J=11.4 Hz, 0.6H), 8.20-8.55 (br s, 0.4H), 8.34 (s, 0.6H), 7.60 (br s, 0.4H), 7.45 (d, J=8.4 Hz, 1H), 7.17 (t, J=8.1 Hz, 2H), 7.02 (d, J=8.9 Hz, 1H), 2.55-2.70 (m, 2H), 1.15-1.30 (m, 3H).

MS (EI): m/z=149 [M+H]⁺.

### 2) Synthesis of 4-ethylphenyl isoselenocyanate

After N-(4-ethylphenyl)formylformamide (4.92 g, 33 mmol) was dissolved in methylene chloride (145 mL), triethylamine (20 mL, 4.30 mmol) and 4A MS (660 mg) were added. After triphosgene (5.3 g, 18 mmol) was dissolved in methylene chloride (25 mL), the solution was slowly added dropwise over 1 hour. The reaction mixture was refluxed for 4 h, then selenium (5.20 g, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (2.8 g, 40%).

¹H-NMR (300 MHz, CDCl₃) δ 7.19 (s, 4H), 2.64 (q, J=7.6 Hz, 2H), 1.22 (t, J=7.6 Hz, 3H).

MS (EI): m/z=211 [M+H]⁺.

### 3) Synthesis of 3-(4-ethylphenyl)-1-methyl-2-selenoxoimidazolidin-4-one

Dioxane (170 mL) and sarcosine (346 mg, 3.89 mmol) were added to the compound (817 mg, 3.89 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (1.02 g, 92%).

¹H-NMR (300 MHz, CDCl₃) δ 7.33 (d, J=8.3 Hz, 2H), 7.22 (d, J=8.3 Hz, 2H), 4.06 (s, 2H), 3.50 (s, 3H), 2.71 (q, J=7.6 Hz, 2H), 1.27 (t, J=7.6 Hz, 3H).

MS (EI): m/z=282 [M+H]⁺.

### 4) Synthesis of 5-(2,4-dihydroxybenzylidene)-3-(4-ethylphenyl)-1-methyl-2-selenoxoimidazolidin-4-one (1aca)

Dioxane (0.6 mL) was added to the compound (53 mg, 0.2 mmol) obtained in step 3) and 2,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (47 mg, 61%).

¹H-NMR (300 MHz, DMSO-d₆) δ 10.4 (s, 1H), 10.1 (s, 1H), 8.49 (d, J=8.8 Hz, 1H), 7.21-7.40 (m, 5H), 6.38 (d, J=2.3 Hz, 1H), 6.24 (dd, J=8.9, 2.3 Hz, 1H), 3.74 (s, 3H), 2.67 (q, J=7.5 Hz, 2H), 1.23 (t, J=7.5 Hz, 3H).

MS (ESI): m/z=403.0559 [M+H]⁺.

### Example 25

### Preparation of 5-(4-(dimethylamino)benzylidene)-3-(4-ethylphenyl)-1-methyl-2-selenoxoimidazolidin-4-one (1acc, Compound 25)

Dioxane (0.6 mL) was added to the compound (57 mg, 0.20 mmol) obtained in step 3) of Example 24 and 4-dimethylaminobenzaldehyde (36 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (41 mg, 50%).

¹H-NMR (300 MHz, DMSO-d₆) δ 8.20 (d, J=9.1 Hz, 1H), 7.20-7.42 (m, 5H), 7.13 (s, 0.3H), 6.77 (s, 0.7H), 6.71 (d, J=8.9 Hz, 0.3H), 6.65 (d, J=9.1 Hz, 0.7H), 3.84 (s, 3H), 3.67 (s, 1.5H), 2.72 (q, J=7.6 Hz, 2H), 1.28 (t, J=7.6 Hz, 3H).

MS (ESI): m/z=414.1083 [M+H]⁺.

### Example 26

### Preparation of 5-(3,4-dihydroxybenzylidene)-3-(4-ethylphenyl)-1-methyl-2-selenoxoimidazolidin-4-one (1acd, Compound 26)

Dioxane (0.6 mL) was added to the compound (57 mg, 0.20 mmol) obtained in step 3) of Example 24 and 4-dimethylaminobenzaldehyde (36 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (41 mg, 50%).

¹H-NMR (300 MHz, DMSO-d₆) δ 9.79 (br s, 1H), 9.24 (br s, 1H), 7.96 (d, J=1.9 Hz, 1H), 7.54 (dd, J=8.5, 1.8 Hz, 1H), 7.22-7.40 (m, 4H), 7.09 (s, 1H), 6.77 (d, J=8.3 Hz, 1H), 3.76 (s, 3H), 2.67 (q, J=7.6 Hz, 2H), 1.23 (t, J=7.6 Hz, 3H).

MS (EI): m/z=402.0484 [M]⁺.

### Example 27

### Preparation of 5-(3,5-dihydroxybenzylidene)-3-(4-ethylphenyl)-1-methyl-2-selenoxoimidazolidin-4-one (1ace, Compound 27)

Dioxane (0.6 mL) was added to the compound (57 mg, 0.20 mmol) obtained in step 3) of Example 24 and 3,5-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (40 mg, 50%).

¹H-NMR (300 MHz, DMSO-d⁶) δ δ 9.56 (s, 1H), 9.40 (s, 1H), 7.23-7.44 (m, 4H), 7.07 (d, J=8.2 Hz, 1H), 7.01 (s, 0.4H), 6.91 (s, 0.6H), 6.35 (d, J=1.6 Hz, 1H), 6.20-6.32 (m, 1H), 3.75 (s, 1.2H), 3.45 (s, 1.8H), 2.67 (q, J=7.6 Hz, 2H), 1.23 (dt, J=7.6, 1.8 Hz, 3H).

MS (ESI): m/z=403.0560 [M+H]⁺.

### Example 28

### Preparation of 3-(4-ethylphenyl)-1-methyl-5-(4-(piperidin-1-yl)benzylidene)-2-selenoxoimidazolidin-4-one (1ach, Compound 28)

Dioxane (0.6 mL) was added to the compound (57 mg, 0.20 mmol) obtained in step 3) of Example 24 and 4-(piperidinyl)benzaldehyde (42 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (55 mg, 61%).

¹H-NMR (300 MHz, CDCl₃) δ 8.16 (d, J=9.0 Hz, 2H), 7.20-7.48 (m, 4H), 6.82 (d, J=9.0 Hz, 2H), 6.75 (s, 1H), 3.83 (s, 3H), 3.51 (s, 0.6H), 3.26-3.50 (m, 4H), 2.71 (q, J=7.6 Hz, 2H), 1.65 (br s, 6H), 1.27 (t, J=7.6 Hz, 3H).

MS (ESI): m/z=454.1394 [M+H]⁺.

### Example 29

### Preparation of 5-(4-bromobenzylidene)-3-(4-ethylphenyl)-1-methyl-2-selenoxoimidazolidin-4-one (1aci, Compound 29)

Dioxane (0.6 mL) was added to the compound (57 mg, 0.20 mmol) obtained in step 3) of Example 24 and 4-bromobenzaldehyde (41 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (60 mg, 67%).

¹H-NMR (300 MHz, CDCl₃) δ 7.97 (d, J=8.6 Hz, 2H), 7.45-7.62 (m, 2H), 7.21-7.42 (m, 4H), 7.07 (s, 0.2H), 6.74 (s, 0.8H), 3.83 (s, 2.4H), 3.51 (s, 0.6H), 2.72 (q, J=7.6 Hz, 2H), 1.27 (t, J=7.6 Hz, 3H).

MS (ESI): m/z=470.9584 [M+Na]⁺.

### Example 30

### Preparation of 3-(4-ethylphenyl)-1-methyl-5-(4-(pyrrolidin-1-yl)benzylidene)-2-selenoxoimidazolidin-4-one (1ack, Compound 30)

Dioxane (0.6 mL) was added to the compound (57 mg, 0.20 mmol) obtained in step 3) of Example 24 and 4-(pyrrolidinyl)benzaldehyde (39 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (63 mg, 72%).

¹H-NMR (300 MHz, DMSO-d₆) δ 8.24 (d, J=8.7 Hz, 2H), 7.33 (d, J=8.1 Hz, 2H), 7.25 (d, J=8.3 Hz, 2H), 7.14 (s, 1H), 6.58 (d, J=8.9 Hz, 2H), 3.77 (s, 3H), 2.67 (q, J=7.6 Hz, 2H), 1.23 (t, J=7.6 Hz, 3H).

MS (ESI): m/z=440.1237 [M+H]⁺.

### Example 31

### Preparation of 3-(4-ethylphenyl)-1-methyl-5-(pyridin-3-ylmethylene)-2-selenoxoimidazolidin-4-one (1acn, Compound 31)

Dioxane (0.6 mL) was added to the compound (57 mg, 0.20 mmol) obtained in step 3) of Example 24 and 3-pyridinecarboxaldehyde (24 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (45 mg, 61%).

¹H-NMR (300 MHz, DMSO-d₆) δ 9.05 (s, 0.3H), 8.77 (s, 0.7H), 8.62, 8.55 (each d, J=4.8, 5.4 Hz, 1.3H), 7.99 (d, J=7.9 Hz, 0.7H), 7.40-7.57 (m, 1.3H), 7.28-7.39 (m, 4H), 7.27 (s, 0.3H), 7.09 (s, 0.7H), 3.77 (s, 0.9H), 3.42 (s, 2.1H), 2.68 (q, J=7.6 Hz, 2H), 1.23 (t, J=7.6 Hz, 3H).

MS (ESI): m/z=375.0612 [M+H]⁺.

### Example 32

### Preparation of 3-(4-ethylphenyl)-1-methyl-5-(4-morpholidinobenzylidene)-2-selenoxoimidazolidin-4-one (1acs, Compound 32)

Dioxane (0.8 mL) was added to the compound (57 mg, 0.20 mmol) obtained in step 3) of Example 24 and 4-morpholinobenzaldehyde (38 mg, 0.22 mmol) and followed by stirring, piperidine (79 μl) and aluminum chloride (6 mg) were added, and the mixture was warmed at 90° C. for 5 h. After the reaction, the mixture was subjected to column separation to obtain the target compound (56 mg, 62%).

¹H-NMR (300 MHz, CD₃CN) δ 8.20 (d, J=8.9 Hz, 1.2H), 7.30-7.50 (m, 2.8H), 7.17-7.29 (m, 2H), 6.86-7.05 (m, 3H), 3.70-3.85 (m, 4H), 3.77 (s, 1.7H), 3.56 (s, 1.3H), 3.15-3.35 (m, 4H), 2.74 (q, J=7.6 Hz, 2H), 1.28 (t, J=7.6 Hz, 3H).

MS (ESI): m/z=455.1115 [M]⁺.

### Example 33

### Preparation of 3-(4-ethylphenyl)-5-(4-(4-(2-hydroxyethyl)piperazin-1-yl)benzylidene)-1-methyl-2-selenoxoimidazolidin-4-one (1acu, Compound 33)

Dioxane (0.6 mL) was added to the compound (57 mg, 0.20 mmol) obtained in step 3) of Example 24 and 4-(4-(2-hydroxyethyl)piperazin-1-yl)benzaldehyde (49 mg, 0.21 mmol) and followed by stirring, piperidine (30 μl) and aluminum chloride (3 mg) were added, and the mixture was warmed at 90° C. for 5 h. After the reaction, the mixture was subjected to column separation to obtain the target compound (51 mg, 52%).

¹H-NMR (300 MHz, CD₃CN) δ 8.16 (d, J=9.0 Hz, 1H), 7.30-7.50 (m, 4H), 7.26 (s, 0.7H), 7.23 (s, 0.3H), 6.90-7.10 (m, 3H), 3.75 (s, 2H), 3.56-3.65 (m, 2H), 3.55 (s, 1H), 3.20-3.40 (m, 4H), 2.72 (d, J=7.6 Hz, 2H), 2.54-2.67 (m, 4H), 2.50 (d, J=5.6 Hz, 2H), 1.26 (t, J=7.6 Hz, 3H).

MS (ESI): m/z=498.1538 [M]⁺.

### Example 34

### Preparation of 3-(4-ethylphenyl)-5-(4-((2-(2-fluoroethoxy)ethyl)amino)benzylidene)-1-methyl-2-selenoxoimidazolidin-4-one (1acy, Compound 34)

Dioxane (0.6 mL) was added to the compound (57 mg, 0.20 mmol) obtained in step 3) of Example 24 and 4-(4-(2-fluoroethoxy)ethyl)aminobenzaldehyde (44 mg, 0.21 mmol) and followed by stirring, piperidine (30 μl) and aluminum chloride (3 mg) were added, and the mixture was warmed at 90° C. for 5 h. After the reaction, the mixture was subjected to column separation to obtain the target compound (68 mg, 72%).

¹H-NMR (300 MHz, CD₃CN) δ 8.14 (d, J=8.9 Hz, 2H), 7.35 (d, J=8.3 Hz, 2H), 7.25 (d, J=8.3 Hz, 2H), 6.94 (s, 1H), 6.65 (d, J=8.9 Hz, 2H), 5.28 (br s, 1H), 4.54-4.57 (m, 1H), 4.39-4.51 (m, 1H), 3.76 (s, 3H), 3.52-3.75 (m, 4H), 3.33 (q, J=5.5 Hz, 2H), 2.72 (q, J=7.6 Hz, 2H), 1.26 (t, J=7.6 Hz, 3H).

MS (ESI): m/z=475.1176 [M]⁺.

### Example 35

### Preparation of 3-(4-chlorophenyl)-5-(2,4-dihydroxybenzylidene)-1-methyl-2-selenoxoimidazolidin-4-one (1ada, Compound 35)

### 1) Synthesis of N-4-chlorophenylformamide

To formic acid (30 g, 644 mmol), 4-chloroaniline (27 g, 215 mmol) was added. The reaction was conducted under reflux at 100° C. for 4 hours and terminated. After ethyl acetate was added to the reaction mixture for dilution, zinc oxide was removed by filtration. The organic layer was washed with water, 0.1 N aqueous hydrochloric acid solution, saturated sodium hydrogen carbonate solution, and brine. The organic layer was dried over anhydrous sodium sulfate, filtered, and concentrated under reduced pressure to obtain the target compound (27 g, 82%).

¹H-NMR (300 MHz, CDCl₃) δ 8.64 (d, J=11.3 Hz, 0.5H), 8.37 (d, J=1.5 Hz, 0.5H), 7.79 (br s, 0.5H), 7.50 (d, J=9.6 Hz, 1H), 7.19-7.36 (m, 2.5H), 7.02 (d, J=9.6 Hz, 1H).

MS (EI): m/z=155 [M]⁺.

### 2) Synthesis of 4-chlorophenyl isoselenocyanate

After N-4-chlorophenylformamide (5.20 g, 33 mmol) was dissolved in methylene chloride (145 mL), triethylamine (20 mL, 4.3 mmol) and 4A MS (660 mg) were added. After triphosgene (5.3 g, 18 mmol) was dissolved in methylene chloride (25 mL), the solution was slowly added dropwise over 1 hour. The reaction mixture was refluxed for 4 h, then selenium (5.20 g, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (4.9 g, 68%).

¹H-NMR (300 MHz, CDCl₃) δ 7.35 (d, J=8.5 Hz, 2H), 7.23 (d, J=8.5 Hz, 2H).

MS (EI): m/z=217 [M+H]⁺.

### 3) Synthesis of 1-methyl-3-(4-chlorophenyl)-2-selenoxoimidazolidin-4-one

Dioxane (170 mL) and sarcosine (346 mg, 3.89 mmol) were added to the compound (842 mg, 3.89 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (1.06 g, 95%).

¹H-NMR (300 MHz, CDCl₃) δ 7.48 (d, J=6.7 Hz, 2H), 7.29 (d, J=6.7 Hz, 2H), 4.08 (s, 2H), 3.51 (s, 3H).

MS (EI): m/z=268 [M+H]⁺.

### 4) Synthesis of 3-(4-chlorophenyl)-5-(2,4-dihydroxybenzylidene)-1-methyl-2-selenoxoimidazolidin-4-one (1ada)

Dioxane (0.6 mL) was added to the compound (58 mg, 0.2 mmol) obtained in step 3) and 2,4-dihydroxybenzaldehyde (33 mg, 0.24 mmol) and followed by stirring, piperidine (30 μl) and aluminum chloride (3 mg) were added, and the mixture was warmed at 90° C. for 5 h. After the reaction, the mixture was subjected to column separation to obtain the target compound (34 mg, 41%).

¹H-NMR (300 MHz, DMSO-d₆) δ 10.4 (br s, 1H), 10.1 (br s, 1H), 8.49 (d, J=8.9 Hz, 1H), 7.58 (d, J=8.6 Hz, 2H), 7.43 (d, J=8.6 Hz, 2H), 7.32 (s, 1H), 6.38 (d, J=2.3 Hz, 1H), 6.26 (dd, J=8.8, 2.3 Hz, 1H), 3.74 (s, 3H).

MS (EI): m/z=407.9781 [M]⁺.

### Example 36

### Preparation of 3-(4-chlorophenyl)-5-(2,3-dihydroxybenzylidene)-1-methyl-2-selenoxoimidazolidin-4-one (1adb, Compound 36)

Dioxane (0.6 mL) was added to the compound (58 mg, 0.2 mmol) obtained in step 3) of Example 35 and 2,3-dihydroxybenzaldehyde (33 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (35 mg, 43%).

¹H-NMR (300 MHz, CDCl₃) δ 7.47 (d, J=8.7 Hz, 2H), 7.32 (d, J=8.6 Hz, 2H), 7.15 (s, 0.6H), 7.01 (s, 0.7H), 7.11 (s, 0.3H), 6.73-7.00 (m, 3H), 3.87 (s, 0.8H), 3.52 (s, 2.2H).

MS (ESI): m/z=430.9675 [M+Na]⁺.

### Example 37

### Preparation of 3-(4-chlorophenyl)-5-(3,4-dihydroxybenzylidene)-1-methyl-2-selenoxoimidazolidin-4-one (1add, Compound 37)

Dioxane (1.0 mL) was added to the compound (80 mg, 0.28 mmol) obtained in step 3) of Example 35 and 3,4-dihydroxybenzaldehyde (42 mg, 0.31 mmol) and followed by stirring, piperidine (41 μl) and aluminum chloride (4 mg) were added, and the mixture was warmed at 90° C. for 5 h. After the reaction, the mixture was subjected to column separation to obtain the target compound (53 mg, 46%).

¹H-NMR (300 MHz, CDCl₃) δ 9.69 (br s, 2H), 7.96 (s, 1H), 7.58 (d, J=8.5 Hz, 2H), 7.53 (d, J=8.4 Hz, 1H), 7.43 (d, J=8.5 Hz, 2H), 7.10 (s, 1H), 6.78 (d, J=8.3 Hz, 1H), 3.75 (s, 3H).

MS (ESI): m/z=408.9860 [M+H]⁺.

### Example 38

### Preparation of 3-(4-chlorophenyl)-5-(3,5-dihydroxybenzylidene)-1-methyl-2-selenoxoimidazolidin-4-one (1ade, Compound 38)

Dioxane (1.0 mL) was added to the compound (80 mg, 0.28 mmol) obtained in step 3) of Example 35 and 3,5-dihydroxybenzaldehyde (42 mg, 0.31 mmol) and followed by stirring, piperidine (41 μl) and aluminum chloride (4 mg) were added, and the mixture was warmed at 90° C. for 5 h. After the reaction, the mixture was subjected to column separation to obtain the target compound (52 mg, 46%).

¹H-NMR (300 MHz, CDCl₃) δ 9.57 (s, 1H), 9.42 (s, 1H), 7.54-7.69 (m, 2H), 7.38-7.50 (m, 2H), 7.07 (d, J=1.9 Hz, 1H), 7.05 (s, 0.4H), 6.93 (s, 0.6H), 6.35 (d, J=1.7 Hz, 1H), 6.20-6.33 (m, 1H), 3.75 (s, 1.8H), 3.56 (s, 1.2H).

MS (EI): m/z=407.9777 [M]⁺.

### Example 39

### Preparation of 3-(4-chlorophenyl)-1-methyl-5-(pyridin-3-ylmethylene)-2-selenoxoimidazolidin-4-one (1adn, Compound 39)

Dioxane (0.6 mL) was added to the compound (58 mg, 0.2 mmol) obtained in step 3) of Example 35 and 3-pyridinecarboxaldehyde (24 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (41 mg, 55%).

¹H-NMR (300 MHz, DMSO-d₆) δ 9.05 (s, 0.55H), 8.77 (s, 0.45H), 8.46-8.70 (m, 1.5H), 7.99 (d, J=7.7 Hz, 0.5H), 7.39-7.77 (m, 5H), 6.50-6.67 (m, 2H), 3.78 (s, 1.8H), 3.60 (s, 1.2H), 3.34 (br s, 4H), 1.90-2.05 (m, 4H).

MS (ESI): m/z=377.9910 [M+H]⁺.

### Example 40

### Preparation of 3-(3-chlorophenyl)-5-(2,3-dihydroxybenzylidene)-1-methyl-2-selenoxoimidazolidin-4-one (1aeb, Compound 40)

### 1) Synthesis of N-3-chlorophenylformamide

To formic acid (30 g, 644 mmol), 3-chloroaniline (27 g, 215 mmol) was added. The reaction was conducted under reflux at 100° C. for 4 hours and terminated. After ethyl acetate was added to the reaction mixture for dilution, zinc oxide was removed by filtration. The organic layer was washed with water, 0.1 N aqueous hydrochloric acid solution, saturated sodium hydrogen carbonate solution, and brine. The organic layer was dried over anhydrous sodium sulfate, filtered, and concentrated under reduced pressure to obtain the target compound (33 g, 100%).

¹H-NMR (300 MHz, CDCl₃) δ 8.70 (d, J=11.1 Hz, 0.5H), 8.38 (s, 0.5H), 7.67 (s, 0.5H), 7.67 (t, J=1.9 Hz, 1H), 6.94-7.46 (m, 3H).

MS (EI): m/z=155 [M]⁺.

### 2) Synthesis of 3-chlorophenyl isoselenocyanate

After N-3-chlorophenylformamide (5.20 g, 33 mmol) was dissolved in methylene chloride (145 mL), triethylamine (20 mL, 4.30 mmol) and 4A MS (660 mg) were added. After triphosgene (5.30 g, 18 mmol) was dissolved in methylene chloride (25 mL), the solution was slowly added dropwise over 1 hour. The reaction mixture was refluxed for 4 h, then selenium (5.20 g, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (4.9 g, 68%).

¹H-NMR (300 MHz, CDCl₃) δ 7.25-7.37 (m, 3H), 7.12-7.23 (m, 1H).

MS (EI): m/z=217 [M+H]⁺.

### 3) Synthesis of 1-methyl-3-(3-chlorophenyl)-2-selenoxoimidazolidin-4-one

Dioxane (170 mL) and sarcosine (346 mg, 3.89 mmol) were added to the compound (842 mg, 3.89 mmol) obtained in step 2), and heating was performed at 110° C. for 5 h. After the reaction, the reaction mixture was cooled to room temperature and concentrated under reduced pressure, and the resulting residue was subjected to column separation to obtain the target compound (951 mg, 85%).

¹H-NMR (300 MHz, CDCl₃) δ 7.40-7.49 (m, 2H), 7.35 (s, 1H), 4.08 (s, 2H), 3.51 (s, 3H).

MS (EI): m/z=268 [M+H]⁺.

### 4) Synthesis of 3-(3-chlorophenyl)-5-(2,3-dihydroxybenzylidene)-1-methyl-2-selenoxoimidazolidin-4-one (1aeb)

Dioxane (0.6 mL) was added to the compound (80 mg, 0.28 mmol) obtained in step 3) and 2,3-dihydroxybenzaldehyde (42 mg, 0.31 mmol) and followed by stirring, piperidine (30 μl) and aluminum chloride (3 mg) were added, and the mixture was warmed at 90° C. for 5 h. After the reaction, the mixture was subjected to column separation to obtain the target compound (34 mg, 30%).

¹H-NMR (300 MHz, DMSO-d₆) δ 9.57 (s, 1H), 9.41 (s, 1H), 7.50-7.68 (m, 3H), 7.07 (d, J=2.0 Hz, 1H), 7.05 (s, 0.6H), 6.94 (s, 0.4H), 6.65-6.75 (m, 0.3H), 6.35 (d, J=1.6 Hz, 0.7H), 3.75 (s, 2H), 3.46 (1H).

MS (ESI): m/z=408.9853 [M+H]⁺.

### Example 41

### Preparation of 5-(2,4-dihydroxybenzylidene)-1-methyl-3-phenethyl-2-selenoxoimidazolidin-4-one (1aha, Compound 41)

### 1) Synthesis of N-phenethylformamide

After phenethylamine (26 g, 215 mmol) was added to formic acid (30 g, 644 mmol), the reaction was conducted under reflux at 100° C. for 4 hours and terminated. After ethyl acetate was added to the reaction mixture for dilution, the organic layer was washed with water, 0.1 N aqueous hydrochloric acid solution, saturated sodium hydrogen carbonate solution, and brine. The organic layer was dried over anhydrous sodium sulfate, filtered, and concentrated under reduced pressure to obtain the target compound (25 g, 79%).

¹H-NMR (300 MHz, CDCl₃) δ 8.13 (s, 0.8H), 7.93 (d, J=11.9 Hz, 0.2H), 7.00-7.60 (m, 5H), 5.60 (br s, 1H), 4.00-4.30 (m, 0.6H), 3.40-3.70 (m, 2H), 2.80-3.00 (m, 2H).

MS (EI): m/z=149 [M]⁺.

### 2) Synthesis of N-phenethyl isoselenocyanate

After N-(phenethyl)formylformamide (4.92 g, 33 mmol) was dissolved in methylene chloride (145 mL), triethylamine (20 mL, 4.30 mmol) and 4A MS (660 mg) were added. After triphosgene (5.30 g, 18 mmol) was dissolved in methylene chloride (25 mL), the solution was slowly added dropwise over 1 hour. The reaction mixture was refluxed for 4 h, then selenium (5.20 g, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (2.5 g, 36%).

¹H-NMR (300 MHz, CDCl₃) δ 7.00-7.40 (m, 5H), 3.52-3.70 (m, 2H), 2.80-3.10 (m, 2H)

MS (ESI): m/z=211 [M+H]⁺.

### 3) Synthesis of 1-methyl-3-phenethyl-2-selenoxoimidazolidin-4-one

Dioxane (170 mL) and sarcosine (346 mg, 3.89 mmol) were added to the compound (817 mg, 3.89 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (746 mg, 68%).

¹H-NMR (300 MHz, CDCl₃) δ 7.10-7.40 (m, 5H), 7.22 (d, J=8.3 Hz, 2H), 4.00-4.20 (m, 2H), 3.84 (s, 2H), 3.43 (s, 3H), 2.90-3.15 (m, 2H).

MS (EI): m/z=281 [M]⁺.

### 4) Synthesis of 5-(2,4-dihydroxybenzylidene)-1-methyl-3-phenethyl-2-selenoxoimidazolidin-4-one (1aha)

Dioxane (0.6 mL) was added to the compound (53 mg, 0.2 mmol) obtained in step 3) and 2,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (47 mg, 61%).

¹H-NMR (300 MHz, DMSO-d₆) δ 10.4 (br s, 1H), 10.2 (br s, 1H), 8.52 (d, J=8.8 Hz, 1H), 7.10-7.40 (m, 6H), 6.37 (s, 1H), 6.28 (d, J=9.1 Hz, 1H), 4.09 (br s, 2H), 3.66 (s, 3H), 2.93 (br s, 2H).

MS (ESI): m/z=425.0379 [M+Na]⁺.

### Example 42

### Preparation of 5-(2,3-dihydroxybenzylidene)-1-methyl-3-phenethyl-2-selenoxoimidazolidin-4-one (1ahb, Compound 42)

Dioxane (0.6 mL) was added to the compound (57 mg, 0.20 mmol) obtained in step 3) of Example 41 and 2,3-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (39 mg, 48%).

¹H-NMR (300 MHz, DMSO-d₆) δ 9.59 (br s, 2H), 7.77 (d, J=9.1 Hz, 1H), 7.15-7.40 (m, 5H), 6.60-7.10 (m, 3H), 4.05-4.20 (m, 2H), 3.73 (s, 3H), 2.93-3.04 (m, 2H).

MS (ESI): m/z=425.0378 [M+Na]⁺.

### Example 43

### 5-(4-(dimethylamino)benzylidene)-1-methyl-3-phenethyl-2-selenoxoimidazolidin-4-one (1ahc, Compound 43)

Dioxane (0.6 mL) was added to the compound (57 mg, 0.20 mmol) obtained in step 3) of Example 41 and 4-dimethylaminobenzaldehyde (36 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (40 mg, 49%).

¹H-NMR (300 MHz, DMSO-d₆) δ 8.22 (d, J=8.7 Hz, 2H), 7.43 (d, J=5.6 Hz, 2H), 7.15-7.38 (m, 5H), 7.08 (s, 0.6H), 6.93 (s, 0.4H), 4.00-4.15 (m, 2H), 3.69 (s, 3H), 2.99 (s, 3H), 2.96 (s, 3H), 2.74-2.95 (m, 2H).

MS (ESI): m/z=414.1085 [M+H]⁺.

### Example 44

### Preparation of 5-(3,5-dihydroxybenzylidene)-1-methyl-3-phenethyl-2-selenoxoimidazolidin-4-one (1ahe, Compound 44)

Dioxane (0.6 mL) was added to the compound (57 mg, 0.20 mmol) obtained in step 3) of Example A and 3,5-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (52 mg, 60%).

¹H-NMR (300 MHz, DMSO-d₆) δ 9.46 (s, 2H), 7.15-7.40 (m, 5H), 7.08 (s, 2H), 6.98 (s, 1H), 6.33 (d, J=1.9 Hz, 1H), 4.10 (t, J=7.7 Hz, 2H), 3.68 (s, 3H), 2.94 (t, J=7.7 Hz, 2H).

MS (ESI): m/z=425.0379 [M+Na]⁺.

### Example 45

### Preparation of 1-methyl-3-phenethyl-5-(4-(piperidin-1-yl)benzylidene)-2-selenoxoimidazolidin-4-one (1abh, Compound 45)

Dioxane (0.6 mL) was added to the compound (57 mg, 0.20 mmol) obtained in step 3) of Example 41 and 4-(piperidinyl)benzaldehyde (42 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (79 mg, 87%).

¹H-NMR (300 MHz, CDCl₃) δ 8.13 (d, J=9.0 Hz, 2H), 7.18-7.43 (m, 5H), 6.87 (d, J=9.0 Hz, 2H), 6.68 (s, 1H), 4.18-4.34 (m, 2H), 3.75 (s, 2.4H), 3.51 (s, 0.6H), 3.25-3.45 (m, 4H), 2.94-3.14 (m, 2H), 1.60-1.75 (m, 6H).

MS (ESI): m/z=454.1395 [M+H]⁺.

### Example 46

### Preparation of 5-(4-chlorobenzylidene)-1-methyl-3-phenethyl-2-selenoxoimidazolidin-4-one (1ahj, Compound 46)

Dioxane (0.6 mL) was added to the compound (57 mg, 0.20 mmol) obtained in step 3) of Example 41 and 4-chlorobenzaldehyde (24 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (30 mg, 54%).

H-NMR (300 MHz, CDCl₃) δ 8.11 (dd, J=4.2, 1.5 Hz, 0.8H), 7.20-7.40 (m, 5H), 6.94 (t, J=1.9 Hz, 0.8H), 6.88 (t, J=1.9 Hz, 0.2H), 6.58 (s, 0.8H), 6.51-6.56 (m, 0.2H), 6.26-6.73 (m, 0.8H), 6.20-6.26 (m, 0.2H), 4.17-4.32 (m, 2H), 3.77 (s, 2H), 3.76 (s, 3H), 3.71 (s, 1H), 2.98-3.10 (m, 2H).

### Example 47

### Preparation of 1-methyl-3-phenethyl-5-(4-(pyrrolidin-1-yl)benzylidene)-2-selenoxoimidazolidin-4-one (1ahk, Compound 47)

Dioxane (0.6 mL) was added to the compound (57 mg, 0.20 mmol) obtained in step 3) of Example 41 and 4-(pyrrolidinyl)benzaldehyde (39 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (46 mg, 53%).

¹H-NMR (300 MHz, CDCl₃) δ 8.17 (d, J=8.9 Hz, 2H), 7.15-7.40 (m, 5H), 6.69 (s, 1H), 6.56 (d, J=8.9 Hz, 2H), 4.17-4.35 (m, 2H), 3.76 (s, 3H), 3.38 (t, J=6.6 Hz, 4H), 2.96-3.14 (m, 2H), 2.04 (t, J=6.6 Hz, 4H).

MS (ESI): m/z=462.1048 [M+Na]⁺.

### Example 48

### Preparation of 5-(4-hydroxybenzylidene)-1-methyl-3-phenethyl-2-selenoxoimidazolidin-4-one (1ahl, Compound 48)

Dioxane (0.6 mL) was added to the compound (57 mg, 0.20 mmol) obtained in step 3) of Example 41 and 4-hydroxybenzaldehyde (22 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (56 mg, 78%).

H-NMR (300 MHz, CDCl₃) δ 7.56 (d, J=1.5 Hz, 1H), 7.29-7.38 (m, 5H), 7.18-7.29 (m, 1H), 6.85 (d, J=3.5 Hz, 1H), 6.70 (s, 1H), 6.57 (dd, J=3.5, 1.8 Hz, 1H), 4.20-4.29 (m, 2H), 4.06 (s, 3H), 3.00-3.09 (m, 2H).

### Example 49

### Preparation of 5-(3-hydroxybenzylidene)-1-methyl-3-phenethyl-2-selenoxoimidazolidin-4-one (1ahm, Compound 49)

Dioxane (0.6 mL) was added to the compound (57 mg, 0.20 mmol) obtained in step 3) of Example 41 and 3-hydroxybenzaldehyde (26 mg, 0.21 mmol) and followed by stirring, piperidine (30 μl) and aluminum chloride (3 mg) were added, and the mixture was warmed at 90° C. for 5 h. After the reaction, the mixture was subjected to column separation to obtain the target compound (61 mg, 79%).

¹H-NMR (300 MHz, CD₃CN) δ 7.15-7.40 (m, 7H), 6.93 (s, 1H), 6.82-6.91 (m, 2H), 4.10-4.19 (m, 2H), 3.67 (s, 1H), 3.36 (s, 2H), 2.96-3.60 (m, 2H).

MS (ESI): m/z=396.0535 [M]⁺.

### Example 50

### Preparation of 1-methyl-5-(naphthalen-2-ylmethylene)-3-phenethyl-2-selenoxoimidazolidin-4-one (1ahp, Compound 50)

Dioxane (0.6 mL) was added to the compound (57 mg, 0.20 mmol) obtained in step 3) of Example 41 and 2-naphthylbenzaldehyde (35 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (75 mg, 89%).

¹H-NMR (300 MHz, CDCl₃) δ 8.52 (s, 1H), 8.16 (d, J=8.6 Hz, 1H), 7.73-7.79 (m, 3H), 7.45-7.60 (m, 2H), 7.20-7.40 (m, 5H), 6.87 (s, 1H), 4.20-4.35 (m, 2H), 3.77 (s, 2.7H), 3.54 (s, 0.3H), 2.99-3.12 (m, 2H).

MS (ESI): m/z=462.0635 [M+Na]⁺.

### Example 51

### Preparation of 1-methyl-5-((1-methyl-1H-imidazol-5-yl)methylene)-3-phenethyl-2-selenoxoimidazolidin-4-one (1ahq, Compound 51)

Dioxane (0.6 mL) was added to the compound (57 mg, 0.20 mmol) obtained in step 3) of Example 41 and 1-methyl-1H-imidazole-5-carboxaldehyde (24 mg, 0.21 mmol) and followed by stirring, piperidine (30 μl) and aluminum chloride (3 mg) were added, and the mixture was warmed at 90° C. for 5 h. After the reaction, the mixture was subjected to column separation to obtain the target compound (30 mg, 40%).

¹H-NMR (300 MHz, CD₃CN) δ 8.11 (dd, J=4.2, 1.2 Hz, 0.8H), 7.20-7.40 (m, 5H), 7.14 (s, 0.8H), 7.12 (s, 0.2H), 7.10-6.95 (m, 1.2H), 4.17-4.32 (m, 2H), 3.77 (s, 2H), 3.76 (s, 3H), 3.71 (s, 1H), 2.98-3.10 (m, 2H).

MS (ESI): m/z=373.0692 [M]⁺.

### Example 52

### Preparation of 1-methyl-5-(4-morpholinobenzylidene)-3-phenethyl-2-selenoxoimidazolidin-4-one (1ahs, Compound 52)

Dioxane (0.6 mL) was added to the compound (57 mg, 0.20 mmol) obtained in step 3) of Example 41 and 4-morpholinobenzaldehyde (40 mg, 0.21 mmol) and followed by stirring, piperidine (30 μl) and aluminum chloride (3 mg) were added, and the mixture was warmed at 90° C. for 5 h. After the reaction, the mixture was subjected to column separation to obtain the target compound (54 mg, 59%).

¹H-NMR (300 MHz, CD₃CN) δ 8.12 (d, J=9.2 Hz, 1.2H), 7.16-7.42 (m, 5.8H), 6.80-6.99 (m, 3H), 4.08-4.21 (m, 2H), 3.72-3.81 (m, 4H), 3.67 (s, 2H), 3.46 (s, 1H), 3.15-3.29 (m, 4H), 2.95-3.05 (m, 2H).

MS (ESI): m/z=455.1115 [M]⁺.

### Example 53

### Preparation of 5-(4-(4-(2-hydroxyethyl)piperazin-1-yl)benzylidene)-1-methyl-3-phenethyl-2-selenoxoimidazolidin-4-one (1ahu, Compound 53)

Dioxane (0.6 mL) was added to the compound (57 mg, 0.20 mmol) obtained in step 3) of Example 41 and 4-(4-(2-hydroxyethyl)piperazin-1-yl)benzaldehyde (49 mg, 0.21 mmol) and followed by stirring, piperidine (30 μl) and aluminum chloride (3 mg) were added, and the mixture was warmed at 90° C. for 5 h. After the reaction, the mixture was subjected to column separation to obtain the target compound (40 mg, 40%).

¹H-NMR (300 MHz, CD₃CN) δ 8.12 (d, J=9.0 Hz, 1H), 7.36 (d, J=8.9 Hz, 1H), 7.28 (s, 3H), 7.18-7.32 (m, 2H), 6.84-7.00 (m, 3H), 4.10-4.22 (m, 2H), 3.68 (s, 1.6H), 3.59 (t, J=5.6 Hz, 2H), 3.47 (s, 1.4H), 3.25-3.40 (m, 4H), 2.95-3.05 (m, 2H), 2.55-2.70 (m, 4H), 2.47-2.54 (m, 2H).

MS (EI): m/z=498.1532 [M]⁺.

### Example 54

### Preparation of 1-methyl-5-(4-((2-(morpholinoethyl)amino)benzylidene)-3-phenethyl-2-selenoxoimidazolidin-4-one (1ahw, Compound 54)

Dioxane (0.6 mL) was added to the compound (57 mg, 0.20 mmol) obtained in step 3) of Example 41 and 4-((2-morpholinoethyl)amino)benzaldehyde (49 mg, 0.21 mmol) and followed by stirring, piperidine (30 μl) and aluminum chloride (3 mg) were added, and the mixture was warmed at 90° C. for 5 h. After the reaction, the mixture was subjected to column separation to obtain the target compound (40 mg, 40%).

¹H-NMR (300 MHz, CD3CN) δ 8.11 (d, J=8.9 Hz, 1H), 7.88 (d, J=8.8 Hz, 1H), 7.10-7.40 (m, 5H), 6.88 (d, J=9.2 Hz, 1H), 6.55-6.72 (m, 2H), 5.26 (s, 1H), 4.16 (q, J=7.8 Hz, 2H), 3.70-3.80 (m, 1H), 3.68 (s, 1H), 3.65 (t, J=4.6 Hz, 4H), 3.51 (s, 2H), 3.15-3.30 (m, 2H), 2.85-3.08 (m, 3H), 2.59 (t, J=6.2 Hz, 3H), 2.46 (br s, 2H).

MS (EI): m/z=498.1537 [M]⁺.

### Example 55

### Preparation of 3-cyclohexyl-5-(2,4-dihydroxybenzylidene)-1-methyl-2-selenoxoimidazolidin-4-one (1aia, Compound 55)

### 1) Synthesis of N-cyclohexylformamide

After cyclohexylamine (21 g, 215 mmol) was added to formic acid (30 g, 644 mmol), the reaction was conducted under reflux at 100° C. for 4 hours and terminated. The reaction mixture was concentrated under reduced pressure and dried in a high vacuum to obtain the target compound (11 g, 40%).

¹H-NMR (300 MHz, CDCl₃) δ 8.11 (d, J=12.2 Hz, 1H), 6.31 (br s, 1H), 3.75-3.95 (m, 0.8H), 3.20-3.40 (m, 0.2H), 1.00-2.30 (m, 10H).

MS (EI): m/z=127 [M+H]⁺.

### 2) Synthesis of cyclohexyl isoselenocyanate

After N-(cyclohexyl)formylformamide (4.20 g, 33 mmol) was dissolved in methylene chloride (145 mL), triethylamine (20 mL, 4.30 mmol) and 4A MS (660 mg) were added. After triphosgene (5.3 g, 18 mmol) was dissolved in methylene chloride (25 mL), the solution was slowly added dropwise over 1 hour. The reaction mixture was refluxed for 4 h, then selenium (5.20 g, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (1.5 g, 24%).

¹H-NMR (300 MHz, CDCl₃) δ 3.50-3.65 (m, 1H), 1.55-1.93 (m, 6H), 1.20-1.53 (m, 4H).

MS (EI): m/z=189 [M+H]⁺.

### 3) Synthesis of 3-cyclohexyl-1-methyl-2-selenoxoimidazolidin-4-one

Dioxane (170 mL) and sarcosine (346 mg, 3.89 mmol) were added to the compound (732 mg, 3.89 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (645 mg, 64%).

¹H-NMR (300 MHz, CDCl₃) δ 4.60-4.82 (m, 1H), 3.80 (s, 2H), 3.40 (s, 3H), 2.10-2.40 (m, 2H), 1.55-1.90 (m, 5H), 1.10-1.50 (m, 3H).

MS (EI): m/z=260 [M+H]⁺.

### 4) Synthesis of 3-cyclohexyl-5-(2,4-dihydroxybenzylidene)-1-methyl-2-selenoxoimidazolidin-4-one (1aia)

Dioxane (0.6 mL) was added to the compound (52 mg, 0.2 mmol) obtained in step 3) and 2,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (33 mg, 43%).

¹H-NMR (300 MHz, DMSO-d₆) δ 10.3 (s, 1H), 10.1 (s, 1H), 8.52 (d, J=8.8 Hz, 1H), 7.23 (s, 1H), 6.37 (d, J=2.4 Hz, 1H), 6.25-6.32 (m, 1H), 4.65-4.87 (m, 1H), 3.66 (s, 3H), 2.12-2.30 (m, 2H), 1.55-1.88 (m, 5H), 1.10-1.38 (m, 3H).

MS (ESI): m/z=381.0715 [M+H]⁺.

### Example 56

### Preparation of 3-cyclohexyl-5-(2,3-dihydroxybenzylidene)-1-methyl-2-selenoxoimidazolidin-4-one (1aib, Compound 56)

Dioxane (0.6 mL) was added to the compound (52 mg, 0.20 mmol) obtained in step 3) of Example 55 and 3,5-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (46 mg, 61%).

¹H-NMR (300 MHz, DMSO-d₆) δ 9.57 (s, 1H), 9.23 (s, 1H), 7.74 (d, J=8.0 Hz, 1H), 7.22 (s, 1H), 6.86 (d, J=7.7 Hz, 1H), 6.63 (t, J=7.9 Hz, 1H), 4.60-4.90 (m, 1H), 3.67 (s, 3H), 2.05-2.40 (m, 2H), 1.52-1.90 (m, 5H), 1.02-1.45 (m, 3H).

MS (ESI): m/z=381.0715 [M+H]⁺.

### Example 57

### Preparation of 3-cyclohexyl-5-(4-(dimethylamino)benzylidene)-1-methyl-2-selenoxoimidazolidin-4-one (1aic, Compound 57)

Dioxane (0.6 mL) was added to the compound (52 mg, 0.20 mmol) obtained in step 3) of Example 55 and 4-dimethylaminobenzaldehyde (33 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (50 mg, 65%).

¹H-NMR (300 MHz, DMSO-d₆) δ 8.22 (d, J=8.4 Hz, 1.4H), 7.42 (d, J=8.1 Hz, 0.6H), 7.06 (s, 0.7H), 6.88 (s, 0.3H), 6.74 (d, J=8.4 Hz, 2H), 4.60-4.90 (m, 1H), 3.68 (s, 2H), 3.49 (s, 1H), 3.02 (s, 3H), 2.98 (s, 3H), 1.90-2.33 (m, 4H), 1.00-1.89 (m, 6H).

MS (ESI): m/z=392.1238 [M+H]⁺.

### Example 58

### Preparation of 3-cyclohexyl-5-(3,5-dihydroxybenzylidene)-1-methyl-2-selenoxoimidazolidin-4-one (1aie, Compound 58)

Dioxane (0.6 mL) was added to the compound (52 mg, 0.20 mmol) obtained in step 3) of Example 55 and 3,5-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (65 mg, 86%).

¹H-NMR (300 MHz, DMSO-d₆) δ 9.51 (s, 0.5H), 9.42 (s, 1.5H), 6.90-7.15 (m, 2H), 6.79 (s, 0.4H), 6.30 (d, J=4.3 Hz, 1H), 6.26 (s, 0.6H), 4.55-4.90 (m, 1H), 3.67 (s, 2H), 3.37 (s, 1H), 2.00-2.30 (m, 2H), 1.55-1.90 (m, 5H), 1.05-1.40 (m, 3H).

MS (ESI): m/z=381.0717 [M+H]⁺.

### Example 59

### Preparation of 5-(2,4-dihydroxybenzylidene)-3-hexyl-1-methyl-2-selenoxoimidazolidin-4-one (1aja, Compound 59)

### 1) Synthesis of N-hexylformamide

After n-hexylamine (22 g, 215 mmol) was added to formic acid (30 g, 644 mmol), the reaction was conducted under reflux at 100° C. for 4 hours and terminated. The reaction mixture was concentrated under reduced pressure and dried in a high vacuum to obtain the target compound (25 g, 91%).

¹H-NMR (300 MHz, CDCl₃) δ 8.16 (s, 0.8H), 8.04 (d, J=12.0 Hz, 0.2H), 5.65 (br s, 1H), 3.15-3.38 (m, 2H), 1.82 (br s, 2H), 1.20-1.44 (m, 5H), 0.80-0.95 (m, 3H).

MS (EI): m/z=129 [M]⁺.

### 2) Synthesis of N-hexyl isoselenocyanate

After N-hexylformylformamide (4.26 g, 33 mmol) was dissolved in methylene chloride (145 mL), triethylamine (20 mL, 4.30 mmol) and 4A MS (660 mg) were added. After triphosgene (5.3 g, 18 mmol) was dissolved in methylene chloride (25 mL), the solution was slowly added dropwise over 1 hour. The reaction mixture was refluxed for 4 h, then selenium (5.20 g, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (6.2 g, 99%).

¹H-NMR (300 MHz, CDCl₃) δ 3.61 (t, J=6.6 Hz, 0.2H), 1.67-1.82 (m, 2H), 1.26-1.52 (m, 6H), 0.85-0.98 (m, 3H).

MS (ESI): m/z=191 [M+H]⁺.

### 3) Synthesis of 3-hexyl-1-methyl-2-selenoxoimidazolidin-4-one

Dioxane (170 mL) and sarcosine (346 mg, 3.89 mmol) were added to the compound (740 mg, 3.89 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (945 mg, 93%).

¹H-NMR (300 MHz, CDCl₃) δ 3.88 (t, J=7.6 Hz, 0.2H), 3.86 (s, 2H), 3.42 (s, 3H), 1.53-1.80 (m, 2H), 1.20-1.40 (m, 5H), 0.80-0.95 (m, 3H).

MS (EI): m/z=262 [M+H]⁺.

### 4) Synthesis of 5-(2,4-dihydroxybenzylidene)-3-hexyl-1-methyl-2-selenoxoimidazolidin-4-one (1aja)

Dioxane (0.6 mL) was added to the compound (52 mg, 0.2 mmol) obtained in step 3) and 2,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (26 mg, 34%).

¹H-NMR (300 MHz, DMSO-d₆) δ 10.4 (s, 1H), 10.1 (s, 1H), 8.56 (d, J=8.8 Hz, 1H), 7.24 (s, 1H), 6.37 (s, 1H), 6.28 (d, J=8.9 Hz, 1H), 3.83-3.95 (m, 2H), 3.65 (s, 3H), 1.62 (br s, 2H), 1.26 (br s, 5H), 0.85 (br s, 3H).

MS (ESI): m/z=405.0690 [M+Na]⁺.

### Example 60

### Preparation of 5-(2,3-dihydroxybenzylidene)-3-hexyl-1-methyl-2-selenoxoimidazolidin-4-one (1ajb, Compound 60)

Dioxane (0.6 mL) was added to the compound (52 mg, 0.20 mmol) obtained in step 3) of Example 59 and 2,3-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (39 mg, 51%).

¹H-NMR (300 MHz, DMSO-d₆) δ 9.63 (br s, 1H), 9.30 (br s, 1H), 7.79 (d, J=8.0 Hz, 1H), 7.24 (s, 1H), 6.50-6.95 (m, 2H) 3.80-4.00 (m, 2H), 3.66 (s, 3H), 1.50-1.80 (br s, 2H), 1.26 (br s, 6H), 0.86 (br s, 3H).

MS (ESI): m/z=405.0689 [M+Na]⁺.

### Example 61

### Preparation of 5-(4-(dimethylamino)benzylidene)-3-hexyl-1-methyl-2-selenoxoimidazolidin-4-one (1ajc, Compound 61)

Dioxane (0.6 mL) was added to the compound (52 mg, 0.20 mmol) obtained in step 3) of Example 59 and 4-dimethylaminobenzaldehyde (33 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (46 mg, 59%).

¹H-NMR (300 MHz, DMSO-d₆) δ 8.24 (d, J=8.3 Hz, 1H), 7.07 (s, 1H), 6.75 (d, J=8.3 Hz, 1H), 3.82-3.95 (m, 2H), 3.67 (s, 3H), 3.02 (s, 6H), 1.63 (br s, 2H), 1.27 (br s, 6H), 0.85 (br s, 3H).

MS (ESI): m/z=394.1395 [M+H]⁺.

### Example 62

### Preparation of 5-(3,5-dihydroxybenzylidene)-3-hexyl-1-methyl-2-selenoxoimidazolidin-4-one (1aje, Compound 62)

Dioxane (0.6 mL) was added to the compound (52 mg, 0.20 mmol) obtained in step 3) of Example 59 and 3,5-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (51 mg, 67%).

¹H-NMR (300 MHz, DMSO-d₆) δ 9.55 (s, 0.5H), 9.45 (s, 1.5H), 7.08 (d, J=1.4 Hz, 1H), 6.95 (s, 0.7H), 6.86 (s, 0.3H), 6.25-6.40 (m, 1H), 3.80-3.95 (m, 2H), 3.66 (s, 3H), 1.62 (br s, 2H), 1.27 (br s, 6H), 0.85 (br s, 3H).

MS (ESI): m/z=405.0689 [M+Na]⁺.

### Example 63

### Preparation of 3-hexyl-1-methyl-5-(4-morpholinobenzylidene)-2-selenoxoimidazolidin-4-one (1ajs, Compound 63)

Dioxane (0.6 mL) was added to the compound (52 mg, 0.20 mmol) obtained in step 3) of Example 59 and 4-morpholinobenzaldehyde (49 mg, 0.21 mmol) and followed by stirring, piperidine (30 μl) and aluminum chloride (3 mg) were added, and the mixture was warmed at 90° C. for 5 h. After the reaction, the mixture was subjected to column separation to obtain the target compound (50 mg, 58%).

¹H-NMR (300 MHz, DMSO-d₆) δ 8.18 (d, J=9.1 Hz, 2H), 6.94 (d, J=9.1 Hz, 2H), 6.87 (s, 1H), 3.90-3.98 (m, 2H), 3.72-3.82 (m, 4H), 3.66 (s, 3H), 3.20-3.30 (m, 4H), 1.59-1.75 (m, 2H), 1.22-1.44 (m, 6H), 0.80-0.94 (m, 3H).

MS (EI): m/z=435.1425 [M]⁺.

### Example 64

### Preparation of 3-hexyl-5-(4-((2-(2-hydroxyethoxy)ethyl)amino)benzylidene)-1-methyl-2-selenoxoimidazolidin-4-one (1ajv, Compound 64)

Dioxane (0.5 mL) was added to the compound (23 mg, 0.09 mmol) obtained in step 3) of Example 59 and 4-((2-(2-hydroxy)ethyl)amino)benzaldehyde (18 mg, 0.22 mmol) and followed by stirring, piperidine (13 μl) and aluminum chloride (1 mg) were added, and the mixture was warmed at 90° C. for 5 h. After the reaction, the mixture was subjected to column separation to obtain the target compound (14 mg, 35%).

¹H-NMR (300 MHz, DMSO-d₆) δ 8.15 (d, J=8.9 Hz, 1.4H), 7.29 (d, J=8.8 Hz, 0.6H), 6.92 (s, 0.3H), 6.85 (s, 0.7H), 6.67 (d, J=8.9 Hz, 2H), 5.39 (br s, 0.7H), 5.18 (br s, 0.3H), 3.89-3.99 (m, 2H), 3.66 (s, 2.1H), 3.57-3.65 (m, 4H), 3.49-3.55 (m, 2H), 3.48 (s, 0.9H), 3.26-3.37 (m, 2H), 2.89-2.97 (m, 1H), 1.22-1.78 (m, 8H), 0.80-0.95 (m, 3H).

MS (EI): m/z=453.1534 [M]⁺.

### Example 65

### Preparation of 3-hexyl-1-methyl-5-(4-((2-morpholinoethyl)amino)benzylidene)-2-selenoxoimidazolidin-4-one (1ajw, Compound 65)

Dioxane (0.8 mL) was added to the compound (40 mg, 0.15 mmol) obtained in step 3) of Example 59 and 4-(2-morpholinoethyl)aminobenzaldehyde (36 mg, 0.15 mmol) and followed by stirring, piperidine (23 μl) and aluminum chloride (2 mg) were added, and the mixture was warmed at 90° C. for 5 h. After the reaction, the mixture was subjected to column separation to obtain the target compound (25 mg, 34%).

¹H-NMR (300 MHz, CD3CN) δ 8.15 (d, J=8.9 Hz, 0.6H), 7.29 (d, J=8.5 Hz, 0.4H), 6.91 (s, 0.4H), 6.85 (s, 0.6H), 6.65 (d, J=8.8 Hz, 1H), 5.35 (br s, 0.5H), 3.94 (q, J=7.4 Hz, 2H), 3.66 (s, 2H), 3.64 (t, J=4.6 Hz, 2H), 3.49 (s, 1H), 3.12-3.29 (m, 2H), 2.57 (t, J=6.2 Hz, 2H), 2.40-2.50 (m, 4H), 1.62-1.76 (m, 2H), 1.46-1.61 (m, 2H), 1.20-1.40 (m, 6H), 0.83-0.95 (m, 3H).

MS (EI): m/z=478.1849 [M]⁺.

### Example 66

### Preparation of 5-(4-fluorobenzylidene)-3-hexyl-1-methyl-2-selenoxoimidazolidin-4-one (1ajx, Compound 66)

Dioxane (1.2 mL) was added to the compound (105 mg, 0.4 mmol) obtained in step 3) of Example 59 and 4-fluorobenzaldehyde (52 mg, 0.42 mmol) and followed by stirring, piperidine (60 μl) and aluminum chloride (6 mg) were added, and the mixture was warmed at 90° C. for 5 h. After the reaction, the mixture was subjected to column separation to obtain the target compound (18 mg, 12%).

¹H-NMR (300 MHz, CD₃CN) δ 8.13 (d, J=8.9 Hz, 1H), 6.84 (s, 1H), 7.37 (d, J=8.5 Hz, 1H), 7.17-7.32 (m, 1H), 6.83-6.98 (m, 2H), 4.08-4.20 (m, 1H), 3.77 (t, J=4.8 Hz, 2H), 3.68 (s, 1.7H), 3.47 (s, 1.3H), 3.17-3.30 (m, 2H), 2.95-3.06 (m, 1H), 1.15-1.36 (m, 4H), 0.75-0.96 (m, 3H).

MS (EI): m/z=368.0806 [M+]⁺.

### Example 67

### Preparation of 5-(4-((2-(2-fluoroethoxy)ethyl)amino)benzylidene)-3-hexyl-1-methyl-2-selenoxoimidazolidin-4-one (1ajy, Compound 67)

Dioxane (4.7 mL) was added to the compound (654 mg, 2.49 mmol) obtained in step 3) of Example 59 and 4-((2-(2-fluoroethoxy)ethyl)amino)benzaldehyde (527 mg, 2.49 mmol) and followed by stirring, piperidine (370 μl) and aluminum chloride (66 mg) were added, and the mixture was warmed at 90° C. for 5 h. After the reaction, the mixture was subjected to column separation to obtain the target compound (791 mg, 70%).

¹H-NMR (300 MHz, CD₃CN) δ 8.15 (d, J=8.9 Hz, 2H), 6.84 (s, 1H), 6.66 (d, J=8.9 Hz, 2H), 5.28 (br s, 1H), 4.62 (t, J=4.0 Hz, 1H), 4.46 (t, J=4.0 Hz, 1H), 3.95 (t, J=7.5 Hz, 2H), 3.75 (t, J=4.0 Hz, 1H) 3.65 (s, 3H), 3.60-3.72 (m, 3H), 1.60-1.75 (m, 2H), 1.24-1.38 (m, 6H), 0.82-0.94 (m, 3H).

MS (EI): m/z=455.1484 [M+]⁺.

### Example 68

### Preparation of 3-cyclopentyl-5-(2,4-dihydroxybenzylidene)-1-methyl-2-selenoxoimidazolidin-4-one (1aka, Compound 68)

### 1) Synthesis of N-cyclopentylformamide

After cyclopentylamine (27 g, 322 mmol) was added to formic acid (30 g, 644 mmol), the reaction was conducted under reflux at 90° C. for 48 hours and terminated. The reaction mixture was concentrated under reduced pressure and dried in a high vacuum to obtain the target compound (26 g, 71%).

¹H-NMR (300 MHz, CDCl₃) δ 8.13 (s, 0.2H), 8.09 (s, 0.8H),), 5.98 (br s, 1H), 4.20-4.38 (m, 0.8H), 3.78-3.95 (m, 0.2H), 1.95-2.09 (m, 2H), 1.55-1.79 (m, 4H), 1.35-1.54 (m, 2H).

MS (EI): m/z=113 [M]⁺.

### 2) Synthesis of N-cyclopentyl isoselenocyanate

After N-(cyclohexyl)formamide (4.6 g, 33 mmol) was dissolved in methylene chloride (145 mL), triethylamine (20 mL, 4.30 mmol) and 4A MS (660 mg) were added. After triphosgene (5.3 g, 18 mmol) was dissolved in methylene chloride (25 mL), the solution was slowly added dropwise over 1 hour. The reaction mixture was refluxed for 4 h, then selenium (5.20 g, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (3.4 g, 51%).

¹H-NMR (300 MHz, CDCl3) δ 7.20-7.35 (m, 2H), 7.00 (7.13 (m, 2H).

MS (EI): m/z=175 [M]+.

### 3) Synthesis of 3-cyclopentyl-1-methyl-2-selenoxoimidazolidin-4-one

Dioxane (170 mL) and sarcosine (346 mg, 3.89 mmol) were added to the compound (778 mg, 3.89 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (382 mg, 40%).

¹H-NMR (300 MHz, CDCl₃) δ 5.15 (quintet, J=8.5 Hz, 1H), 3.81 (s, 2H), 3.42 (s, 3H), 2.00-2.20 (m, 2H), 1.83-1.99 (m, 4H), 1.53-1.70 (m, 2H).

MS (EI): m/z=246 [M+H]⁺.

### 4) Synthesis of 3-cyclopentyl-5-(2,4-dihydroxybenzylidene)-1-methyl-2-selenoxoimidazolidin-4-one (1aka)

Dioxane (0.6 mL) was added to the compound (52 mg, 0.2 mmol) obtained in step 3) and 2,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (63 mg, 81%).

¹H-NMR (300 MHz, DMSO-d₆) δ 10.4 (s, 1H), 10.1 (s, 1H), 8.53 (d, J=8.8 Hz, 1H), 7.24 (s, 1H), 6.37 (d, J=8.8 Hz, 1H), 5.21-5.31 (m, 1H), 1.98-2.15 (m, 2H), 1.68-1.88 (m, 4H), 1.48-1.67 (m, 2H).

MS (ESI): m/z=389.0377 [M+Na]⁺.

### Example 69

### Preparation of 3-cyclopentyl-5-(2,3-dihydroxybenzylidene)-1-methyl-2-selenoxoimidazolidin-4-one (1akb, Compound 69)

Dioxane (0.3 mL) was added to the compound (25 mg, 0.1 mmol) obtained in step 3) of Example 68 and 2,3-dihydroxybenzaldehyde (15 mg, 0.11 mmol) and followed by stirring, piperidine (15 μl) and aluminum chloride (1.3 mg) were added, and the mixture was warmed at 90° C. for 5 h. After the reaction, the mixture was subjected to column separation to obtain the target compound (18 mg, 49%).

¹H-NMR (300 MHz, DMSO-d₆) δ 7.04 (dt, J=7.7, 1.7 Hz, 1H), 6.85-7.00 (m, 2H), 6.69-6.86 (m, 1H), 3.79 (s, 1H), 3.44 (s, 2H), 2.05-2.30 (m, 2H), 1.85-2.05 (m, 4H), 1.53-1.78 (m, 2H).

MS (ESI): m/z=389.0379 [M+Na]⁺.

### Example 70

### Preparation of 3-cyclopentyl-5-(4-(dimethylamino)benzylidene)-1-methyl-2-selenoxoimidazolidin-4-one (1akc, Compound 70)

Dioxane (0.3 mL) was added to the compound (25 mg, 0.1 mmol) obtained in step 3) of Example 68 and 4-dimethylaminobenzaldehyde (16 mg, 0.11 mmol) and followed by stirring, piperidine (15 μl) and aluminum chloride (1.3 mg) were added, and the mixture was warmed at 90° C. for 5 h. After the reaction, the mixture was subjected to column separation to obtain the target compound (36 mg, 96%).

¹H-NMR (300 MHz, CDCl₃) δ 8.21 (d, J=9.0 Hz, 2H), 7.07 (s, 1H), 6.75 (d, J=9.0 Hz, 2H), 5.10-5.40 (m, 1H), 3.69 (s, 3H), 3.02 (s, 6H), 1.96-2.18 (m, 2H), 1.75-1.93 (m, 4H), 1.52-1.69 (m, 2H).

MS (ESI): m/z=378.1079 [M+H]⁺.

### Example 71

### Preparation of 3-cyclopentyl-5-(3,4-dihydroxybenzylidene)-1-methyl-2-selenoxoimidazolidin-4-one (1akd, Compound 71)

Dioxane (0.3 mL) was added to the compound (25 mg, 0.1 mmol) obtained in step 3) of Example 68 and 3,4-dihydroxybenzaldehyde (15 mg, 0.11 mmol) and followed by stirring, piperidine (15 μl) and aluminum chloride (1.3 mg) were added, and the mixture was warmed at 90° C. for 5 h. After the reaction, the mixture was subjected to column separation to obtain the target compound (27 mg, 73%).

¹H-NMR (300 MHz, DMSO-d₆) δ 9.80 (br s, 1H), 9.26 (br s, 1H), 7.94 (d, J=1.9 Hz, 1H), 7.53 (dd, J=8.4, 1.9 Hz, 1H), 7.01 (s, 1H), 5.28 (quintet, J=8.6 Hz, 1H), 3.68 (s, 3H), 2.00-2.20 (m, 2H), 1.73-1.95 (m, 4H), 1.50-1.67 (m, 2H).

MS (ESI): m/z=367.0561 [M+H]⁺.

### Example 72

### Preparation of 3-cyclopentyl-5-(3,5-dihydroxybenzylidene)-1-methyl-2-selenoxoimidazolidin-4-one (1ake, Compound 72)

Dioxane (0.3 mL) was added to the compound (25 mg, 0.1 mmol) obtained in step 3) of Example 68 and 3,5-dihydroxybenzaldehyde (15 mg, 0.11 mmol) and followed by stirring, piperidine (15 μl) and aluminum chloride (1.3 mg) were added, and the mixture was warmed at 90° C. for 5 h. After the reaction, the mixture was subjected to column separation to obtain the target compound (36 mg, 100%).

¹H-NMR (300 MHz, DMSO-d₆) δ 9.52, 9.42 (each s (1:3), 2H), 7.04 (d, J=2.0 Hz, 1H), 6.95 (s, 1H), 6.80 (s, 0.4H), 5.10-5.37 (m, 1H), 3.67 (s, 2.2H), 3.38 (s, 0.8H), 1.96-2.16 (m, 2H), 1.73-1.95 (m, 4H), 1.48-1.67 (m, 2H).

MS (ESI): m/z=369.0376 [M+Na]⁺.

### Example 73

### Preparation of 5-(2,4-dihydroxybenzylidene)-3-(4-fluorophenyl)-1-methyl-2-selenoxoimidazolidin-4-one (1ala, Compound 73)

1) Synthesis of N-(4-fluorophenyl)formamide

After 4-fluorophenylamine (24 g, 215 mmol) was added to formic acid (30 g, 644 mmol), the reaction was conducted under reflux at 100° C. for 4 hours and terminated. After ethyl acetate was added to the reaction mixture for dilution, the organic layer was washed with water, 0.1 N aqueous hydrochloric acid solution, saturated aqueous sodium hydrogen carbonate solution, and brine. The organic layer was dried over anhydrous sodium sulfate, filtered, and concentrated under reduced pressure to obtain the target compound (24 g, 79%).

¹H-NMR (300 MHz, CDCl₃) δ 8.57 (d, J=11.4 Hz, 0.4H), 8.37 (s, 0.6H), 7.80 (br s, 0.4H), 7.45-7.56 (m, 1H), 7.23 (br s, 0.6H), 6.97-7.10 (m, 3H).

MS (EI): m/z=139 [M]⁺.

### 2) Synthesis of N-(4-fluorophenyl)isoselenocyanate

After N-(4-fluorophenyl)formamide (4.6 g, 33 mmol) was dissolved in methylene chloride (145 mL), triethylamine (20 mL, 4.30 mmol) and 4A MS (660 mg) were added. After triphosgene (5.3 g, 18 mmol) was dissolved in methylene chloride (25 mL), the solution was slowly added dropwise over 1 hour. The reaction mixture was refluxed for 4 h, then selenium (5.20 g, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (3.4 g, 51%).

¹H-NMR (300 MHz, CDCl3) δ 7.20-7.35 (m, 2H), 7.00 (7.13 (m, 2H).

MS (EI): m/z=201 [M]⁺.

### 3) Synthesis of 3-(4-fluorophenyl)-1-methyl-2-selenoxoimidazolidin-4-one

Dioxane (170 mL) and sarcosine (346 mg, 3.89 mmol) were added to the compound (778 mg, 3.89 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (939 mg, 89%).

¹H-NMR (300 MHz, CDCl₃) δ 7.10-7.40 (m, 4H), 4.08 (s, 2H), 3.51 (s, 3H).

MS (EI): m/z=272 [M+H]⁺.

### 4) Synthesis of 5-(2,4-dihydroxybenzylidene)-3-(4-fluorophenyl)-1-methyl-2-selenoxoimidazolidin-4-one (1ala)

Dioxane (0.6 mL) was added to the compound (52 mg, 0.2 mmol) obtained in step 3) and 2,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (63 mg, 81%).

¹H-NMR (300 MHz, DMSO-d₆) δ 10.4 (s, 1H), 10.1 (s, 1H), 8.50 (d, J=8.8 Hz, 1H), 7.28-7.60 (m, 5H), 6.38 (d, J=2.2 Hz, 1H), 6.26 (dd, J=8.8, 2.2 Hz, 1H), 3.75 (s, 3H).

MS (ESI): m/z=393.0150 [M+H]⁺.

### Example 74

### Preparation of 5-(2,3-dihydroxybenzylidene)-3-(4-fluorophenyl)-1-methyl-2-selenoxoimidazolidin-4-one (1alb, Compound 74)

Dioxane (1.5 mL) was added to the compound (136 mg, 0.5 mmol) obtained in step 3) of Example 73 and 2,3-dihydroxybenzaldehyde (78 mg, 0.6 mmol) and followed by stirring, piperidine (74 μl) and aluminum chloride (7 mg) were added, and the mixture was warmed at 90° C. for 5 h. After the reaction, the mixture was subjected to column separation to obtain the target compound (98 mg, 50%).

¹H-NMR (300 MHz, DMSO-d₆) δ 9.40 (br s, 2H), 7.28-7.53 (m, 4H), 7.00 (s, 1H), 6.60-6.90 (m, 2H), 3.76 (s, 1H), 3.46 (s, 2H).

MS (ESI): m/z=393.0149 [M+1]⁺.

### Example 75

### Preparation of 5-(4-(dimethylamino)benzylidene)-3-(4-fluorophenyl)-1-methyl-2-selenoxoimidazolidin-4-one (1alc, Compound 75)

Dioxane (1.5 mL) was added to the compound (136 mg, 0.5 mmol) obtained in step 3) of Example 73 and 4-dimethylaminobenzaldehyde (82 mg, 0.6 mmol) and followed by stirring, piperidine (74 μl) and aluminum chloride (7 mg) were added, and the mixture was warmed at 90° C. for 5 h. After the reaction, the mixture was subjected to column separation to obtain the target compound (199 mg, 90%).

¹H-NMR (300 MHz, DMSO-d₆) δ 8.19 (d, J=9.1 Hz, 2H), 7.32-7.44 (m, 2H), 7.13-7.24 (m, 2H), 6.80 (s, 1H), 6.67 (d, J=9.1 Hz, 1H), 3.85 (s, 3H), 3.06 (s, 6H).

MS (ESI): m/z=404.0674 [M+H]⁺.

### Example 76

### Preparation of 5-(3,4-dihydroxybenzylidene)-3-(4-fluorophenyl)-1-methyl-2-selenoxoimidazolidin-4-one (1ald, Compound 76)

Dioxane (1.5 mL) was added to the compound (136 mg, 0.5 mmol) obtained in step 3) of Example 73 and 3,4-dihydroxybenzaldehyde (78 mg, 0.6 mmol) and followed by stirring, piperidine (74 μl) and aluminum chloride (7 mg) were added, and the mixture was warmed at 90° C. for 5 h. After the reaction, the mixture was subjected to column separation to obtain the target compound (67 mg, 34%).

¹H-NMR (300 MHz, DMSO-d₆) δ 9.56 (s, 2H), 7.29-7.57 (m, 5H), 6.93 (s, 1H), 6.35 (d, J=2.0 Hz, 1H), 6.20-6.32 (m, 1H), 3.46 (s, 3H).

MS (ESI): m/z=414.9971 [M+1]⁺.

### Example 77

### Preparation of 5-(3,5-dihydroxybenzylidene)-3-(4-fluorophenyl)-1-methyl-2-selenoxoimidazolidin-4-one (1ale, Compound 77)

Dioxane (3.0 mL) was added to the compound (272 mg, 1.0 mmol) obtained in step 3) of Example 73 and 3,5-dihydroxybenzaldehyde (155 mg, 1.0 mmol) and followed by stirring, piperidine (148 μl) and aluminum chloride (13 mg) were added, and the mixture was warmed at 90° C. for 5 h. After the reaction, the mixture was subjected to column separation to obtain the target compound (153 mg, 39%).

¹H-NMR (300 MHz, DMSO-d₆) δ 9.56 (s, 0.5H), 9.41 (s, 1.5H), 7.40-7.56 (m, 2H), 7.30-7.40 (m, 2H), 7.08 (d, J=2.2 Hz, 1H), 7.05 (s, 1H), 6.20-6.52 (m, 2H), 3.76 (s, 2H), 3.46 (s, 1H).

MS (ESI): m/z=393.0149 [M+H]⁺.

### Example 78

### Preparation of 5-(2,4-dihydroxybenzylidene)-1-methyl-3-octyl-2-selenoxoimidazolidin-4-one (1ama, Compound 78)

### 1) Synthesis of N-octylformamide

After N-octylamine (6.9 mL 32 mmol) was added to formic acid (3.6 mL, 95 mmol), the reaction was conducted under reflux at 90° C. for 48 hours and terminated. The reaction mixture was concentrated under reduced pressure, dried in a high vacuum, and subjected to column separation to obtain the target compound (5.5 g, 94%).

H-NMR (300 MHz, CDCl₃) δ 8.15 (s, 1H), 6.17 (br s, 1H), 3.17-3.31 (m, 2H), 1.48-1.55 (m, 2H), 1.24-1.28 (m, 10H), 0.88 (t, J=6.6 Hz, 3H).

MS (EI): m/z=156 [M-1]⁺.

### 2) Synthesis of N-octyl isoselenocyanate

After N-octylformamide (1.0 g, 5.8 mmol) was dissolved in EDC (20 mL), triethylamine (3.5 mL, 25 mmol) and 4A MS (1 g) were added. After triphosgene (930 mg, 3.1 mmol) was dissolved in EDC (10 mL), the solution was slowly added dropwise over 1 hour. The reaction mixture was refluxed for 4 h, then selenium (917 mg, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (912 mg, 67%).

¹H-NMR (300 MHz, CDCl₃) δ 3.60 (t, J=6.6 Hz, 2H), 1.73 (q, J=7.7 Hz, 2H), 1.29-1.44 (m, 10H), 0.89 (t, J=7.0 Hz, 3H).

### 3) Synthesis of N-octyl-1-methyl-2-selenoxoimidazolidin-4-one

Dioxane (10 mL) and sarcosine (123 mg, 1.4 mmol) were added to the compound (300 mg, 1.37 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (320 mg, 81%).

¹H-NMR (300 MHz, CDCl₃) δ 3.86-3.91 (m, 4H), 3.42 (s, 3H), 1.63-1.73 (m, 2H), 1.27-1.32 (m, 10H), 0.89 (t, J=7.0 Hz, 3H).

### 4) Synthesis of 5-(2,4-dihydroxybenzylidene)-1-methyl-3-octyl-2-selenoxoimidazolidin-4-one (1ama)

Dioxane (2 mL) was added to the compound (150 mg, 0.52 mmol) obtained in step 3) and 2,4-dihydroxybenzaldehyde (86 mg, 0.62 mmol) and followed by stirring, piperidine (77 μl) and aluminum chloride (14 mg) were added, and the mixture was warmed at 90° C. for 5 h. After the reaction, the mixture was subjected to column separation to obtain the target compound (124 mg, 58%).

¹H-NMR (300 MHz, DMSO-d₆) δ 10.4 (s, 1H), 10.2 (s, 1H), 8.57 (d, J=8.8 Hz, 1H), 7.24 (s, 1H), 6.37 (d, J=0.2 Hz, 1H), 6.29 (dd, J=8.8, 2.6 Hz, 1H 1H), 3.88 (t J=7.3 Hz, 2H), 3.65 (s, 3H), 1.60-1.64 (m, 2H), 1.24-1.26 (m, 10H), 0.84 (t, J=6.4 Hz, 3H).

MS (EI): m/z=410.1110 [M]⁺.

### Example 79

### Preparation of 5-(3,5-dihydroxybenzylidene)-1-methyl-3-octyl-2-selenoxoimidazolidin-4-one (1ame, Compound 79)

Dioxane (1 mL) was added to the compound (70 mg, 0.24 mmol) obtained in step 3) of Example 78 and 3,5-dihydroxybenzaldehyde (40 mg, 0.29 mmol) and followed by stirring, piperidine (36 μl) and aluminum chloride (6.4 mg) were added, and the mixture was warmed at 90° C. for 5 h. After the reaction, the mixture was subjected to column separation to obtain the target compound (91 mg, 92%).

¹H-NMR (300 MHz, DMSO-d₆) δ 9.52 (s, 1H), 9.42 (s, 1H), 7.15 (s, 1H), 6.96 (s, 0.3H), 6.86 (s, 0.7H), 6.27-6.36 (m, 2H), 3.88 (t, J=7.3 Hz, 2H), 3.66 (s, 3H), 1.63 (s, 2H), 1.25-1.27 (m, 10H), 0.85 (t, J=6.6 Hz, 3H).

MS (EI): m/z=410.1110 [M]⁺.

### Example 80

### Preparation of 3-decyl-5-(2,4-dihydroxybenzylidene)-1-methyl-2-selenoxoimidazolidin-4-one (1ana, Compound 80)

### 1) Synthesis of N-decylformamide

After N-octylamine (6.9 mL 32 mmol) was added to formic acid (3.6 mL, 95 mmol), the reaction was conducted under reflux at 90° C. for 48 hours and terminated. The reaction mixture was concentrated under reduced pressure, dried in a high vacuum, and subjected to column separation to obtain the target compound (5.5 g, 94%).

¹H-NMR (300 MHz, CDCl₃) δ 8.16 (s, 0.2H), 5.74 (br s, 1H), 3.29 (q, J=6.7 Hz, 2H), 1.48-1.55 (m, 3H), 1.20-1.29 (m, 13H), 0.88 (t, J=6.8 Hz, 3H).

MS (EI): m/z=184 [M-1]⁺.

### 2) Synthesis of N-decyl isoselenocyanate

After N-decylformamide (500 mg, 2.7 mmol) was dissolved in EDC (24 mL), triethylamine (1.6 mL, 25 mmol) and 4A MS (0.1 g) were added. After triphosgene (432 mg, 3.1 mmol) was dissolved in EDC (16 mL), the solution was slowly added dropwise over 1 hour. The reaction mixture was refluxed for 4 h, then selenium (426 mg, 5.4 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (462 mg, 69%).

¹H-NMR (300 MHz, CDCl₃) δ 3.59 (t, J=6.7 Hz, 2H), 1.73 (quintet, J=6.7 Hz, 1H), 1.27-1.44 (m, 14H), 0.88 (t, J=6.7 Hz, 3H).

MS (EI): m/z=247 [M]⁺.

### 3) Synthesis of N-decyl-1-methyl-2-selenoxoimidazolidin-4-one

Dioxane (10 mL) and sarcosine (108 mg, 1.4 mmol) were added to the compound (300 mg, 1.37 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (289 mg, 75%).

¹H-NMR (300 MHz, CDCl₃) δ 3.86-3.91 (m, 4H), 3.42 (s, 2H), 1.27-1.44 (m, 3H), 1.63-1.73 (m, 2H), 1.26-1.32 (m, 14H), 0.88 (t, J=6.6 Hz, 3H).

### 4) Synthesis of 3-decyl-5-(2,4-dihydroxybenzylidene)-1-methyl-2-selenoxoimidazolidin-4-one (1ana)

Dioxane (1.5 mL) was added to the compound (100 mg, 0.32 mmol) obtained in step 3) and 2,4-dihydroxybenzaldehyde (52 mg, 0.38 mmol) and followed by stirring, piperidine (47 μl) and aluminum chloride (8.4 mg) were added, and the mixture was warmed at 90° C. for 5 h. After the reaction, the mixture was subjected to column separation to obtain the target compound (94 mg, 69%).

¹H-NMR (300 MHz, DMSO-d₆) δ 10.4 (s, 1H), 10.1 (s, 1H), 8.57 (d, J=8.8 Hz, 1H), 7.24 (s, 1H), 6.37 (d, J=2.3 Hz, 1H), 6.28 (dd, J=8.8, 2.2 Hz, 1H), 3.88 (t, J=7.3 Hz, 2H), 3.65 (s, 3H), 1.57-1.67 (m, 2H), 1.17-1.26 (m, 14H), 10.84 (t, J=7.0 Hz, 3H).

MS (EI): m/z=438.1425 [M]⁺.

### Example 81

### Preparation of 5-(2,4-dihydroxybenzylidene)-1-methyl-3-(2-morpholinoethyl)-2-selenoxoimidazolidin-4-one (1apa, Compound 81)

### 1) Synthesis of N-(3-morpholinoethyl)formamide

After N-(3-aminoethyl)morpholine (3.0 mL 21 mmol) was added to formic acid (2.6 mL, 69 mmol), the reaction was conducted under reflux at 90° C. for 48 hours and terminated. The reaction mixture was concentrated under reduced pressure, dried in a high vacuum, and subjected to column separation to obtain the target compound (4.6 g, 122%).

¹H-NMR (300 MHz, CDCl₃) δ 8.30 (s, 1), 7.11 (br s, 1H),), 3.83 (t, J=4.7 Hz, 4H), 3.56 (q, J=5.7 Hz, 2H), 2.79-2.83 (m, 6H).

MS (EI): m/z=158 [M]⁺.

### 2) Synthesis of N-(3-morpholinoethyl) isoselenocyanate

After N-(3-morpholinoethyl)formamide (1.0 g, 6.3 mmol) was dissolved in EDC (20 mL), triethylamine (3.7 mL, 27 mmol) and 4A MS (1 g) were added. After triphosgene (930 mg, 3.1 mmol) was dissolved in EDC (10 mL), the solution was slowly added dropwise over 1 hour. The reaction mixture was refluxed for 4 h, then selenium (998 mg, 13 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (452 mg, 33%).

¹H-NMR (300 MHz, CDCl3) δ 3.68-3.74 (m, 6H), 2.71 (t, J=6.2 Hz, 2H), 2.52 (t, J=4.7 Hz, 4H).

MS (EI): m/z=220 [M]⁺.

### 3) Synthesis of N-(3-morpholinoethyl)-1-methyl-2-selenoxoimidazolidin-4-one

Dioxane (12 mL) and sarcosine (184 mg, 2.06 mmol) were added to the compound (452 mg, 2.06 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (368 mg, 61%).

¹H-NMR (300 MHz, CDCl₃) δ 4.04 (t, J=6.6 Hz, 2H), 3.89 (s, 2H), 3.65-3.70 (m, 4H), 3.43 (s, 3H), 2.68 (t, J=6.6 Hz, 2H), 2.52-2.55 (m, 3H), 2.40-2.45 (m, 1H).

MS (EI): m/z=291 [M]⁺.

### 4) Production of 5-(2,4-dihydroxybenzylidene)-1-methyl-3-(2-morpholinoethyl)-2-selenoxoimidazolidin-4-one (1apa)

Dioxane (1.5 mL) was added to the compound (100 mg, 0.33 mmol) obtained in step 3) and 2,4-dihydroxybenzaldehyde (57 mg, 0.41 mmol) and followed by stirring, piperidine (51 μl) and aluminum chloride (9.2 mg) were added, and the mixture was warmed at 90° C. for 5 h. After the reaction, the mixture was subjected to column separation to obtain the target compound (42 mg, 30%).

¹H-NMR (300 MHz, DMSO-d₆) δ 10.4 (s, 1H), 10.2 (s, 1H), 8.56 (d, J=8.8 Hz, 1H), 7.25 (s, 1H), 6.38 (d, J=2.1 Hz, 1H), 6.29 (dd, J=8.9, 2.1 Hz, 1H), 4.03 (t, J=6.6 Hz, 2H), 3.66 (s, 3H), 3.53 (t, J=? Hz, 6H), 2.58 (t, J=6.7 Hz, 2H), 2.44 (s, 2H).

MS (EI): m/z=411.0698 [M]⁺.

### Example 82

### Preparation of 5-(3,5-dihydroxybenzylidene)-1-methyl-3-(2-morpholinoethyl)-2-selenoxoimidazolidin-4-one (1ape, Compound 82)

Dioxane (1.5 mL) was added to the compound (100 mg, 0.33 mmol) obtained in step 3) of Example 81 and 3,5-dihydroxybenzaldehyde (57 mg, 0.41 mmol) and followed by stirring, piperidine (51 μl) and aluminum chloride (9.2 mg) were added, and the mixture was warmed at 90° C. for 5 h. After the reaction, the mixture was subjected to column separation to obtain the target compound (4.9 mg, 3.5%).

¹H-NMR (300 MHz, DMSO-d₆) δ 9.53 (s, 1H), 9.44 (s, 1H), 7.08 (s, 1H), 7.24 (s, 1H), 6.97 (s, 0.4H), 6.88 (s, 0.6H), 6.30 (s, 1H), 6.22-6.38 (m, 1H), 3.96-4.08 (m, 2H), 3.67 (s, 1.3H), 3.47-3.58 (m, 4H), 3.38 (s, 1.7H), 2.54-2.65 (m, 2H), 2.35-2.47 (m, 4H).

MS (EI): m/z=411.0701 [M]⁺.

### Example 83

### Preparation of 5-(2,4-dihydroxybenzylidene)-1-methyl-3-(2-morpholinopropyl)-2-selenoxoimidazolidin-4-one (1aqa, Compound 83)

### 1) Synthesis of N-(3-morpholinopropyl)formamide

After N-(3-aminoethyl)morpholine (3.0 mL 21 mmol) was added to formic acid (2.4 mL, 62 mmol), the reaction was conducted under reflux at 90° C. for 48 hours and terminated. The reaction mixture was concentrated under reduced pressure, dried in a high vacuum, and subjected to column separation to obtain the target compound (4.4 g, 122%).

¹H-NMR (300 MHz, CDCl₃) δ 8.34 (s, 1H), 7.03 (br s, 1H),), 3.86 (t, J=4.7 Hz, 4H), 3.37 (q, J=6.4 Hz, 2H), 2.78-2.85 (m, 6H), 1.90 (quintet, J=6.1 Hz, 2H).

MS (EI): m/z=172 [M]⁺.

### 2) Synthesis of N-(3-morpholinopropyl) isoselenocyanate

After N-(3-morpholinopropyl)formamide (1.0 g, 5.8 mmol) was dissolved in EDC (20 mL), triethylamine (3.5 mL, 25 mmol) and 4A MS (1 g) were added. After triphosgene (930 mg, 3.1 mmol) was dissolved in EDC (10 mL), the solution was slowly added dropwise over 1 hour. The reaction mixture was refluxed for 4 h, then selenium (917 mg, 66 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (912 mg, 67%).

¹H-NMR (300 MHz, CDCl₃) δ 3.69-3.73 (m, 6H), 2.42-2.48 (m, 6H), 1.89 (quintet, J=6.6 Hz, 2H).

MS (EI): m/z=234 [M]⁺.

### 3) Synthesis of N-(3-morpholinoethyl)-1-methyl-2-selenoxoimidazolidin-4-one

Dioxane (12 mL) and sarcosine (344 mg, 3.86 mmol) were added to the compound (900 mg, 3.86 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (975 mg, 83%).

¹H-NMR (300 MHz, CDCl₃) δ 4.98 (t, J=7.3 Hz, 2H), 3.87 (s, 2H), 3.69 (t, J=4.6 Hz, 4H), 3.42 (s, 3H), 2.40-2.45 (m, 6H), 1.89 (quintet, J=7.3 Hz, 2H).

MS (EI): m/z=305 [M]⁺.

### 4) Synthesis of 5-(2,4-dihydroxybenzylidene)-1-methyl-3-(2-morpholinopropyl)-2-selenoxoimidazolidin-4-one (1aqa)

Dioxane (1.5 mL) was added to the compound (100 mg, 0.33 mmol) obtained in step 3) and 2,4-dihydroxybenzaldehyde (55 mg, 0.39 mmol) and followed by stirring, piperidine (49 μl) and aluminum chloride (8.8 mg) were added, and the mixture was warmed at 90° C. for 5 h. After the reaction, the mixture was subjected to column separation to obtain the target compound (73 mg, 53%).

¹H-NMR (300 MHz, DMSO-d₆) δ 8.58 (d, J=8.8 Hz, 1H), 7.24 (s, 1H), 6.37 (d, J=2.3 Hz, 1H), 6.28 (dd, J=8.9, 2.3 Hz, 1H), 3.96 (t, J=6.6 Hz, 2H), 3.66 (s, 3H), 3.47 (t, J=4.0 Hz, 4H), 2.28-2.35 (m, 6H), 1.81 (t, J=6.8 Hz, 2H).

MS (EI): m/z=425.0855 [M]⁺.

### Example 84

### Preparation of 5-(3,5-dihydroxybenzylidene)-1-methyl-3-(3-morpholinopropyl)-2-selenoxoimidazolidin-4-one (1age, Compound 84)

Dioxane (1.5 mL) was added to the compound (100 mg, 0.33 mmol) obtained in step 3) of Example 83 and 2,4-dihydroxybenzaldehyde (55 mg, 0.39 mmol) and followed by stirring, piperidine (49 μl) and aluminum chloride (8.8 mg) were added, and the mixture was warmed at 90° C. for 5 h. After the reaction, the mixture was subjected to column separation to obtain the target compound (42 mg, 30%).

¹H-NMR (300 MHz, DMSO-d₆) δ 9.45 (br s, 2H), 7.09 (d, J=2.0 Hz, 1H), 6.97 (s, 0.5H), 6.86 (s, 0.5H), 6.24-6.35 (m, 2H), 3.90-4.02 (m, 2H), 3.67 (s, 1.8H), 3.43-3.58 (m, 4H), 3.38 (s, 1.2H), 2.20-2.40 (m, 6H), 1.74-1.88 (m, 2H).

MS (EI): m/z=425.0854 [M]⁺.

### Example 85

### Preparation of 5-(4-chlorobenzylidene)-1-methyl-3-(3-morpholinopropyl)-2-selenoxoimidazolidin-4-one (1aqj, Compound 85)

Dioxane (0.8 mL) was added to the compound (52 mg, 0.17 mmol) obtained in step 3) of Example 81 and 4-chlorobenzaldehyde (19 mg, 0.13 mmol) and followed by stirring, piperidine (20 μl) and aluminum chloride (1.7 mg) were added, and the mixture was warmed at 90° C. for 5 h. After the reaction, the mixture was subjected to column separation to obtain the target compound (42 mg, 75%).

¹H-NMR (300 MHz, DMSO-d₆) δ 8.17 (d, J=8.6 Hz, 1.4H), 7.99 (d, J=8.6 Hz, 0.6H), 7.40-7.60 (m, 2H), 7.19 (s, 0.7H), 7.00 (s, 0.3H), 3.92-4.30 (m, 2H), 3.69 (s, 2.2H), 3.43-3.60 (m, 4H), 3.46 (s, 0.8H), 2.18-2.38 (m, 6H), 1.64-1.87 (m, 2H).

MS (EI): m/z=427.0565 [M]⁺.

### Example 86

### Preparation of 5-(4-fluorobenzylidene)-1-methyl-3-(3-morpholinopropyl)-2-selenoxoimidazolidin-4-one (1aqx, Compound 86)

Dioxane (2 mL) was added to the compound (100 mg, 0.33 mmol) obtained in step 3) of Example 81 and 4-fluorobenzaldehyde (35 uL, 0.33 mmol) and followed by stirring, piperidine (81 μl) and aluminum chloride (8.8 mg) were added, and the mixture was warmed at 90° C. for 5 h. After the reaction, the mixture was subjected to column separation to obtain the target compound (69 mg, 52%).

¹H-NMR (300 MHz, DMSO-d₆) δ 8.19-8.31 (m, 0.7H), 7.98-8.06 (m, 0.3H), 7.56-7.66 (m, 0.3H), 7.40-7.50 (m, 0.7H), 7.20-7.35 (m, 2H), 7.21 (s, 0.7H), 7.02 (s, 0.3H), 3.80-4.30 (m, 2H), 3.69 (s, 1.6H), 3.25-3.63 (m, 4H), 3.18 (s, 1.4H), 2.20-2.40 (m, 6H), 1.65-1.87 (m, 2H).

MS (EI): m/z=411.0859 [M]⁺.

### Example 87

### Preparation of 5-(2,4-dihydroxybenzylidene)-3-(5-fluorohexyl)-1-methyl-2-selenoxoimidazolidin-4-one (1ara, Compound 87)

### 1) Synthesis of N-(6-fluorohexyl)formamide

After 6-formamidohexylmethanesulfonate (800 mg, 3.6 mmol) was dissolved in acetonitrile (60 mL), TBAF (1 m soln. in THF, 7.17 mL, 7.17 mmol) was added, and the mixture for reaction was stirred for 2 hours. The reaction mixture was concentrated under reduced pressure, dried with water, ethyl acetate, and anhydrous sodium sulfate, then filtered, distilled under reduced pressure, dried in a high vacuum, and subjected to column separation to obtain the target compound (463 mg, 88%).

¹H-NMR (300 MHz, CDCl₃) δ 8.18 (s, 1H), 5.57 (br s, 1H), 4.52 (t, J=6.0 Hz, 1H), 4.37 (t, J=6.0 Hz, 1H), 3.15-3.40 (m, 2H), 1.30-1.80 (m, 8H).

MS (EI): m/z=146 [M−H]⁺.

### 2) Synthesis of N-(6-fluorohexyl) isoselenocyanate

After N-(6-fluorohexyl)formamide (595 mg, 4.0 mmol) was dissolved in MC (14 mL), triethylamine (2.4 mL, 17 mmol) and 4A MS (80 mg) were added. After triphosgene (648 mg, 2.2 mmol) was dissolved in MC (6 mL), the solution was slowly added dropwise over 1 hour. The reaction mixture was refluxed for 4 h, then selenium (638 mg, 8.1 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (513 mg, 61%).

¹H-NMR (300 MHz, CDCl₃) δ 4.54 (t, J=5.9 Hz, 1H), 4.38 (t, J=5.9 Hz, 1H), 3.63 (t, J=6.6 Hz, 2H), 1.60-1.85 (m, 4H), 1.35-1.57 (m, 4H).

MS (EI): m/z=207 [M−H]⁺.

### 3) Synthesis of 3-(6-fluorohexyl)-1-methyl-2-selenoxoimidazolidin-4-one

Dioxane (140 mL) and sarcosine (214 mg, 2.4 mmol) were added to the compound (495 mg, 1.37 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (450 mg, 68%).

¹H-NMR (300 MHz, CDCl₃) δ 4.47-4.57 (m, 1H), 4.33-4.41 (m, 1H), 3.88 (s, 2H), 3.83-3.97 (m, 2H), 3.42 (s, 2.4H), 3.33 (s, 0.6H), 1.57-1.82 (m, 4H), 1.32-1.54 (m, 4H).

MS (EI): m/z=280 [M+H]⁺.

### 4) Synthesis of 5-(2,4-dihydroxybenzylidene)-3-(5-fluorohexyl)-1-methyl-2-selenoxoimidazolidin-4-one (1ara)

Dioxane (0.4 mL) was added to the compound (40 mg, 0.14 mmol) obtained in step 3) and 2,4-dihydroxybenzaldehyde (20 mg, 0.14 mmol) and followed by stirring, piperidine (22 μl) and aluminum chloride (2 mg) were added, and the mixture was warmed at 90° C. for 5 h. After the reaction, the mixture was subjected to column separation to obtain the target compound (19 mg, 33%).

¹H-NMR (300 MHz, CD₃CN) δ 8.43 (d, J=8.8 Hz, 1H), 7.78 (br s, 2H), 7.17 (s, 1H), 6.41 (dd, J=8.8, 2.4 Hz, 1H), 6.36 (d, J=2.4 Hz, 1H), 4.50 (t, J=6.1 Hz, 1H), 4.34 (t, J=6.1 Hz, 1H), 3.65 (s, 2.7H), 3.37 (s, 0.3H).

MS (EI): m/z=400.0699 [M]⁺.

### Example 88

### Preparation of 5-(3,5-dihydroxybenzylidene)-3-(5-fluorohexyl)-1-methyl-2-selenoxoimidazolidin-4-one (1are, Compound 88)

Dioxane (0.4 mL) was added to the compound (40 mg, 0.14 mmol) obtained in step 3) of Example 87 and 3,5-dihydroxybenzaldehyde (20 mg, 0.14 mmol) and followed by stirring, piperidine (22 μl) and aluminum chloride (2 mg) were added, and the mixture was warmed at 90° C. for 5 h. After the reaction, the mixture was subjected to column separation to obtain the target compound (31 mg, 53%).

¹H-NMR (300 MHz, CD₃CN) δ 8.03/7.24 (1/9) (br s, 2H), 7.16/7.15/6.99/6.98 (0.8/0.8/0.2/0.2) (s, 2H), 6.88/6.78 (1/4) (s, 1H), 6.30-6.40 (m, 1H), 4.50 (t, J=6.1 Hz, 1H), 4.34 (t, J=6.1 Hz, 1H), 3.90-4.00 (m, 2H), 3.65 (s, 1H), 3.65 (s, 2.6H), 3.38 (s, 0.4H), 1.50-1.78 (m, 4H), 1.28-1.50 (m, 4H).

MS (EI): m/z=400.0702 [M]⁺.

### Example 89

### Preparation of 5-(2,4-dihydroxybenzylidene)-3-(5-fluoropentyl)-1-methyl-2-selenoxoimidazolidin-4-one (1asa, Compound 89)

### 1) Synthesis of N-(5-fluoropentyl)formamide

After 5-formamidopentyl methanesulfonate (3,460 mg, 17 mmol) was dissolved in acetonitrile (270 mL), TBAF (1 m soln. in THF, 23 mL, 23 mmol) was added, and the mixture for reaction was stirred at 80° C. for 2 h. The reaction mixture was concentrated under reduced pressure, dried with water, ethyl acetate, and anhydrous sodium sulfate, then filtered, distilled under reduced pressure, dried in a high vacuum, and subjected to column separation to obtain the target compound (1,820 mg, 83%).

¹H-NMR (300 MHz, CDCl₃) δ 8.17 (s, 1H), 5.75 (br s, 1H), 4.53 (t, J=5.9 Hz, 1H), 4.37 (t, J=5.9 Hz, 1H), 3.17-3.40 (m, 2H), 1.33-1.86 (m, 6H).

MS (EI): m/z=148 [M+H]⁺.

### 2) Synthesis of N-(5-fluoropentyl) isoselenocyanate

After N-(5-fluoropentyl)amide (824 mg, 6.2 mmol) was dissolved in EDC (22 mL), triethylamine (3.7 mL, 26 mmol) and 4A MS (124 mg) were added. After triphosgene (992 mg, 3.3 mmol) was dissolved in EDC (11 mL), the solution was slowly added dropwise over 1 hour. The reaction mixture was refluxed for 4 h, then selenium (977 mg, 12 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (820 mg, 68%).

¹H-NMR (300 MHz, CDCl₃) δ 4.56 (t, J=5.8 Hz, 1H), 4.40 (t, J=5.8 Hz, 1H), 3.65 (t, J=6.5 Hz, 2H), 1.50-1.90 (m, 6H).

MS (EI): m/z=195 [M+H]⁺.

### 3) Synthesis of 3-(5-fluoropentyl)-1-methyl-2-selenoxoimidazolidin-4-one

Dioxane (200 mL) and sarcosine (372 mg, 4.2 mmol) were added to the compound (810 mg, 4.17 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (194 mg, 17%).

¹H-NMR (300 MHz, CDCl₃) δ 4.53 (t, J=6.1 Hz, 1H), 4.37 (t, J=6.1 Hz, 1H), 3.91 (t, J=7.5 Hz, 1H), 3.87 (s, 1.7H), 3.71 (s, 0.3H), 3.42 (s, 3H), 1.60-1.87 (m, 4H), 1.37-1.54 (m, 2H).

MS (EI): m/z=266 [M+H]⁺.

### 4) Preparation of 5-(2,4-dihydroxybenzylidene)-3-(5-fluoropentyl)-1-methyl-2-selenoxoimidazolidin-4-one (1asa)

Dioxane (0.4 mL) was added to the compound (38 mg, 0.14 mmol) obtained in step 3) and 2,4-dihydroxybenzaldehyde (20 mg, 0.14 mmol) and followed by stirring, piperidine (22 μl) and aluminum chloride (2 mg) were added, and the mixture was warmed at 90° C. for 5 h. After the reaction, the mixture was subjected to column separation to obtain the target compound (39 mg, 70%).

¹H-NMR (300 MHz, CD3CN) δ 8.41 (d, J=8.7 Hz, 1H), 7.75 (br s, 2H), 7.17 (s, 1H), 6.41 (dd, J=8.7, 2.4 Hz, 1H), 6.36 (d, J=2.4 Hz, 1H), 4.51 (t, J=6.1 Hz, 1H), 4.36 (t, J=6.1 Hz, 1H), 3.97 (t, J=7.3 Hz, 2H), 3.65 (s, 2.7H), 3.42 (s, 0.3H), 1.59-1.81 (m, 4H), 1.33-1.48 (m, 2H).

MS (EI): m/z=386.0544 [M]⁺.

### Example 90

### Preparation of 5-(3,5-dihydroxybenzylidene)-3-(5-fluoropentyl)-1-methyl-2-selenoxoimidazolidin-4-one (1ase, Compound 90)

Dioxane (0.4 mL) was added to the compound (38 mg, 0.14 mmol) obtained in step 3) of Example 89 and 2,4-dihydroxybenzaldehyde (20 mg, 0.14 mmol) and followed by stirring, piperidine (22 μl) and aluminum chloride (2 mg) were added, and the mixture was warmed at 90° C. for 5 h. After the reaction, the mixture was subjected to column separation to obtain the target compound (29 mg, 52%).

¹H-NMR (300 MHz, DMSO-d₆) δ 9.52 (s, 1H), 9.42 (s, 1H), 7.09 (d, J=2.0 Hz, 1H), 6.97 (s, 0.5H), 6.87 (s, 0.5H), 6.22-6.38 (m, 2H), 4.51 (dt, J=6.0, 1.7 Hz, 1H), 4.35 (dt, J=6.0, 1.7 Hz, 1H), 3.85-3.97 (m, 2H), 3.67 (s, 2H), 3.38 (s, 1H), 1.52-1.81 (m, 4H), 1.27-1.46 (m, 2H).

MS (EI): m/z=386.0548 [M]⁺.

### Example 91

### Preparation of 3-butyl-5-(2,4-dihydroxybenzylidene)-1-methyl-2-selenoxoimidazolidin-4-one (1ata, Compound 91)

### 1) Synthesis of N-butylformamide

After N-butylamine (5.0 mL 51 mmol) was added to formic acid (5.7 mL, 152 mmol), the reaction was conducted under reflux at 90° C. for 48 hours and terminated. The reaction mixture was concentrated under reduced pressure, dried in a high vacuum, and subjected to column separation to obtain the target compound (787 mg, 15%).

¹H-NMR (300 MHz, CDCl₃) δ 8.15 (s, 1H), 6.06 (br s, 1H), 3.29 (q, J=6.9 Hz, 2H), 1.52 (quintet, J=7.0 Hz, 2H), 1.36 (sextet, J=7.0 Hz, 2H), 0.93 (t, J=7.2 Hz, 3H).

### 2) Synthesis of N-butyl isoselenocyanate

After N-butylformamide (370 mg, 3.7 mmol) was dissolved in EDC (12 mL), triethylamine (2.2 mL, 16 mmol) and 4A MS (80 mg) were added. After triphosgene (586 mg, 2.0 mmol) was dissolved in EDC (8 mL), the solution was slowly added dropwise over 1 hour. The reaction mixture was refluxed for 4 h, then selenium (578 mg, 7.3 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (368 mg, 62%).

¹H-NMR (300 MHz, CDCl₃) δ 3.62 (t, J=6.6 Hz, 2H), 1.73 (quintet, J=7.1 Hz, 2H), 1.47 (sextet, J=7.5 Hz, 2H), 0.96 (t, J=7.3 Hz, 3H).

### 3) Synthesis of 3-butyl-1-methyl-2-selenoxoimidazolidin-4-one

Dioxane (30 mL) and sarcosine (202 mg, 2.3 mmol) were added to the compound (368 mg, 2.3 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (255 mg, 48%).

¹H-NMR (300 MHz, CDCl₃) δ 3.87-3.92 (m, 4H), 3.43 (s, 3H), 1.62-1.72 (m, 2H), 1.37 (sestet, J=7.5 Hz, 2H), 0.95 (t, J=7.3 Hz, 3H).

MS (EI): m/z=234 [M+H]⁺.

### 4) Synthesis of 3-butyl-5-(2,4-dihydroxybenzylidene)-1-methyl-2-selenoxoimidazolidin-4-one (1ata)

Dioxane (1.0 mL) was added to the compound (50 mg, 0.21 mmol) obtained in step 3) and 2,4-dihydroxybenzaldehyde (30 mg, 0.21 mmol) and followed by stirring, piperidine (32 μl) and aluminum chloride (2.8 mg) were added, and the mixture was warmed at 90° C. for 5 h. After the reaction, the mixture was subjected to column separation to obtain the target compound (10 mg, 13%).

¹H-NMR (300 MHz, CD₃CN) δ 8.41 (d, J=8.7 Hz, 1H), 7.18 (s, 1H), 6.27-6.45 (m, 2H), 3.95 (t, J=7.4 Hz, 2H), 3.65 (s, 3H), 1.50-1.74 (m, 2H), 1.20-1.40 (m, 2H), 0.93 (t, J=7.3 Hz, 3H).

MS (EI): m/z=354.0484 [M]⁺.

### Example 92

### Preparation of 3-butyl-5-(3,5-dihydroxybenzylidene)-1-methyl-2-selenoxoimidazolidin-4-one (1ate, Compound 92)

Dioxane (0.8 mL) was added to the compound (30 mg, 0.13 mmol) obtained in step 3) of Example 91 and 2,4-dihydroxybenzaldehyde (18 mg, 0.13 mmol) and followed by stirring, piperidine (19 μl) and aluminum chloride (1.7 mg) were added, and the mixture was warmed at 90° C. for 5 h. After the reaction, the mixture was subjected to column separation to obtain the target compound (31 mg, 68%).

¹H-NMR (300 MHz, CD₃CN) δ 7.52 (br s, 2H), 7.15 (d, J=2.0 Hz, 1H), 6.88 (s, 0.4H), 6.78 (s, 0.6H), 6.30-6.40 (m, 2H), 3.89-4.00 (m, 2H), 3.65 (s, 2H), 3.38 (s, 1H), 1.57-1.74 (m, 2H), 1.22-1.42 (m, 2H), 0.85-1.00 (m, 3H).

MS (EI): m/z=354.0479 [M]⁺.

### Example 93

### Preparation of 5-(2,4-dihydroxybenzylidene)-1-methyl-3-pentyl-2-selenoxoimidazolidin-4-one (1aua, Compound 93)

### 1) Synthesis of N-pentylformamide

After N-amylamine (5.0 mL 43 mmol) was added to formic acid (4.9 mL, 129 mmol), the reaction was conducted under reflux at 90° C. for 48 hours and terminated. The reaction mixture was concentrated under reduced pressure, dried in a high vacuum, and subjected to column separation to obtain the target compound (2.98 g, 60%).

¹H-NMR (300 MHz, CDCl₃) δ 8.15 (s, 1H), 6.17 (br s, 1H), 3.28 (q, J=6.8 Hz, 2H), 1.53 (quintet, J=6.8 Hz, 2H), 1.29-1.34 (m, 6H), 0.88-0.92 (m, 3H).

MS (EI): m/z=114 [M−H]⁺.

### 2) Synthesis of N-pentyl isoselenocyanate

After N-pentylformamide (1.0 g, 3.7 mmol) was dissolved in EDC (30 mL), triethylamine (5.2 mL, 37 mmol) and 4A MS (200 mg) were added. After triphosgene (1.39 g, 4.7 mmol) was dissolved in EDC (20 mL), the solution was slowly added dropwise over 1 hour. The reaction mixture was refluxed for 4 h, then selenium (1.37 mg, 7.3 mmol) was added, and stirring was performed for 4 h. The reaction mixture was cooled, then washed with water, methylene chloride, and brine, dried over anhydrous sodium sulfate, filtered, concentrated under reduced pressure, and then subjected to column separation to obtain the target compound (1.06 g, 69%).

¹H-NMR (300 MHz, CDCl₃) δ 3.61 (t, J=6.6 Hz, 2H), 1.74 (quintet, J=6.6 Hz, 2H), 1.30-1.46 (m, 4H), 0.95 (t, J=6.6 Hz, 1H).

### 3) Synthesis of 1-methyl-3-pentyl-2-selenoxoimidazolidin-4-one

Dioxane (80 mL) and sarcosine (536 mg, 6.0 mmol) were added to the compound (1.06 g, 6.0 mmol) obtained in step 2), and the mixture was warmed at 110° C. for 4 h. After the reaction, the reaction mixture was cooled to room temperature and concentrated under reduced pressure, and the resulting residue was subjected to column separation to obtain the target compound (622 mg, 42%).

¹H-NMR (300 MHz, CDCl₃) δ 3.87-3.91 (m, 4H), 3.42 (s, 3H), 1.69 (quintet, J=7.5 Hz, 2H), 1.26-1.40 (m, 4H), 0.90 (t, J=6.8 Hz, 3H).

MS (EI): m/z=248 [M]⁺.

### 4) Synthesis of 5-(2,4-dihydroxybenzylidene)-1-methyl-3-pentyl-2-selenoxoimidazolidin-4-one (1aua)

Dioxane (1.0 mL) was added to the compound (50 mg, 0.20 mmol) obtained in step 3) and 2,4-dihydroxybenzaldehyde (28 mg, 0.20 mmol) and followed by stirring, piperidine (30 μl) and aluminum chloride (2.7 mg) were added, and the mixture was warmed at 90° C. for 5 h. After the reaction, the mixture was subjected to column separation to obtain the target compound (26 mg, 35%).

¹H-NMR (300 MHz, CD₃CN) δ 8.43 (d, J=8.7 Hz, 1H), 7.18 (br s, 1H), 6.27-6.45 (m, 2H), 3.94 (t, J=7.4 Hz, 2H), 3.65 (s, 3H), 3.42 (s, 0.3H), 1.60-1.78 (m, 2H), 1.20-1.40 (m, 4H), 0.80-0.95 (m, 3H).

MS (EI): m/z=368.0641 [M]⁺.

### Example 94

### Preparation of 5-(3,5-dihydroxybenzylidene)-1-methyl-3-pentyl-2-selenoxoimidazolidin-4-one (1aue, Compound 94)

Dioxane (1.0 mL) was added to the compound (30 mg, 0.12 mmol) obtained in step 3) of Example 93 and 3,5-dihydroxybenzaldehyde (17 mg, 0.12 mmol) and followed by stirring, piperidine (18 μl) and aluminum chloride (1.6 mg) were added, and the mixture was warmed at 90° C. for 5 h. After the reaction, the mixture was subjected to column separation to obtain the target compound (38 mg, 84%).

¹H-NMR (300 MHz, CD3CN) δ 7.15 (d, J=2.1 Hz, 1H), 6.88 (s, 0.3H), 6.78 (s, 0.7H), 6.28-6.39 (m, 2H), 3.89-4.00 (m, 2H), 3.65 (s, 1.9H), 3.38 (s, 1.1H), 1.62-1.75 (m, 2H), 1.20-1.48 (m, 4H), 0.84-0.94 (m, 3H).

MS (EI): m/z=368.0638 [M]⁺.

### Example 95

### Preparation of 5-(2,4-dihydroxybenzylidene)-1-ethyl-3-phenyl-2-selenoxoimidazolidin-4-one (1baa, Compound 95)

### 1) Synthesis of 1-ethyl-3-phenyl-2-selenoxoimidazolidin-4-one

Dioxane (170 mL) and N-ethylglycine (401 mg, 3.89 mmol) were added to the compound (817 mg, 3.89 mmol) obtained in step 2) of Example 1, and the mixture was warmed at 110° C. for 4 h. After the reaction, the reaction mixture was cooled to room temperature and concentrated under reduced pressure, and the resulting residue was subjected to column separation to obtain the target compound (210 mg, 87%).

¹H-NMR (300 MHz, CDCl₃) δ 7.42-7.56 (m, 3H), 7.28-7.36 (m, 2H), 4.04 (q, J=7.2 Hz, 2H), 4.02 (s, 2H), 1.36 (t, J=7.2 Hz, 3H).

MS (EI): m/z=268 [M]⁺.

### 2) Synthesis of 5-(2,4-dihydroxybenzylidene)-1-ethyl-3-phenyl-2-selenoxoimidazolidin-4-one (1baa)

Dioxane (0.6 mL) was added to the compound (52 mg, 0.2 mmol) obtained in step 1) and 2,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (63 mg, 81%).

¹H-NMR (300 MHz, DMSO-d₆) δ 10.4 (s, 1H), 10.1 (s, 1H), 8.46 (d, J=8.7 Hz, 1H), 7.25-7.58 (m, 5H), 6.38 (s, 1H), 6.25 (d, J=9.0 Hz, 1H), 4.35 (q, J=6.9 Hz, 2H), 1.15-1.35 (m, 3H).

MS (ESI): m/z=389.0401 [M+H]⁺.

### Example 96

### Preparation of 5-(2,3-dihydroxybenzylidene)-1-ethyl-3-phenyl-2-selenoxoimidazolidin-4-one (1bab, Compound 96)

Dioxane (0.6 mL) was added to the compound (52 mg, 0.2 mmol) obtained in step 1) of Example 95 and 2,3-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (25 mg, 32%).

¹H-NMR (300 MHz, DMSO-d₆) δ 9.40 (br s, 2H), 7.69-7.78 (m, 1H), 7.43-7.57 (m, 5H), 7.35-7.42 (m, 2H), 7.30-7.35 (m, 1H), 4.30-4.43 (m, 1.5H), 4.15-4.28 (m, 0.5H), 1.31 (t, J=6.9 Hz, 2H), 0.87 (t, 6.9 Hz, 1H).

MS (ESI): m/z=411.0222 [M+Na]⁺.

### Example 97

### Preparation of 5-(4-(dimethylamino)benzylidene)-1-ethyl-3-phenyl-2-selenoxoimidazolidin-4-one (1bac, Compound 97)

Dioxane (0.6 mL) was added to the compound (52 mg, 0.2 mmol) obtained in step 1) of Example 95 and 4-dimethylaminobenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (16 mg, 22%).

¹H-NMR (300 MHz, DMSO-d₆) δ 8.26 (d, J=1.7 Hz, 2H), 7.42-7.42 (m, 1H), 7.19 (s, 0.7H), 7.06 (s, 0.3H), 6.79 (d, J=8.6 Hz, 0.8H), 6.74 (d, J=9.2 Hz, 1.2H), 4.34-4.50 (m, 2H), 3.10 (s, 4H), 3.01 (s, 2H), 1.18-1.35 (m, 3H).

MS (ESI): m/z=400.0929 [M+H]⁺.

### Example 98

### Preparation of 5-(3,4-dihydroxybenzylidene)-1-ethyl-3-phenyl-2-selenoxoimidazolidin-4-one (1bad, Compound 98)

Dioxane (0.6 mL) was added to the compound (52 mg, 0.2 mmol) obtained in step 1) of Example 95 and 3,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (31 mg, 40%).

¹H-NMR (300 MHz, DMSO-d₆) δ 9.69 (s, 1H), 7.98 (d, J=1.7 Hz, 1H), 7.42-7.58 (m, 4H), 7.33-7.41 (m, 2H), 7.20-7.29 (m, 1H), 7.13 (s, 1H), 6.89 (d, J=8.0 Hz, 0.25H), 6.76 (d, J=8.3 Hz, 0.75H), 4.29-4.60 (m, 2H), 1.16-1.38 (m, 3H).

MS (ESI): m/z=389.0404 [M+H]⁺.

### Example 99

### Preparation of 5-(3,5-dihydroxybenzylidene)-1-ethyl-3-phenyl-2-selenoxoimidazolidin-4-one (1bae, Compound 99)

Dioxane (0.6 mL) was added to the compound (52 mg, 0.2 mmol) obtained in step 1) of Example 95 and 3,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (38 mg, 49%).

¹H-NMR (300 MHz, DMSO-d₆) δ 9.60 (s, 0.5H), 9.40 (s, 1.5H), 7.32-7.80 (m, 5H), 7.09 (s, 2H), 6.31 (s, 2H), 4.30-4.55 (m, 1.5H), 4.14-4.29 (m, 0.5H), 1.22-1.40 (m, 3H).

MS (ESI): m/z=389.0403 [M+H]⁺.

### Example 100

### Preparation of 5-(3,5-dihydroxybenzylidene)-1-ethyl-3-(4-ethylphenyl)-2-selenoxoimidazolidin-4-one (1bce, Compound 100)

### 1) Synthesis of 3-(4-ethylphenyl)-1-ethyl-2-selenoxoimidazolidin-4-one

Dioxane (240 mL) and N-ethylglycine (400 mg, 3.88 mmol) were added to the compound (815 mg, 3.88 mmol) obtained in step 2) of Example 24, and the mixture was warmed at 110° C. for 4 h. After the reaction, the reaction mixture was cooled to room temperature and concentrated under reduced pressure, and the resulting residue was subjected to column separation to obtain the target compound (520 mg, 478%).

¹H-NMR (300 MHz, CDCl₃) δ 7.33 (d, J=8.0 Hz, 2H), 7.22 (d, J=7.7 Hz, 2H), 4.05 (q, J=7.1 Hz, 2H), 4.03 (s, 2H), 2.71 (q, J=7.6 Hz, 2H), 1.31 (t, J=7.2 Hz, 3H), 1.27 (t, J=7.2 Hz, 3H).

MS (EI): m/z=296 [M+H]⁺.

### 2) Synthesis of 5-(3,5-dihydroxybenzylidene)-1-ethyl-3-(4-ethylphenyl)-2-selenoxoimidazolidin-4-one (1bce)

Dioxane (0.6 mL) was added to the compound (59 mg, 0.2 mmol) obtained in step 1) and 2,4-dihydroxybenzaldehyde (29 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (40 mg, 50%).

¹H-NMR (300 MHz, CDCl₃) δ 7.13-7.41 (m, 6H), 6.98 (s, 0.3H), 6.88 (s, 0.7H), 6.31-6.44 (m, 1H), 4.38 (q, J=7.1 Hz, 1.3H), 4.26 (q, J=7.1 Hz, 0.7H), 2.62-2.80 (m, 2H), 1.13-1.44 (m, 6H).

MS (ESI): m/z=416.0642 [M]⁺.

### Example 101

### Preparation of 5-(4-bromobenzylidene)-1-ethyl-3-(4-ethylphenyl)-2-selenoxoimidazolidin-4-one (1bci, Compound 101)

Dioxane (0.6 mL) was added to the compound (61 mg, 0.2 mmol) obtained in step 1) of Example 100 and 4-bromobenzaldehyde (41 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (20 mg, 22%).

¹H-NMR (300 MHz, CDCl₃) δ 8.25 (d, J=8.9 Hz, 1H), 7.42-7.59 (m, 4H), 7.29-7.41 (m, 2H), 7.15 (s, 0.6H), 7.01 (s, 0.4H), 6.50-6.67 (m, 2H), 3.78 (s, 1.8H), 3.60 (s, 1.2H), 3.34 (br s, 4H), 1.90-2.05 (m, 4H).

MS (ESI): m/z=487.9739 [M+Na]⁺.

### Example 102

### Preparation of 1-ethyl-3-(4-ethylphenyl)-5-(4-pyrrolidin-1-yl)benzylidene-2-selenoxoimidazolidin-4-one (1bck, Compound 102)

Dioxane (0.6 mL) was added to the compound (59 mg, 0.2 mmol) obtained in step 1) of Example 100 and 4-(pyrrolidin-1-yl)benzaldehyde (39 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (25 mg, 28%).

¹H-NMR (300 MHz, CDCl₃) δ 8.21 (d, J=8.8 Hz, 1H), 7.46-7.77 (m, 0.4H), 7.19-7.45 (m, 5H), 7.17 (s, 0.4H), 6.77 (s, 0.6H), 6.57 (d, J=8.8 Hz, 0.6H), 6.52 (d, J=8.6 Hz, 1H), 4.45 (pentet, J=7.2 Hz, 1.6H), 4.20-4.39 (m, 0.4H), 3.25-3.55 (m, 4H), 2.71 (q, J=7.5 Hz, 2H), 1.93-2.20 (m, 4H), 1.42 (t, J=7.2 Hz, 2H), 1.27 (t, J=7.6 Hz, 3H), 1.08 (t, J=7.0 Hz, 1H).

MS (EI): m/z=453.1317 [M]⁺.

### Example 103

### Preparation of 1-benzyl-5-(2,4-dihydroxybenzylidene)-3-phenyl-2-selenoxoimidazolidin-4-one (1caa, Compound 103)

### 1) Synthesis of 1-benzyl-3-phenyl-2-selenoxoimidazolidin-4-one

Dioxane (170 mL) and N-benzylglycine (643 mg, 3.89 mmol) were added to the compound (817 mg, 3.89 mmol) obtained in step 2) of Example 1, and the mixture was warmed at 110° C. for 4 h. After the reaction, the reaction mixture was cooled to room temperature and concentrated under reduced pressure, and the resulting residue was subjected to column separation to obtain the target compound (1.23 g, 96%).

¹H-NMR (300 MHz, CDCl₃) δ 7.28-7.56 (m, 10H), 5.20 (s, 2H), 3.89 (s, 2H).

MS (EI): m/z=330 [M+H]⁺.

### 2) Synthesis of 1-benzyl-5-(2,4-dihydroxybenzylidene)-3-phenyl-2-selenoxoimidazolidin-4-one (1caa)

Dioxane (0.6 mL) was added to the compound (66 mg, 0.2 mmol) obtained in step 1) and 2,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (46 mg, 51%).

¹H-NMR (300 MHz, DMSO-d₆) δ 10.3 (s, 1H), 10.1 (s, 1H), 8.51 (d, J=8.8 Hz, 1H), 7.28-7.59 (m, 10H), 7.27 (s, 1H), 6.26 (s, 1H), 6.20 (d, J=8.7 Hz, 1H), 5.64 (s, 2H).

MS (ESI): m/z=451.0560 [M+H]⁺.

### Example 104

### Preparation of 1-benzyl-5-(2,3-dihydroxybenzylidene)-3-phenyl-2-selenoxoimidazolidin-4-one (1cab, Compound 104)

Dioxane (0.6 mL) was added to the compound (66 mg, 0.2 mmol) obtained in step 1) of Example 103 and 2,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (41 mg, 46%).

¹H-NMR (300 MHz, DMSO-d₆) δ 9.10 (br s, 2H), 7.77 (d, J=6.8 Hz, 1H), 7.32-7.58 (m, 9H), 7.15 (s, 1H), 6.68 (d, J=7.8 Hz, 1H), 668 (d, J=1.4 Hz, 1H), 6.52-6.67 (m, 1H), 5.64 (s, 2H).

MS (ESI): m/z=451.0559 [M+H]⁺.

### Example 105

### Preparation of 1-benzyl-5-(4-(dimethylamino)benzylidene)-3-phenyl-2-selenoxoimidazolidin-4-one (1cac, Compound 105)

Dioxane (0.6 mL) was added to the compound (66 mg, 0.2 mmol) obtained in step 1) of Example 103 and 4-(dimethylamino)benzaldehyde (33 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (59 mg, 64%).

¹H-NMR (300 MHz, DMSO-d₆) δ 8.03 (d, J=8.9 Hz, 2H), 7.29-7.47 (m, 9H), 6.69 (s, 1H), 6.56 (d, J=8.8 Hz, 2H), 5.66 (s, 2H), 2.48 (s, 6H).

MS (ESI): m/z=462.1081 [M+H]⁺.

### Example 106

### Preparation of 1-benzyl-5-(3,4-dihydroxybenzylidene)-3-phenyl-2-selenoxoimidazolidin-4-one (1cad, Compound 106)

Dioxane (0.6 mL) was added to the compound (66 mg, 0.2 mmol) obtained in step 1) of Example 103 and 3,4-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (48 mg, 53%).

¹H-NMR (300 MHz, DMSO-d₆) δ 9.79 (br s, 1H), 9.22 (br s, 1H), 7.81 (d, J=1.9 Hz, 1H), 7.10-7.62 (m, 11H), 6.91 (s, 1H), 6.70 (d, J=8.4 Hz, 1H), 5.65 (s, 2H).

MS (ESI): m/z=451.0558 [M+H]⁺.

### Example 107

### Preparation of 1-benzyl-5-(3,5-dihydroxybenzylidene)-3-phenyl-2-selenoxoimidazolidin-4-one (1cae, Compound 107)

Dioxane (0.6 mL) was added to the compound (66 mg, 0.2 mmol) obtained in step 1) of Example 103 and 3,5-dihydroxybenzaldehyde (25 mg, 0.22 mmol) and followed by stirring, piperidine (30 μl) and aluminum chloride (3 mg) were added, and the mixture was warmed at 90° C. for 5 h. After the reaction, the mixture was subjected to column separation to obtain the target compound (72 mg, 80%).

¹H-NMR (300 MHz, DMSO-d₆) δ 9.45, 9.42, 9.38, 9.25 (each s, 2H), 7.22-7.60 (m, 6H), 6.64-6.90 (m, 3H), 6.12-6.35 (m, 1H), 6.08 (s, 2H), 5.68 (s, 0.4H), 5.54 (s, 1H), 5.00, 4.86 (each s, 0.6H).

MS (ESI): m/z=473.0378 [M+Na]⁺.

### Example 108

### Preparation of 1-benzyl-5-(2,4-dihydroxybenzylidene)-3-(4-ethylphenyl)-2-selenoxoimidazolidin-4-one (1cca, Compound 108)

### 1) Synthesis of 3-(4-ethylphenyl)-1-benzyl-2-selenoxoimidazolidin-4-one

Dioxane (15 mL) and N-benzylglycine (864 mg, 5.23 mmol) were added to the compound (1.10 g, 5.23 mmol) obtained in step 2) of Example 24, and the mixture was warmed at 110° C. for 4 h. After the reaction, the reaction mixture was cooled to room temperature and concentrated under reduced pressure, and the resulting residue was subjected to column separation to obtain the target compound (1.72 g, 92%).

¹H-NMR (300 MHz, CDCl₃) δ 7.38-7.49 (m, 5H), 7.34 (d, J=8.5 Hz, 2H), 7.25 (d, J=8.1 Hz, 2H), 5.22 (s, 2H), 3.87 (s, 2H), 2.72 (q, J=7.6 Hz, 2H), 1.28 (t, J=7.6 Hz, 3H).

MS (EI): m/z=329 [M]⁺.

### 2) Synthesis of 1-benzyl-5-(2,4-dihydroxybenzylidene)-3-(4-ethylphenyl)-2-selenoxoimidazolidin-4-one (1cca)

Dioxane (0.9 mL) was added to the compound (100 mg, 0.28 mmol) obtained in step 1) and 2,4-dihydroxybenzaldehyde (43 mg, 0.31 mmol) and followed by stirring, piperidine (41 μl) and aluminum chloride (4 mg) were added, and the mixture was warmed at 90° C. for 5 h. After the reaction, the mixture was subjected to column separation to obtain the target compound (31 mg, 23%).

¹H-NMR (300 MHz, DMSO-d₆) δ 10.2 (br s, 1H), 10.1 (br s, 1H), 8.50 (d, J=8.9 Hz, 1H), 7.28-7.50 (m, 9H), 7.25 (s, 1H), 6.23-6.28 (m, 1H), 6.13-6.22 (m, 1H), 5.63 (s, 2H), 2.56-2.76 (m, 2H), 1.10-1.30 (m, 3H).

MS (EI): m/z=478.0793 [M]⁺.

### Example 109

### Preparation of 1-benzyl-5-(3,4-dihydroxybenzylidene)-3-(4-ethylphenyl)-2-selenoxoimidazolidin-4-one (1ccd, Compound 109)

Dioxane (0.9 mL) was added to the compound (100 mg, 0.28 mmol) obtained in step 1) of Example 108 and 3,4-dihydroxybenzaldehyde (43 mg, 0.31 mmol) and followed by stirring, piperidine (41 μl) and aluminum chloride (4 mg) were added, and the mixture was warmed at 90° C. for 5 h. After the reaction, the mixture was subjected to column separation to obtain the target compound (73 mg, 55%).

¹H-NMR (300 MHz, DMSO-d₆) δ 9.70 (br s, 1H), 9.20 (br s, 1H), 7.80 (d, J=2.1 Hz, 1H), 7.10-7.49 (m, 10H), 6.90 (s, 1H), 6.70 (d, J=8.3 Hz, 1H), 5.68 (s, 2H), 267 (q, J=7.6 Hz, 2H), 1.22 (t, J=7.6 Hz, 3H).

MS (EI): m/z=478.0798 [M]⁺.

### Example 110

### Preparation of 1-benzyl-5-(3,5-dihydroxybenzylidene)-3-(4-ethylphenyl)-2-selenoxoimidazolidin-4-one (1cce, Compound 110)

Dioxane (0.9 mL) was added to the compound (100 mg, 0.28 mmol) obtained in step 1) of Example 108 and 3,5-dihydroxybenzaldehyde (43 mg, 0.22 mmol) and followed by stirring, piperidine (41 μl) and aluminum chloride (4 mg) were added, and the mixture was warmed at 90° C. for 5 h. After the reaction, the mixture was subjected to column separation to obtain the target compound (67 mg, 50%).

¹H-NMR (300 MHz, DMSO-d₆) δ 9.45 (s, 1H), 9.26 (s, 1H), 7.23-7.53 (m, 7H), 7.10-7.20 (m, 1H), 6.65-6.96 (m, 2H), 6.03-6.35 (m, 2H), 5.67 (s, 0.5H), 5.54 (s, 0.5H), 4.99 (s, 0.5H), 4.86 (s, 0.5H), 2.57-2.66 (m, 2H), 1.06-1.30 (m, 3H).

MS (EI+): m/z=478.0797 [M]⁺.

### Example 111

### Preparation of 1-benzyl-3-(4-chlorophenyl)-5-(2,3-dihydroxybenzylidene)-2-selenoxoimidazolidin-4-one (1cdb, Compound 111)

### 1) Synthesis of 3-(4-chlorophenyl)-1-benzyl-2-selenoxoimidazolidin-4-one

Dioxane (11 mL) and N-benzylglycine (610 mg, 3.69 mmol) were added to the compound (800 mg, 3.69 mmol) obtained in step 2) of Example 35, and the mixture was warmed at 110° C. for 4 h. After the reaction, the reaction mixture was cooled to room temperature and concentrated under reduced pressure, and the resulting residue was subjected to column separation to obtain the target compound (1.27 g, 95%).

¹H-NMR (300 MHz, CDCl₃) δ 7.28-7.55 (m, 9H), 5.21 (s, 2H), 3.88 (s, 2H).

MS (EI): m/z=364 [M+H]⁺.

### 2) Synthesis of 1-benzyl-3-(4-chlorophenyl)-5-(2,3-dihydroxybenzylidene)-2-selenoxoimidazolidin-4-one (1cdb)

Dioxane (0.9 mL) was added to the compound (98 mg, 0.27 mmol) obtained in step 1) and 2,3-dihydroxybenzaldehyde (41 mg, 0.30 mmol) and followed by stirring, piperidine (41 μl) and aluminum chloride (4 mg) were added, and the mixture was warmed at 90° C. for 5 h. After the reaction, the mixture was subjected to column separation to obtain the target compound (49 mg, 38%).

¹H-NMR (300 MHz, DMSO-d₆) δ 9.56 (br s, 1H), 9.16 (br s, 1H), 7.48-7.65 (m, 5H), 7.25-7.47 (m, 4H), 7.24 (s, 1H), 6.80 (dd, J=7.8, 1.4 Hz, 1H), 6.55 (t, J=7.8 Hz, 1H), 5.63 (s, 2H).

MS (EI+): m/z=484.0097 [M]⁺.

### Example 112

### Preparation of 1-benzyl-3-(4-chlorophenyl)-5-(3,4-dihydroxybenzylidene)-2-selenoxoimidazolidin-4-one (1cdd, Compound 112)

Dioxane (0.9 mL) was added to the compound (98 mg, 0.27 mmol) obtained in step 1) of Example 11 and 3,4-dihydroxybenzaldehyde (41 mg, 0.30 mmol) and followed by stirring, piperidine (41 μl) and aluminum chloride (4 mg) were added, and the mixture was warmed at 90° C. for 5 h. After the reaction, the mixture was subjected to column separation to obtain the target compound (88 mg, 67%).

¹H-NMR (300 MHz, DMSO-d₆) δ 9.50 (br s, 2H), 7.81 (s, 1H), 7.20-7.65 (m, 8H), 6.91 (s, 1H), 6.72 (d, J=8.3 Hz, 1H), 5.68 (s, 2H).

MS (EI+): m/z=484.0096 [M]⁺.

### Example 113

### Preparation of 1-methyl-3-phenyl-5-(4-(pyrrolidin-1-yl)benzylidene)-2-selenoxoimidazolidin-4-one·hydrochloride (1aak·HCl, Compound 113)

After 1-methyl-3-phenyl-5-(4-(pyrrolidin-1-yl)benzylidene)-2-selenoxoimidazolidin-4-one (1aak, 10 mg) was dissolved in dichloromethane (0.3 mL), 2 M HCl/ether (0.3 mL) was added dropwise in an ice bath at 0° C. After stirring at room temperature for 3 hours, drying under reduced pressure was performed to obtain the target compound (8.4 mg, ˜78%).

¹H-NMR (300 MHz, CDCl₃) δ 8.23 (br s, 2H), 7.27-7.70 (m, 5H), 6.80 (br s, 1H), 6.54 (br s, 2H), 3.90 (br s, 3H), 3.39 (br s, 4H), 2.04 (br s, 4H).

MS (ESI): m/z=412 [M+H]⁺.

### Example 114

### Preparation of 1-methyl-5-(4-pyrrolidin-1-yl)benzylidene-2-selenoxo-3-(4-tolyl)-imidazolidin-4-one·hydrochloride (1abk·HCl, Compound 114)

After 1-methyl-5-(4-pyrrolidin-1-yl)benzylidene-2-selenoxo-3-(4-tolyl)-imidazolidin-4-one (1abk, 10 mg) was dissolved in dichloromethane (0.3 mL), 2 M HCl/ether (0.3 mL) was added dropwise in an ice bath at 0° C. After stirring at room temperature for 3 hours, drying under reduced pressure was performed to obtain the target compound (10.5 mg, ˜97%).

¹H-NMR (300 MHz, CDCl₃) δ 8.35 (br s, 1H), 7.05-7.50 (m, 5H), 3.66 (s, 3H), 3.10-3.30 (m, 4H), 2.40 (s, 3H), 2.00-2.20 (m, 4H).

MS (ESI): m/z=426 [M+H]⁺.

### Example 115

### Preparation of 1-methyl-5-(4-(4-methylpiperazin-1-yl)benzylidene)-2-selenoxo-3-(4-tolyl)-imidazolin-4-one·hydrochloride (1abt·HCl, Compound 115)

After 1-methyl-5-(4-(4-methylpiperazin-1-yl)benzylidene)-2-selenoxo-3-(4-tolyl)-imidazolidin-4-one (1abt, 21 mg) was dissolved in dioxane (0.3 mL), 4 M HCl/dioxane (70 μL) was added dropwise in an ice bath at 0° C. After stirring at room temperature for 3 hours, drying under reduced pressure was performed to obtain the target compound (20 mg, 87%).

¹H-NMR (300 MHz, DMSO-d₆) δ 10.6 (s, 1H), 8.23 (d, J=8.6 Hz, 2H), 7.30 (d, J=8.1 Hz, 2H), 7.23 (d, J=7.9 Hz, 2H), 7.19 (s, 1H), 7.05 (d, J=8.9 Hz, 2H), 3.77 (s, 3H), 2.81 (s, 3H), 2.38 (s, 3H).

MS (EI): m/z=454.1269 [M-HCl]⁺

### Example 116

### Preparation of 1-methyl-5-(4-((2-morpholinoethyl)amino)benzylidene)-2-selenoxo-3-(4-tolyl)-imidazoridin-4-one·hydrochloride (1abw·HCl, Compound 116)

After 1-methyl-5-(4-((2-morpholinoethyl)amino))benzylidene)-2-selenoxo-3-(4-tolyl)-imidazolidin-4-one (1abw, 17 mg) was dissolved in dichloromethane (0.3 mL), 4 M HCl/dioxane (90 μL) was added dropwise in an ice bath at 0° C. After stirring at room temperature for 3 hours, drying under reduced pressure was performed to obtain the target compound (18 mg, ˜96%).

¹H-NMR (300 MHz, DMSO-d₆) δ 11.34 (br s, 1H), 8.19 (d, J=8.7 Hz, 2H), 7.30 (d, J=8.2 Hz, 2H), 7.22 (d, J=8.1 Hz, 2H), 7.15 (s, 1H), 6.71 (d, J=8.8 Hz, 2H), 3.84-4.14 (m, 8H), 3.77 (s, 3H), 3.46-3.65 (m, 4H), 2.38 (s, 3H).

MS (EI): m/z=484.1380 [M-HCl]⁺

### Example 117

### Preparation of 3-(4-ethylphenyl)-1-methyl-5-(4-(piperidin-1-yl)benzylidene)-2-selenoxoimidazolidin-4-one·hydrochloride (1ach·HCl, Compound 117)

After 3-(4-ethylphenyl)-1-methyl-5-(4-(piperidin-1-yl)benzylidene)-2-selenoxoimidazolidin-4-one (1ach, 10 mg) was dissolved in dichloromethane (0.3 mL), 2 M HCl/ether (0.3 mL) was added dropwise in an ice bath at 0° C. After stirring at room temperature for 3 hours, drying under reduced pressure was performed to obtain the target compound (10.3 mg, ˜100%).

¹H-NMR (300 MHz, CDCl₃) δ 8.10 (br s, 1H), 6.90-8.00 (m, 9H), 3.20-4.50 (br s, 5H), 2.69 (br s, 2H), 1.40-1.70 (m, 6H), 1.23 (br s, 3H).

MS (ESI): m/z=454 [M+H-HCl]⁺.

### Example 118

### Preparation of 3-hexyl-1-methyl-5-(4-((2-morpholinoethyl)amino)benzylidene)-2-selenoxoimidazolidin-4-one·hydrochloride (1ajw·HCl, Compound 118)

After 3-hexyl-1-methyl-5-(4-((2-morpholinoethyl)amino)benzylidene)-2-selenoxoimidazolidin-4-one (1ajw, 20 mg) was dissolved in dichloromethane (0.3 mL), 2 M HCl/ether (0.3 mL) was added dropwise in an ice bath at 0° C. After stirring at room temperature for 3 hours, drying under reduced pressure was performed to obtain the target compound (24 mg, ˜100%).

¹H-NMR (300 MHz, CDCl₃) δ 9.37 (br s, 1H), 8.10 (br s, 2H), 6.84 (br s, 2H), 6.65 (s, 1H), 3.95-4.10 (m, 4H), 3.71 (s, 2H), 3.55-3.70 (m, 2H), 3.35-3.53 (m, 4H), 2.85-3.30 (m, 4H), 1.85-1.98 (m, 2H), 1.60-1.82 (m, 2H), 1.26-1.44 (m, 4H), 0.84-0.99 (m, 3H).

MS (EI): m/z=478.1849 [M-HCl]⁺.

### Example 119

### Preparation of 1-methyl-3-phenethyl-5-(4-(pyrrolidin-1-yl)benzylidene)-2-selenoxoimidazolidin-4-one·hydrochloride (1ahk·HCl, Compound 119)

After 1-methyl-3-phenethyl-5-(4-(pyrrolidin-1-yl)benzylidene)-2-selenoxoimidazolidin-4-one (1ahk, 10 mg) was dissolved in dichloromethane (0.5 mL), 2 M HCl/ether (0.3 mL) was added dropwise in an ice bath at 0° C. After stirring at room temperature for 3 hours, drying under reduced pressure was performed to obtain the target compound (10.1 mg, ˜100%).

¹H-NMR (300 MHz, CDCl₃) δ 6.70-8.30 (m, 11H), 3.75-4.80 (br s, 4H), 3.69 (s, 3H), 2.60-3.65 (m, 4H), 1.23 (br s, 4H), 0.82 (br s, 2H).

MS (ESI): m/z=462 [M+Na-HCl]⁺.

### Example 120

### Preparation of 3-cyclopentyl-5-(4-(dimethylamino)benzylidene)-1-methyl-2-selenoxoimidazolidin-4-one·hydrochloride (1akc·HCl, Compound 120)

After 3-cyclopentyl-5-(4-(dimethylamino)benzylidene)-1-methyl-2-selenoxoimidazolidin-4-one (1akc, 10 mg) was dissolved in dichloromethane (0.5 mL), 2 M HCl/ether (0.3 mL) was added dropwise in an ice bath at 0° C. After stirring at room temperature for 3 hours, drying under reduced pressure was performed to obtain the target compound (11 mg, ˜97%).

¹H-NMR (300 MHz, CDCl₃) δ 7.30-8.90 (br s, 5H), 6.91 (br s, 2H), 5.23 (br s, 1H), 2.52-4.10 (m, 9H), 1.80-2.50 (m, 4H), 1.64 (br s, 2H), 1.25 (br s, 2H).

MS (ESI): m/z=378 [M+H-HCl]⁺.

### Example 121

### Preparation of 5-(2,4-dihydroxybenzylidene)-1-methyl-3-(3-morpholinopropyl)-2-selenoxoimidazolidin-4-one·hydrochloride (1aqa·HCl, Compound 121)

After 5-(2,4-dihydroxybenzylidene)-1-methyl-3-(3-morpholinopropyl)-2-selenoxoimidazolidin-4-one (1aqa, 10 mg) was dissolved in dichloromethane (0.5 mL), 4 M HCl/dioxane (59 μL) was added dropwise in an ice bath at 0° C. After stirring at room temperature for 3 hours, drying under reduced pressure was performed to obtain the target compound (2.9 mg, 26%).

¹H-NMR (300 MHz, DMSO-d₆) δ 10.5 (s, 1H), 10.2 (br s, 2H), 8.57 (d, J=8.9 Hz, 0.8H), 8.22 (d, J=8.6 Hz, 0.2H), 7.28 (s, 0.8H), 6.60 (s, 0.2H), 6.42 (d, J=2.3 Hz, 6.37 (d, J=2.3 Hz, 0.2H), 6.29 (dd, J=8.9, 2.3 Hz, 0.8H), 6.20-6.26 (m, 0.2H), 3.89-4.06 (m, 4H), 3.67 (s, 2.4H), 3.56 (s, 0.6H), 2.93-3.21 (m, 8H), 1.93-2.20 (m, 2H).

MS (EI): m/z=425.0855 [M-HCl]⁺.

### Example 122

### Preparation of 5-(4-fluorobenzylidene)-1-methyl-3-(3-morpholinopropyl)-2-selenoxoimidazolidin-4-one·hydrochloride (1aqx·HCl, Compound 122)

After 5-(4-fluorobenzylidene)-1-methyl-3-(3-morpholinopropyl)-2-selenoxoimidazolidin-4-one (1aqx, 14.5 mg) was dissolved in dichloromethane (0.5 mL), 4 M HCl/dioxane (100 μL) was added dropwise in an ice bath at 0° C. After stirring at room temperature for 3 hours, drying under reduced pressure was performed to obtain the target compound (17 mg, 100%).

¹H-NMR (300 MHz, DMSO-d₆) δ 10.7 (br s, 1H), 8.17-8.29 (m, 1.5H), 7.98-8.09 (m, 0.5H), 7.55-7.64 (m, 0.5H), 7.38-7.48 (m, 1.5H), 7.05 (s, 0.7H), 6.57 (s, 0.3H), 3.87-4.05 (m, 2H), 3.71-3.82 (m, 2H), 3.70 (s, 2H), 3.57 (s, 1H), 2.90-3.20 (m, 6H), 1.40-1.76 (m, 4H).

MS (EI): m/z=411.0859 [M-HCl]⁺.

### Experimental Example 1

### Evaluation of Activity to Inhibit Generation of Reactive Oxygen Species and Spore Germination Through NOX Enzyme Inhibition

The inhibition of NADPH oxidase (NOX), which is involved in the generation of reactive oxygen species in red mold, results in the suppression of normal hyphal growth and the formation of abnormal structures known as short-term conidia, thereby inhibiting germination. The abnormal phenotypes caused by the reduction of reactive oxygen species within the red mold can be easily observed through optical microscopy. Through this method, the ability to inhibit the generation of reactive oxygen species by NOX inhibition was evaluated.

Specifically, to evaluate the activity of the compounds, a 0.1 mM concentration of the compound was added to a spore suspension of minimal medium diluted to 1/5 (MM20). Spores of the wild-type strain GZ3639 of red mold (10⁵ spores/ml) were inoculated into the medium and incubated at 25° C. or 24 hours, followed by microscopic observation. Compounds that exhibited more than 50% inhibition of germination and short-term conidia formation compared to normal germination were selected for further activity evaluation at concentrations of 50 μM, 25 μM, and 10 μM. Among these, compounds showing 95% germination inhibition at 10 μM were further evaluated at concentrations of 5 μM, 1 μM, 0.5 μM, and 0.1 μM. Based on these results, the concentration of the compound at which the spore germination inhibition rate reached 50% (inhibitory concentration 50%, IC₅₀) was determined.

The IC₅₀ evaluation results of the compounds of the present disclosure are shown in Table 2 below. However, Examples in Table 2 below show some compounds which are selected and tested among the compounds of the present disclosure, and the present invention is not limited to the compounds listed in Table 2 below, and compounds not listed in Table 2 also exhibit the activity to inhibit generation of reactive oxygen species and spore germination through NOX enzyme inhibition.

TABLE 2

| Compound number | Compound | IC50 (μM) |
|---|---|---|
| 1 | 1aaa | 20 |
| 3 | 1aac | 26 |
| 7 | 1aak | 16 |
| 8 | 1aam | 41 |
| 9 | 1aan | 37 |
| 11 | 1abb | 46 |
| 15 | 1abh | 11 |
| 16 | 1abi | 46 |
| 17 | 1abk | 11 |
| 18 | 1abl | 13 |
| 19 | 1abn | 41 |
| 22 | 1abv | 2.3 |
| 23 | 1abw | 5.2 |
| 24 | 1aca | 14 |
| 27 | 1ace | 9.0 |
| 28 | 1ach | 11 |
| 29 | 1aci | 11 |
| 31 | 1acn | 30 |
| 35 | 1ada | 31 |
| 36 | 1adb | 46 |
| 37 | 1add | 20 |
| 38 | 1ade | 56 |
| 39 | 1adn | 27 |
| 40 | 1aeb | 42 |
| 41 | 1aha | 9.8 |
| 43 | 1ahc | 23 |
| 44 | 1ahe | 1.0 |
| 52 | 1ahs | 3.5 |
| 55 | 1aia | 1.0 |
| 56 | 1aib | 18 |
| 57 | 1aic | 46 |
| 58 | 1aie | 18 |
| 59 | 1aja | 0.68 |
| 60 | 1ajb | 6.0 |
| 61 | 1ajc | 33 |
| 62 | 1aje | 5.0 |
| 63 | 1ajs | 0.50 |
| 67 | 1ajy | 0.45 |
| 68 | 1aka | 3.0 |
| 69 | 1akb | 14 |
| 70 | 1akc | 26 |
| 71 | 1akd | 26 |
| 75 | 1alc | 23 |
| 85 | 1aqi | 5.0 |
| 88 | 1are | 18 |
| 89 | 1asa | 13 |
| 93 | 1aua | 2.9 |
| 95 | 1baa | 22 |
| 96 | 1bad | 44 |
| 101 | 1bci | 45 |
| 102 | 1bck | 40 |
| 104 | 1cab | 23 |
| 106 | 1cad | 34 |
| 107 | 1cae | 23 |
| 108 | 1cca | 22 |
| 109 | 1ccd | 29 |
| 110 | 1cce | 23 |
| 111 | 1cdb | 26 |
| 112 | 1ccd | 31 |

As shown in Table 2, the compounds of the present invention exhibit excellent activity (IC₅₀). In particular, a large number of compounds have an IC₅₀ value of 5 μM or less.

### Experimental Example 2

### Evaluation of Effect of Inhibiting Generation of Reactive Oxygen Species

It is known that the administration of MPP+ or rotenone to cell lines results in the generation of reactive oxygen species (ROS) due to mitochondrial inhibition, the activation of NOX enzymes and the like. The excessively generated ROS can induce various inflammatory responses through multiple pathways. The ROS generated in this process react with the CM-H2DCFDA reagent to produce fluorescence, thereby allowing the indirect quantification of ROS.

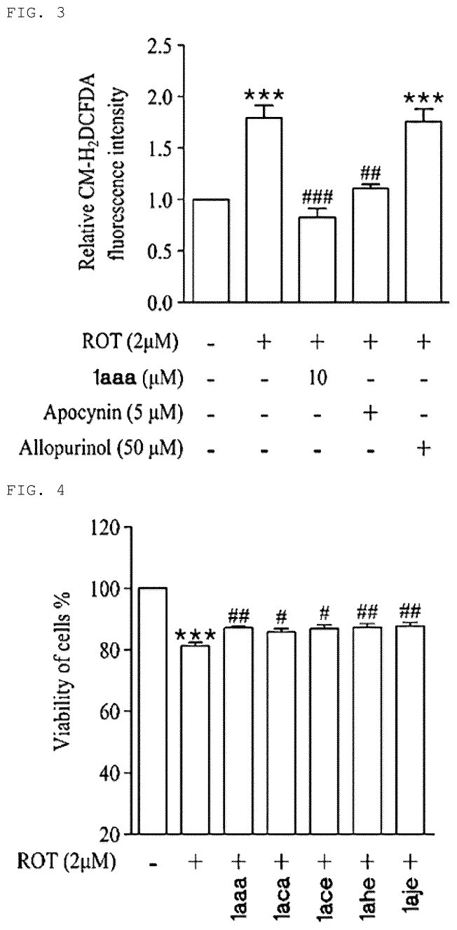

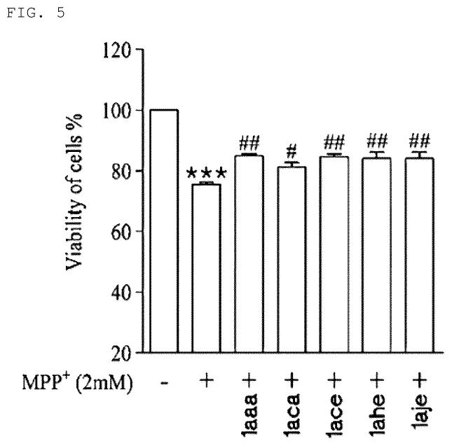

Specifically, the SH-SY5Y cell line was treated with Compound 1aaa (Compound 1, 10 μM), apocynin (5 μM), or allopurinol (50 μM), and then with MPP+ (2 mM) or rotenone (2 μM) for 24 hours. The effect of each compound to inhibit the generation of reactive oxygen species is illustrated in FIGS. 2 and 3 as a relative percentage to that of the control group.

As illustrated in FIGS. 2 and 3, it has been confirmed that Compound 1aaa of the present invention effectively inhibits the increased generation of reactive oxygen species after exposure of the SH-SY5Y cell line to MPP+ or rotenone.

### Experimental Example 3

### Experiment to Evaluate Cell Viability

The treatment of cell lines with the toxic substances MPP+ or rotenone induces cytotoxicity due to various inflammatory and immune response abnormalities. The viability of the cells under these conditions can be measured using the MTT assay.

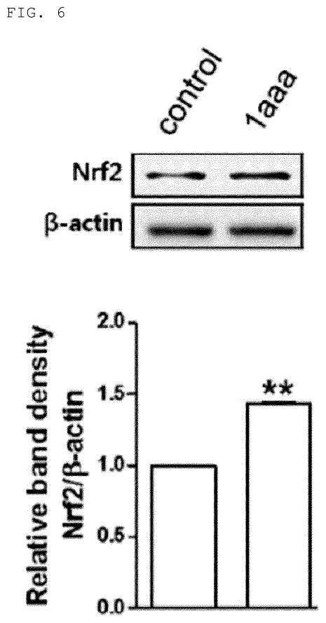

Specifically, the SH-SY5Y cell line was treated with 0.5 μM of Compound 1aaa (Compound 1), 1aca (Compound 24), 1ace (Compound 27), 1ahe (Compound 44) or 1aje (Compound 62) and then with MPP+ (2 mM) or rotenone (2 μM) for 24 hours. Cell viability in the case of being treated with each compound is illustrated in FIGS. 4 and 5.

As illustrated in FIGS. 4 and 5, it has been confirmed that Compounds 1aaa, 1aca, 1ace, 1ahe, and 1aje of the present invention significantly inhibit cell death, which is reduced by MPP+ or rotenone, and improve cell viability.

### Experimental Example 4

### Evaluation of Nrf-2 Induction Effect

When oxidative stress caused by reactive oxygen species increases in cells, the synthesis of antioxidants increases in response to this. One of the transcription factors mediating this antioxidant response is Nrf-2. As the transcription factor Nrf-2 enters the nucleus and the expression of genes related to antioxidation increases, the antioxidant capacity of cells increases,

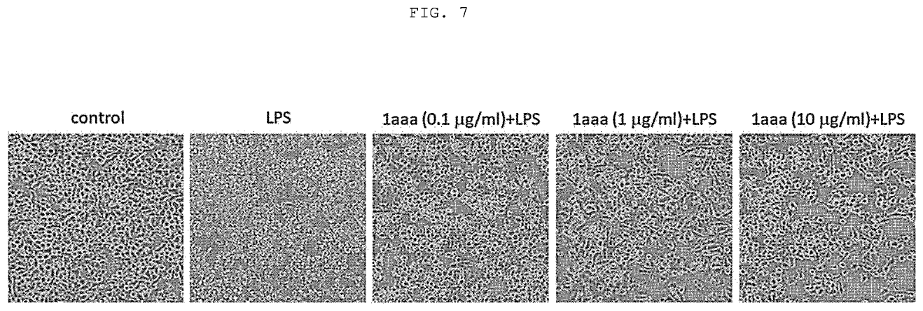

Specifically, the SH-SY5Y cell line was exposed to Compound 1aaa (Compound 1, 10 μM) for 24 hours and then the cells were taken. Thereafter, the nuclear fraction was extracted, then Western blotting was performed to analyze the amount of Nrf-2 in the nucleus, and the results are illustrated in FIG. 6.

As illustrated in FIG. 6, it has been confirmed that the amount of Nrf-2 in the nucleus is increased by about 1.5 times by the treatment with Compound 1aaa of the present invention. Consequently, it can be seen that Compound 1aaa of the present invention activates the cellular antioxidant system by increasing the induction and nuclear import of Nrf-2.

### Experimental Example 5

### Evaluation of Anti-Inflammatory Activity

To evaluate the anti-inflammatory activity of the compounds of the present invention, the morphological changes related to inflammation in cells and the suppression of the expression of inflammatory proteins (IL-1R and COX2) were assessed.

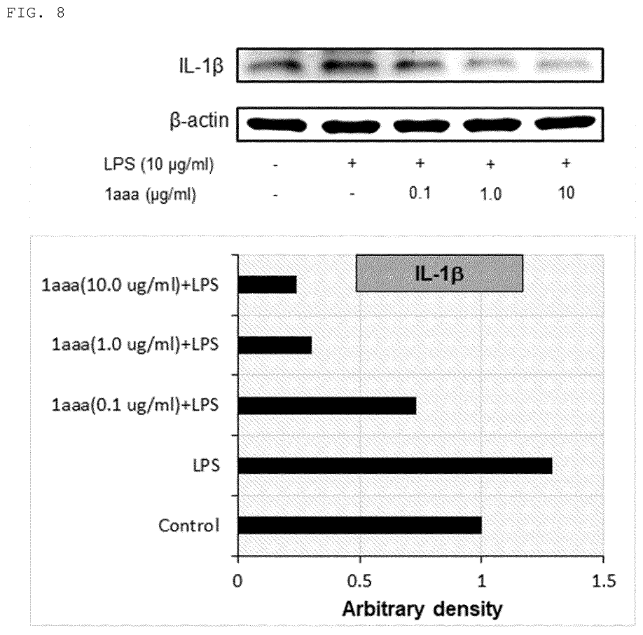

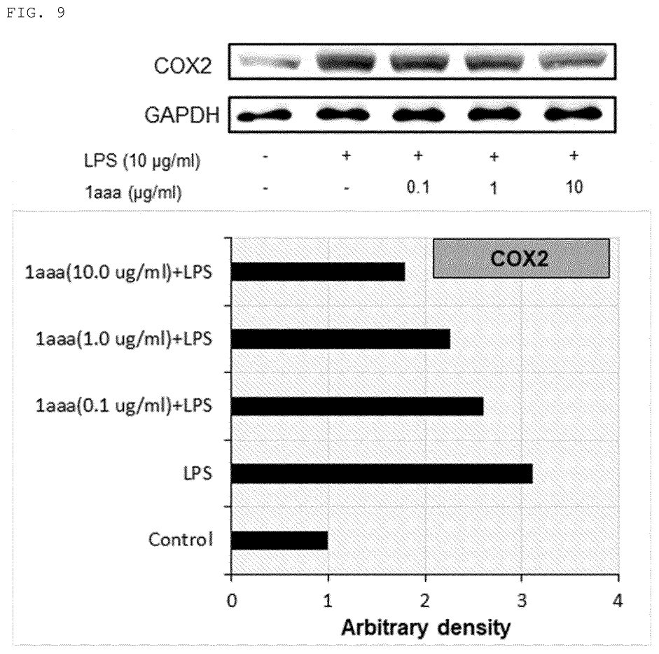

Specifically, BV2 cell lines were treated with compound 1aaa (0.1, 1, 10 μg/ml) for 2 hours, followed by treatment with LPS (10 μg/ml), and then cultured for 24 hours. The morphological changes in the cells treated with the compound were observed, and the results are shown in FIG. 7. Additionally, Western blot analysis was performed to analyze the expression of inflammatory proteins, and the results are shown in FIGS. 8 and 9.

As shown in FIG. 7, LPS stimulated BV cells, inducing the activation of macrophages with a short-branched, rounded morphology, while compound 1aaa of the present invention inhibited the activation of BV2 macrophages in a dose-dependent manner.

Furthermore, as shown in FIGS. 8 and 9, compound 1aaa of the present invention significantly suppressed the expression of IL-1β and COX2.

Therefore, it can be concluded that compound 1aaa of the present invention exhibits anti-inflammatory activity by inhibiting the generation of reactive oxygen species through NOX enzyme inhibition.

### INDUSTRIAL APPLICABILITY

The novel compounds of the present invention, 1-alkyl-5-arylidene-2-selenoxoimidazolidin-4-one or derivatives thereof, inhibit the NOX enzyme that generates reactive oxygen species causing inflammation and regulate Nrf2. Therefore, these compounds and their derivatives, or pharmaceutically acceptable salts thereof, can be usefully employed for the prevention, alleviation, or treatment of inflammatory diseases.

1. A method for the prevention or treatment of inflammatory diseases, comprising administering to a subject a compound of the following chemical formula 1, or a pharmaceutically acceptable salt thereof, as an active ingredient: 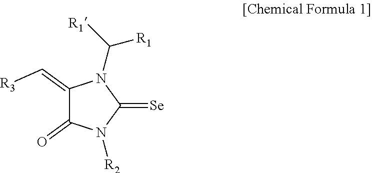
In the above formula,
R₁ and R₁′ are each independently selected from the group consisting of hydrogen, alkyl, cycloalkyl, heterocycloalkyl, aryl, heteroaryl, benzyl, and phenethyl, or R₁ and R₁′ may combine together to form a ring;
R₂ and R₃ are each independently halo, cyano, alkyl, alkoxy, alkenyl, alkynyl, cycloalkyl, heterocycloalkyl, heterocycloalkyl-alkyl, aryl, aryl-alkyl, or heteroaryl;
wherein the alkyl, alkoxy, alkenyl, alkynyl, cycloalkyl, heterocycloalkyl, heterocycloalkyl-alkyl, aryl, aryl-alkyl, or heteroaryl is optionally substituted or unsubstituted with one or more groups selected from hydroxy; halogen; alkyl; -alkyl-hydroxy; -heterocycloalkyl-alkyl-hydroxy; —NH-alkyl-O-alkyl-hydroxy; —NH-alkyl-O-alkyl-halogen; —NH-alkyl-heterocycloalkyl; alkoxy; amino; dialkylamino; nitro; cyano; carbonyl; cycloalkyl; heterocycloalkyl substituted or unsubstituted with alkyl; aryl; and heteroaryl.

2. The method for the prevention or treatment of inflammatory diseases according to claim 1, wherein R₁ and R₁′ are each independently selected from the group consisting of hydrogen, C₁-C₆ alkyl, C₃-C₈ cycloalkyl, C₃-C₈ heterocycloalkyl, C₆-C₁₀ aryl, C₅-C₁₀ heteroaryl, benzyl, and phenethyl, or R₁ and R₁′ may combine together to form a ring.

3. The method for the prevention or treatment of inflammatory diseases according to claim 1, 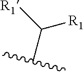
is

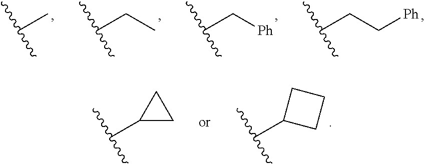

4. The method for the prevention or treatment of inflammatory diseases according to claim 1, wherein R₂ is a C₁-C₁₂ alkyl substituted or unsubstituted with halogen; C₃-C₁₀ cycloalkyl; —C₁-C₆ alkyl-C₃-C₁₀ heterocycloalkyl containing one or more heteroatoms selected from the group consisting of N, S, and O; —C₆-C₁₀ aryl substituted or unsubstituted with halogen or C₁-C₁₂ alkyl; or —C₁-C₆ alkyl-C₆-C₁₀ aryl.

5. The method for the prevention or treatment of inflammatory diseases according to claim 1, wherein R₂ is C₁-C₁₂ alkyl; halogen-substituted C₁-C₆ alkyl; C₃-C₈ cycloalkyl; —C₁-C₆ alkyl-C₃-C₁₀ heterocycloalkyl containing N and O heteroatoms; —C₆-C₁₀ aryl substituted or unsubstituted with halogen or C₁-C₆ alkyl; or —C₁-C₆ alkyl-C₆-C₁₀ aryl.

6. The method for the prevention or treatment of inflammatory diseases according to claim 1, wherein R₂ is C₁-C₁₂ alkyl; C₁-C₆ alkyl substituted with halogen; C₃-C₈ cycloalkyl; —C₁-C₆ alkyl-morpholine; phenyl unsubstituted or substituted with halogen or C₁-C₆ alkyl; or —C₁-C₆ alkyl-phenyl.

7. The method for the prevention or treatment of inflammatory diseases according to claim 1, wherein R₃ is C₆-C₁₀ heterocycloalkyl, C₆-C₁₀ aryl or C₆-C₁₀ heteroaryl, and the heterocycloalkyl, aryl or heteroaryl is substituted or unsubstituted with one or more groups selected from the group consisting of hydroxy; halogen; C₁-C₆ alkyl; —C₁-C₆ alkyl-hydroxy; —C₃-C₈ heterocycloalkyl-C₁-C₆ alkyl-hydroxy; —NHC₁-C₆ alkyl-O—C₁-C₆ alkyl-hydroxy; —NHC₁-C₆ alkyl-O—C₁-C₆ alkyl-halogen; —NHC₁-C₆ alkyl-C₃-C₈ heterocycloalkyl; C₁-C₆ alkoxy; amino; di-C₁-C₆ alkylamino; and C₃-C₈ heterocycloalkyl unsubstituted or substituted with C₁-C₆ alkyl.

8. The method for the prevention or treatment of inflammatory diseases according to claim 1, wherein R₃ is C₆-C₁₀ heterocycloalkyl having a heteroatom N, C₆-C₁₀ aryl or C₆-C₁₀ heteroaryl having a heteroatom N, and the heterocycloalkyl, aryl or heteroaryl is substituted or unsubstituted with one or more groups selected from the group consisting of hydroxy; halogen; C₁-C₆ alkyl; —C₁-C₆ alkyl-hydroxy; —C₃-C₈ heterocycloalkyl-C₁-C₆ alkyl-hydroxy; —NHC₁-C₆ alkyl-O—C₁-C₆ alkyl-hydroxy; —NHC₁-C₆ alkyl-O—C₁-C₆ alkyl-halogen; —NHC₁-C₆ alkyl-C₃-C₈ heterocycloalkyl; C₁-C₆ alkoxy; amino; di-C₁-C₆ alkylamino; and C₃-C₈ heterocycloalkyl unsubstituted or substituted with C₁-C₆ alkyl.

9. The method for the prevention or treatment of inflammatory diseases according to claim 1, wherein R₃ is phenyl, naphthyl, pyridine, piperazine, or imidazole, and the phenyl, naphthyl, pyridine, piperazine, or imidazole is substituted or unsubstituted with one or more groups selected from the group consisting of hydroxy; halogen; C₁-C₆ alkyl; —C₁-C₆ alkyl-hydroxy; -piperazine-C₁-C₆ alkyl-hydroxy; —NHC₁-C₆ alkyl-O—C₁-C₆ alkyl-hydroxy; —NHC₁-C₆ alkyl-O—C₁-C₆ alkyl-halogen; —NHC₁-C₆ alkyl-morpholine; C₁-C₆ alkoxy; amino; di-C₁-C₆ alkylamino; pyrrolidine; piperidine; piperazine unsubstituted or substituted with C₁-C₆ alkyl; and morpholine.

10. The method for the prevention or treatment of inflammatory diseases according to claim 1, wherein the compound is selected from the group consisting of the following compounds:

| Compound | Structural formula |
|---|---|
| 1 | 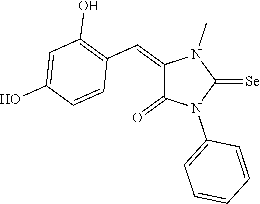 [SMILES: CN1C(=[Se])N(c2ccccc2)C(=O)/C1=C\c1ccc(O)cc1O] |
| 2 | 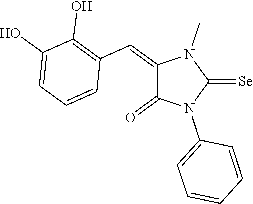 [SMILES: CN1C(=[Se])N(c2ccccc2)C(=O)/C1=C\c1cccc(O)c1O] |
| 3 | 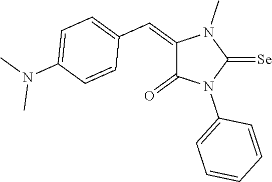 [SMILES: CN1C(=[Se])N(c2ccccc2)C(=O)/C1=C\c1ccc(N(C)C)cc1] |
| 4 | 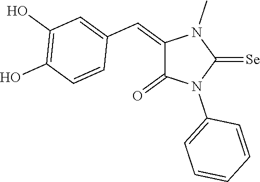 [SMILES: CN1C(=[Se])N(c2ccccc2)C(=O)/C1=C\c1ccc(O)c(O)c1] |
| 5 | 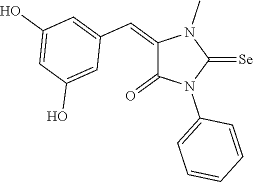 [SMILES: CN1C(=[Se])N(c2ccccc2)C(=O)/C1=C\c1cc(O)cc(O)c1] |
| 6 | 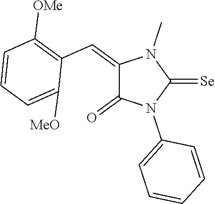 [SMILES: COc1cccc(OC)c1/C=C1\C(=O)N(c2ccccc2)C(=[Se])N1C] |
| 7 | 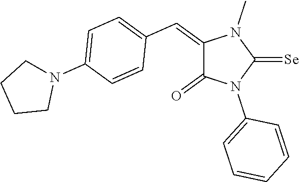 [SMILES: CN1C(=[Se])N(c2ccccc2)C(=O)/C1=C\c1ccc(N2CCCC2)cc1] |
| 8 | 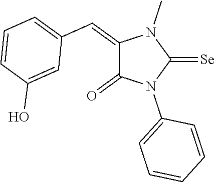 [SMILES: CN1C(=[Se])N(c2ccccc2)C(=O)/C1=C\c1cccc(O)c1] |
| 9 | 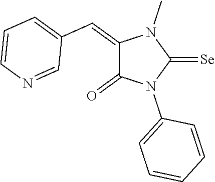 [SMILES: CN1C(=[Se])N(c2ccccc2)C(=O)/C1=C\c1cccnc1] |
| 10 | 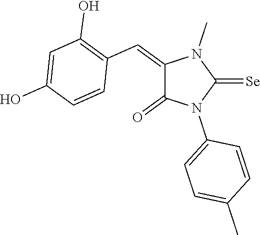 [SMILES: Cc1ccc(N2C(=O)/C(=C\c3ccc(O)cc3O)N(C)C2=[Se])cc1] |
| 11 | 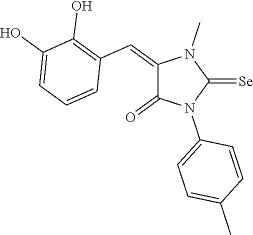 [SMILES: Cc1ccc(N2C(=O)/C(=C\c3cccc(O)c3O)N(C)C2=[Se])cc1] |
| 12 | 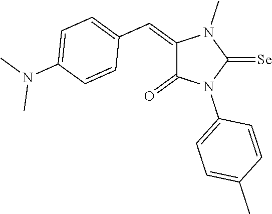 [SMILES: Cc1ccc(N2C(=O)/C(=C\c3ccc(N(C)C)cc3)N(C)C2=[Se])cc1] |
| 13 | 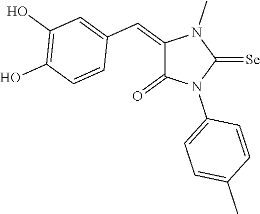 [SMILES: Cc1ccc(N2C(=O)/C(=C\c3ccc(O)c(O)c3)N(C)C2=[Se])cc1] |
| 14 | 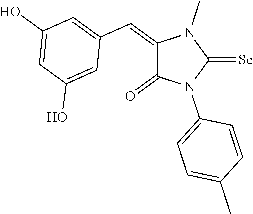 [SMILES: Cc1ccc(N2C(=O)/C(=C\c3cc(O)cc(O)c3)N(C)C2=[Se])cc1] |
| 15 | 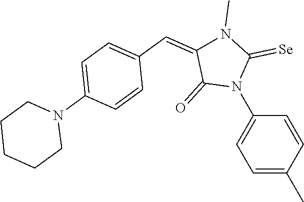 [SMILES: Cc1ccc(N2C(=O)/C(=C\c3ccc(N4CCCCC4)cc3)N(C)C2=[Se])cc1] |
| 16 | 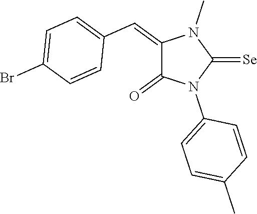 [SMILES: Cc1ccc(N2C(=O)/C(=C\c3ccc(Br)cc3)N(C)C2=[Se])cc1] |
| 17 | 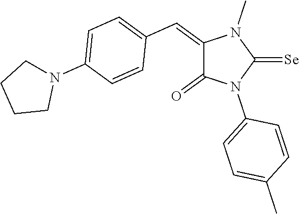 [SMILES: Cc1ccc(N2C(=O)/C(=C\c3ccc(N4CCCC4)cc3)N(C)C2=[Se])cc1] |
| 18 | 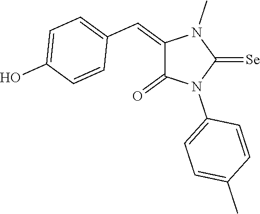 [SMILES: Cc1ccc(N2C(=O)/C(=C\c3ccc(O)cc3)N(C)C2=[Se])cc1] |
| 19 | 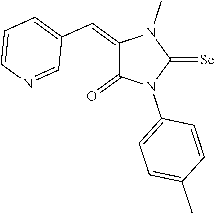 [SMILES: Cc1ccc(N2C(=O)/C(=C\c3cccnc3)N(C)C2=[Se])cc1] |
| 20 | 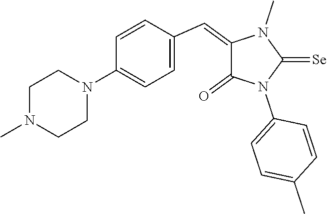 [SMILES: Cc1ccc(N2C(=O)/C(=C\c3ccc(N4CCN(C)CC4)cc3)N(C)C2=[Se])cc1] |
| 21 | 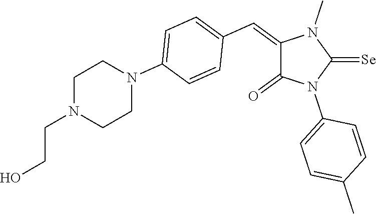 [SMILES: Cc1ccc(N2C(=O)/C(=C\c3ccc(N4CCN(CCO)CC4)cc3)N(C)C2=[Se])cc1] |
| 22 | 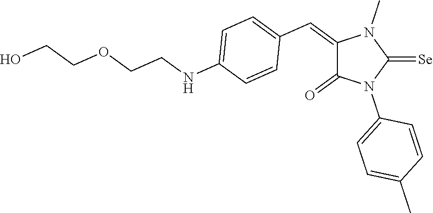 [SMILES: Cc1ccc(N2C(=O)/C(=C\c3ccc(NCCOCCO)cc3)N(C)C2=[Se])cc1] |
| 23 | 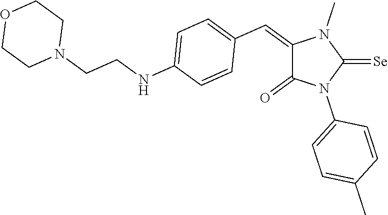 [SMILES: Cc1ccc(N2C(=O)/C(=C\c3ccc(NCCN4CCOCC4)cc3)N(C)C2=[Se])cc1] |
| 24 | 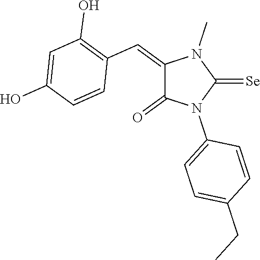 [SMILES: CCc1ccc(N2C(=O)/C(=C\c3ccc(O)cc3O)N(C)C2=[Se])cc1] |
| 25 | 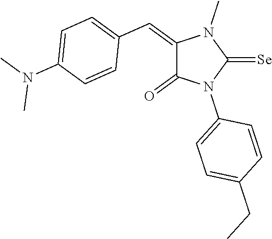 [SMILES: CCc1ccc(N2C(=O)/C(=C\c3ccc(N(C)C)cc3)N(C)C2=[Se])cc1] |
| 26 | 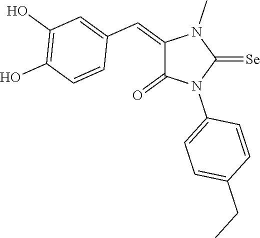 [SMILES: CCc1ccc(N2C(=O)/C(=C\c3ccc(O)c(O)c3)N(C)C2=[Se])cc1] |
| 27 | 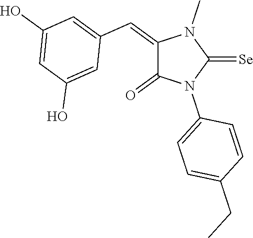 [SMILES: CCc1ccc(N2C(=O)/C(=C\c3cc(O)cc(O)c3)N(C)C2=[Se])cc1] |
| 28 | 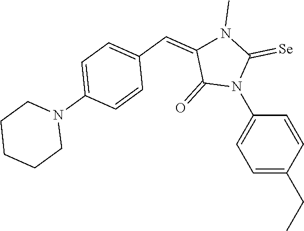 [SMILES: CCc1ccc(N2C(=O)/C(=C\c3ccc(N4CCCCC4)cc3)N(C)C2=[Se])cc1] |
| 29 | 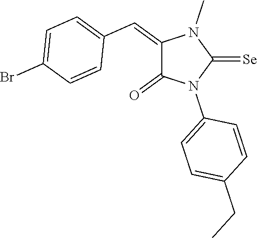 [SMILES: CCc1ccc(N2C(=O)/C(=C\c3ccc(Br)cc3)N(C)C2=[Se])cc1] |
| 30 | 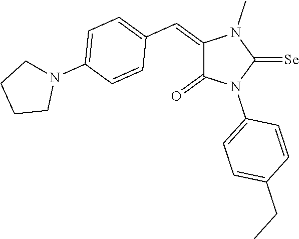 [SMILES: CCc1ccc(N2C(=O)/C(=C\c3ccc(N4CCCC4)cc3)N(C)C2=[Se])cc1] |
| 31 | 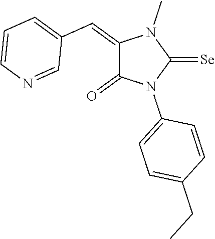 [SMILES: CCc1ccc(N2C(=O)/C(=C\c3cccnc3)N(C)C2=[Se])cc1] |
| 32 | 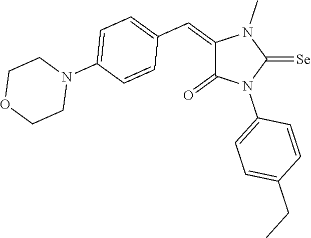 [SMILES: CCc1ccc(N2C(=O)/C(=C\c3ccc(N4CCOCC4)cc3)N(C)C2=[Se])cc1] |
| 33 | 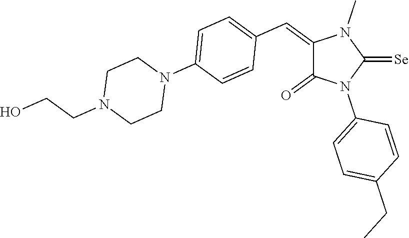 [SMILES: CCc1ccc(N2C(=O)/C(=C\c3ccc(N4CCN(CCO)CC4)cc3)N(C)C2=[Se])cc1] |
| 34 | 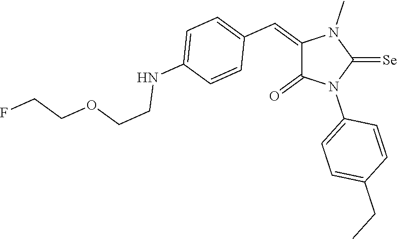 [SMILES: CCc1ccc(N2C(=O)/C(=C\c3ccc(NCCOCCF)cc3)N(C)C2=[Se])cc1] |
| 35 | 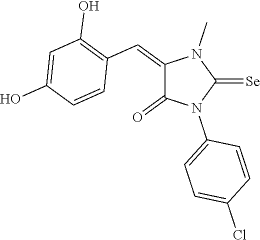 [SMILES: CN1C(=[Se])N(c2ccc(Cl)cc2)C(=O)/C1=C\c1ccc(O)cc1O] |
| 36 | 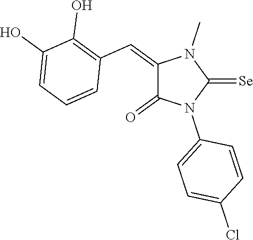 [SMILES: CN1C(=[Se])N(c2ccc(Cl)cc2)C(=O)/C1=C\c1cccc(O)c1O] |
| 37 | 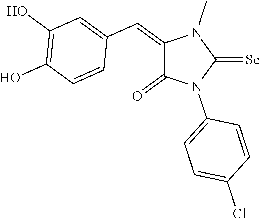 [SMILES: CN1C(=[Se])N(c2ccc(Cl)cc2)C(=O)/C1=C\c1ccc(O)c(O)c1] |
| 38 | 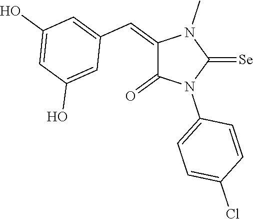 [SMILES: CN1C(=[Se])N(c2ccc(Cl)cc2)C(=O)/C1=C\c1cc(O)cc(O)c1] |
| 39 | 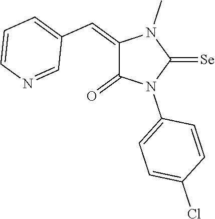 [SMILES: CN1C(=[Se])N(c2ccc(Cl)cc2)C(=O)/C1=C\c1cccnc1] |
| 40 | 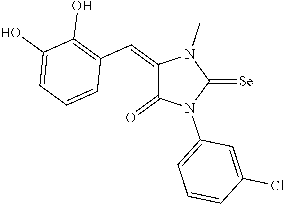 [SMILES: CN1C(=[Se])N(c2cccc(Cl)c2)C(=O)/C1=C\c1cccc(O)c1O] |
| 41 | 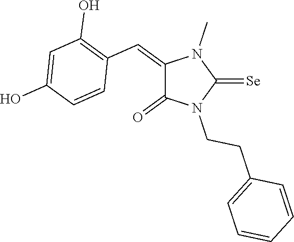 [SMILES: CN1C(=[Se])N(CCc2ccccc2)C(=O)/C1=C\c1ccc(O)cc1O] |
| 42 | 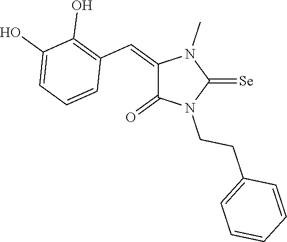 [SMILES: CN1C(=[Se])N(CCc2ccccc2)C(=O)/C1=C\c1cccc(O)c1O] |
| 43 | 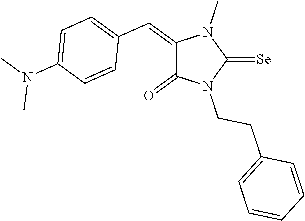 [SMILES: CN1C(=[Se])N(CCc2ccccc2)C(=O)/C1=C\c1ccc(N(C)C)cc1] |
| 44 | 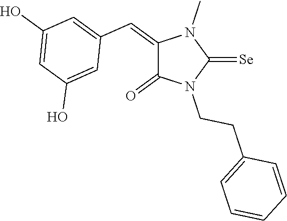 [SMILES: CN1C(=[Se])N(CCc2ccccc2)C(=O)/C1=C\c1cc(O)cc(O)c1] |
| 45 | 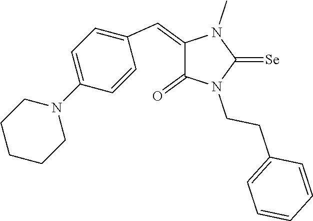 [SMILES: CN1C(=[Se])N(CCc2ccccc2)C(=O)/C1=C\c1ccc(N2CCCCC2)cc1] |
| 46 | 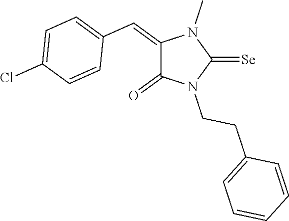 [SMILES: CN1C(=[Se])N(CCc2ccccc2)C(=O)/C1=C\c1ccc(Cl)cc1] |
| 47 | 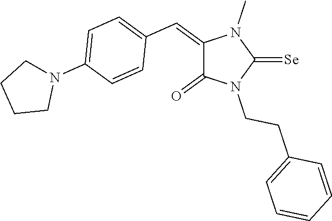 [SMILES: CN1C(=[Se])N(CCc2ccccc2)C(=O)/C1=C\c1ccc(N2CCCC2)cc1] |
| 48 | 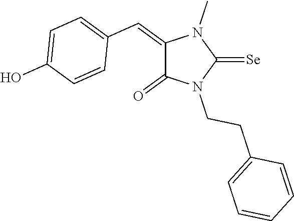 [SMILES: CN1C(=[Se])N(CCc2ccccc2)C(=O)/C1=C\c1ccc(O)cc1] |
| 49 | 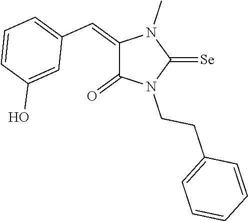 [SMILES: CN1C(=[Se])N(CCc2ccccc2)C(=O)/C1=C\c1cccc(O)c1] |
| 50 | 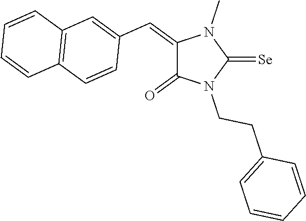 [SMILES: CN1C(=[Se])N(CCc2ccccc2)C(=O)/C1=C\c1ccc2ccccc2c1] |
| 51 | 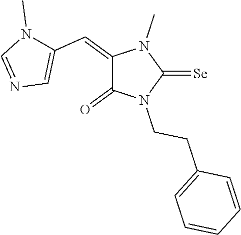 [SMILES: CN1C(=[Se])N(CCc2ccccc2)C(=O)/C1=C\c1cncn1C] |
| 52 | 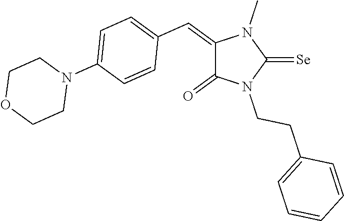 [SMILES: CN1C(=[Se])N(CCc2ccccc2)C(=O)/C1=C\c1ccc(N2CCOCC2)cc1] |
| 53 | 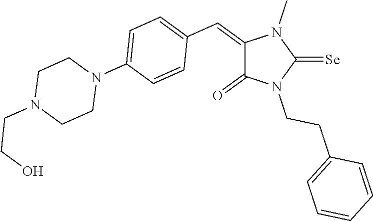 [SMILES: CN1C(=[Se])N(CCc2ccccc2)C(=O)/C1=C\c1ccc(N2CCN(CCO)CC2)cc1] |
| 54 | 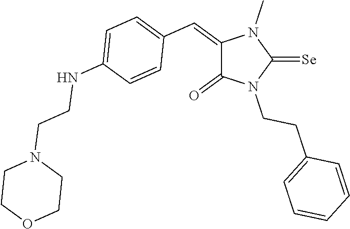 [SMILES: CN1C(=[Se])N(CCc2ccccc2)C(=O)/C1=C\c1ccc(NCCN2CCOCC2)cc1] |
| 55 | 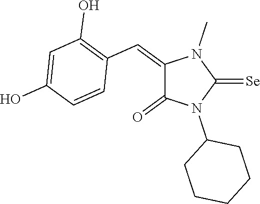 [SMILES: CN1C(=[Se])N(C2CCCCC2)C(=O)/C1=C\c1ccc(O)cc1O] |
| 56 | 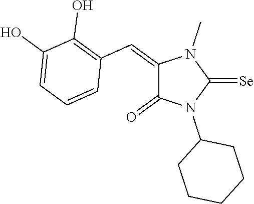 [SMILES: CN1C(=[Se])N(C2CCCCC2)C(=O)/C1=C\c1cccc(O)c1O] |
| 57 | 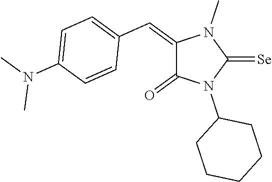 [SMILES: CN1C(=[Se])N(C2CCCCC2)C(=O)/C1=C\c1ccc(N(C)C)cc1] |
| 58 | 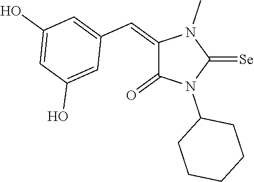 [SMILES: CN1C(=[Se])N(C2CCCCC2)C(=O)/C1=C\c1cc(O)cc(O)c1] |
| 59 | 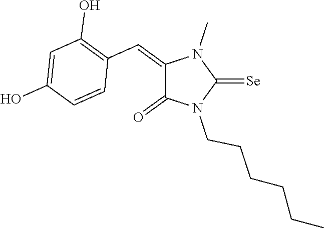 [SMILES: CCCCCCN1C(=O)/C(=C\c2ccc(O)cc2O)N(C)C1=[Se]] |
| 60 | 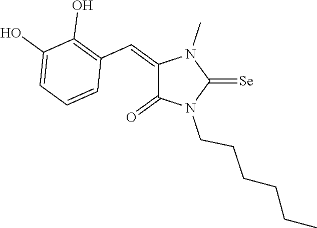 [SMILES: CCCCCCN1C(=O)/C(=C\c2cccc(O)c2O)N(C)C1=[Se]] |
| 61 | 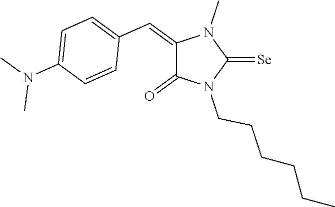 [SMILES: CCCCCCN1C(=O)/C(=C\c2ccc(N(C)C)cc2)N(C)C1=[Se]] |
| 62 | 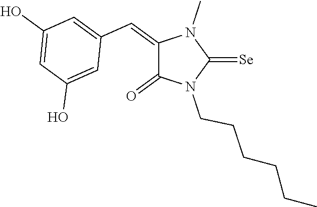 [SMILES: CCCCCCN1C(=O)/C(=C\c2cc(O)cc(O)c2)N(C)C1=[Se]] |
| 63 | 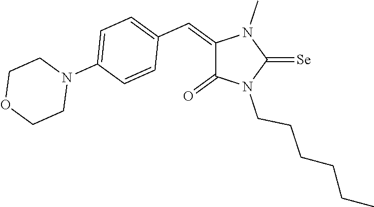 [SMILES: CCCCCCN1C(=O)/C(=C\c2ccc(N3CCOCC3)cc2)N(C)C1=[Se]] |
| 64 | 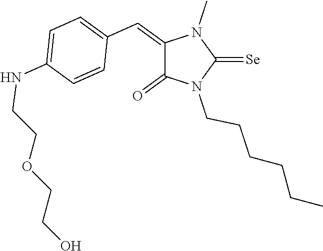 [SMILES: CCCCCCN1C(=O)/C(=C\c2ccc(NCCOCCO)cc2)N(C)C1=[Se]] |
| 65 | 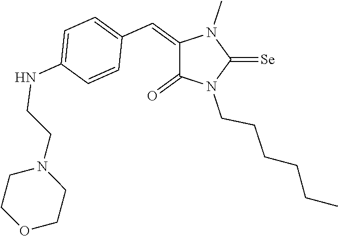 [SMILES: CCCCCCN1C(=O)/C(=C\c2ccc(NCCN3CCOCC3)cc2)N(C)C1=[Se]] |
| 66 | 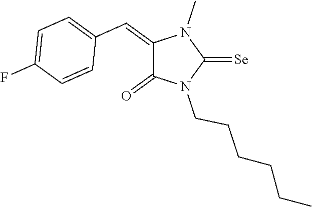 [SMILES: CCCCCCN1C(=O)/C(=C\c2ccc(F)cc2)N(C)C1=[Se]] |
| 67 | 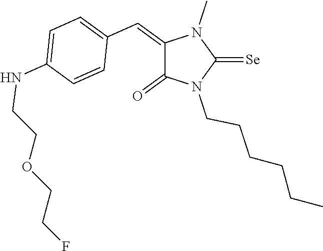 [SMILES: CCCCCCN1C(=O)/C(=C\c2ccc(NCCOCCF)cc2)N(C)C1=[Se]] |
| 68 | 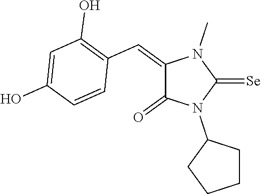 [SMILES: CN1C(=[Se])N(C2CCCC2)C(=O)/C1=C\c1ccc(O)cc1O] |
| 69 | 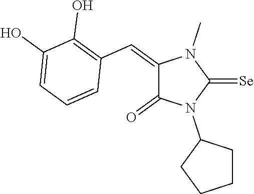 [SMILES: CN1C(=[Se])N(C2CCCC2)C(=O)/C1=C\c1cccc(O)c1O] |
| 70 | 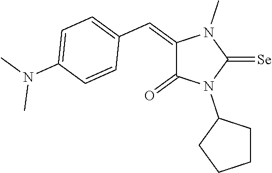 [SMILES: CN1C(=[Se])N(C2CCCC2)C(=O)/C1=C\c1ccc(N(C)C)cc1] |
| 71 | 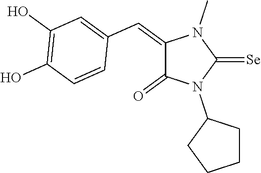 [SMILES: CN1C(=[Se])N(C2CCCC2)C(=O)/C1=C\c1ccc(O)c(O)c1] |
| 72 | 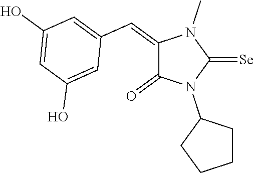 [SMILES: CN1C(=[Se])N(C2CCCC2)C(=O)/C1=C\c1cc(O)cc(O)c1] |
| 73 | 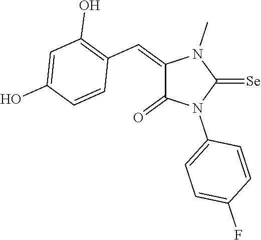 [SMILES: CN1C(=[Se])N(c2ccc(F)cc2)C(=O)/C1=C\c1ccc(O)cc1O] |
| 74 | 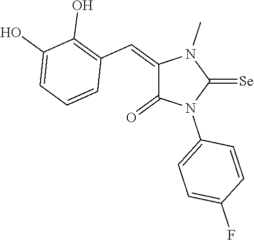 [SMILES: CN1C(=[Se])N(c2ccc(F)cc2)C(=O)/C1=C\c1cccc(O)c1O] |
| 75 | 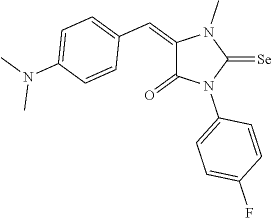 [SMILES: CN1C(=[Se])N(c2ccc(F)cc2)C(=O)/C1=C\c1ccc(N(C)C)cc1] |
| 76 | 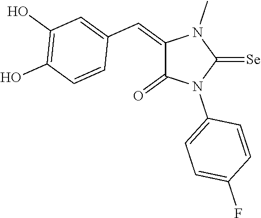 [SMILES: CN1C(=[Se])N(c2ccc(F)cc2)C(=O)/C1=C\c1ccc(O)c(O)c1] |
| 77 | 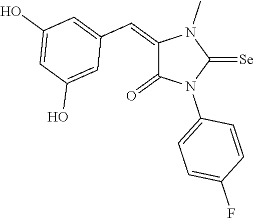 [SMILES: CN1C(=[Se])N(c2ccc(F)cc2)C(=O)/C1=C\c1cc(O)cc(O)c1] |
| 78 | 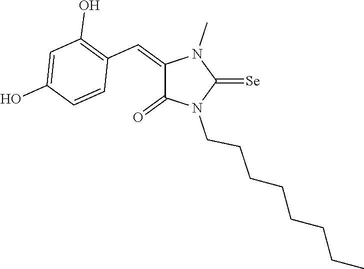 [SMILES: CCCCCCCCN1C(=O)/C(=C\c2ccc(O)cc2O)N(C)C1=[Se]] |
| 79 | 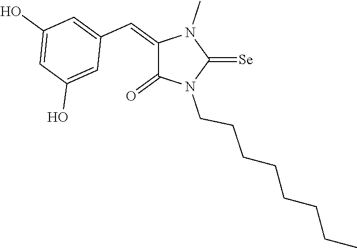 [SMILES: CCCCCCCCN1C(=O)/C(=C\c2cc(O)cc(O)c2)N(C)C1=[Se]] |
| 80 | 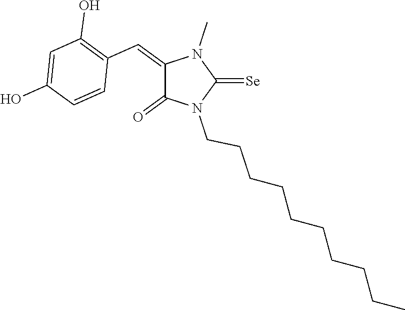 [SMILES: CCCCCCCCCCN1C(=O)/C(=C\c2ccc(O)cc2O)N(C)C1=[Se]] |
| 81 | 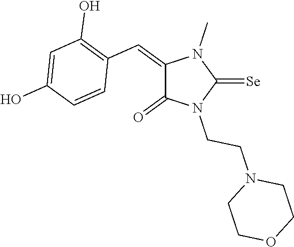 [SMILES: CN1C(=[Se])N(CCN2CCOCC2)C(=O)/C1=C\c1ccc(O)cc1O] |
| 82 | 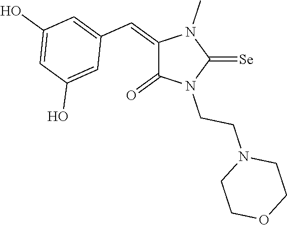 [SMILES: CN1C(=[Se])N(CCN2CCOCC2)C(=O)/C1=C\c1cc(O)cc(O)c1] |
| 83 | 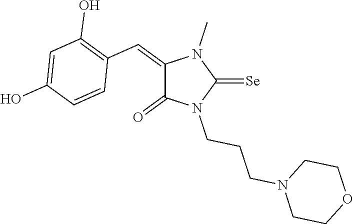 [SMILES: CN1C(=[Se])N(CCCN2CCOCC2)C(=O)/C1=C\c1ccc(O)cc1O] |
| 84 | 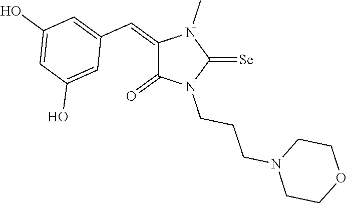 [SMILES: CN1C(=[Se])N(CCCN2CCOCC2)C(=O)/C1=C\c1cc(O)cc(O)c1] |
| 85 | 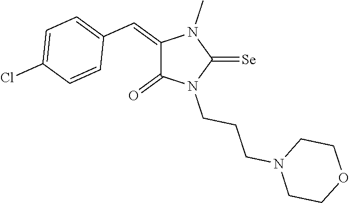 [SMILES: CN1C(=[Se])N(CCCN2CCOCC2)C(=O)/C1=C\c1ccc(Cl)cc1] |
| 86 | 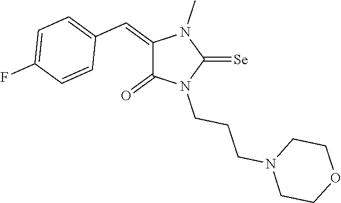 [SMILES: CN1C(=[Se])N(CCCN2CCOCC2)C(=O)/C1=C\c1ccc(F)cc1] |
| 87 | 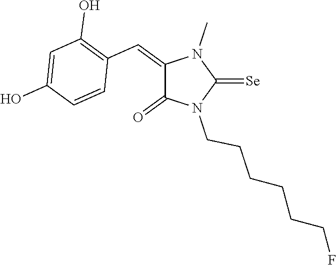 [SMILES: CN1C(=[Se])N(CCCCCCF)C(=O)/C1=C\c1ccc(O)cc1O] |
| 88 | 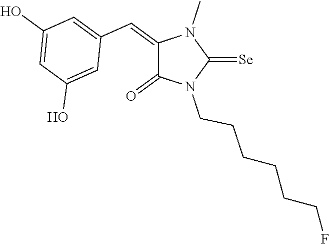 [SMILES: CN1C(=[Se])N(CCCCCCF)C(=O)/C1=C\c1cc(O)cc(O)c1] |
| 89 | 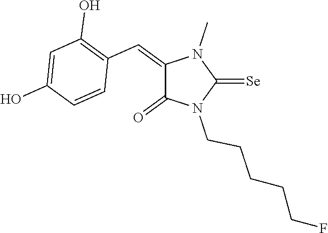 [SMILES: CN1C(=[Se])N(CCCCCF)C(=O)/C1=C\c1ccc(O)cc1O] |
| 90 | 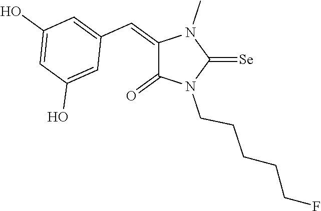 [SMILES: CN1C(=[Se])N(CCCCCF)C(=O)/C1=C\c1cc(O)cc(O)c1] |
| 91 | 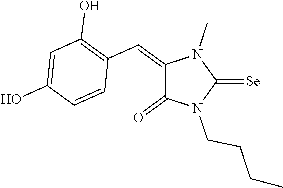 [SMILES: CCCCN1C(=O)/C(=C\c2ccc(O)cc2O)N(C)C1=[Se]] |
| 92 | 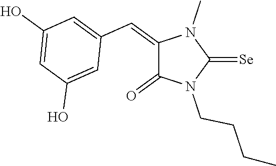 [SMILES: CCCCN1C(=O)/C(=C\c2cc(O)cc(O)c2)N(C)C1=[Se]] |
| 93 | 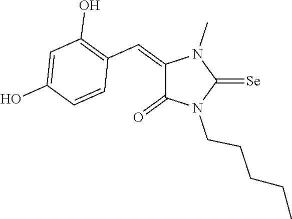 [SMILES: CCCCCN1C(=O)/C(=C\c2ccc(O)cc2O)N(C)C1=[Se]] |
| 94 | 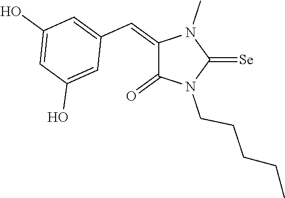 [SMILES: CCCCCN1C(=O)/C(=C\c2cc(O)cc(O)c2)N(C)C1=[Se]] |
| 95 | 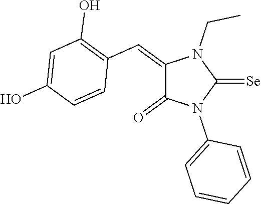 [SMILES: CCN1C(=[Se])N(c2ccccc2)C(=O)/C1=C\c1ccc(O)cc1O] |
| 96 | 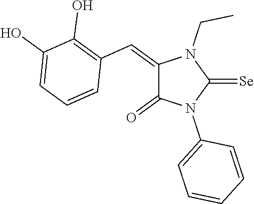 [SMILES: CCN1C(=[Se])N(c2ccccc2)C(=O)/C1=C\c1cccc(O)c1O] |
| 97 | 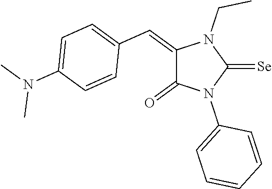 [SMILES: CCN1C(=[Se])N(c2ccccc2)C(=O)/C1=C\c1ccc(N(C)C)cc1] |
| 98 | 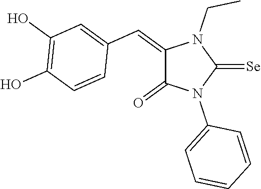 [SMILES: CCN1C(=[Se])N(c2ccccc2)C(=O)/C1=C\c1ccc(O)c(O)c1] |
| 99 | 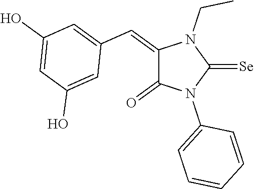 [SMILES: CCN1C(=[Se])N(c2ccccc2)C(=O)/C1=C\c1cc(O)cc(O)c1] |
| 100 | 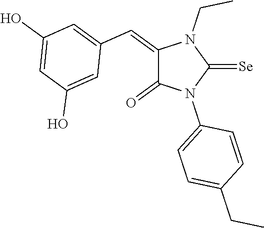 [SMILES: CCc1ccc(N2C(=O)/C(=C\c3cc(O)cc(O)c3)N(CC)C2=[Se])cc1] |
| 101 | 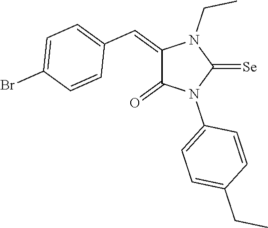 [SMILES: CCc1ccc(N2C(=O)/C(=C\c3ccc(Br)cc3)N(CC)C2=[Se])cc1] |
| 102 | 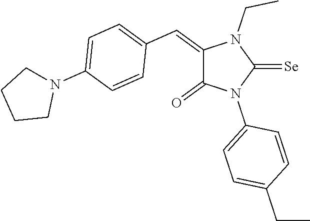 [SMILES: CCc1ccc(N2C(=O)/C(=C\c3ccc(N4CCCC4)cc3)N(CC)C2=[Se])cc1] |
| 103 | 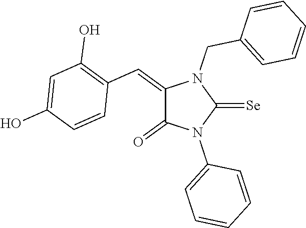 [SMILES: O=C1/C(=C\c2ccc(O)cc2O)N(Cc2ccccc2)C(=[Se])N1c1ccccc1] |
| 104 | 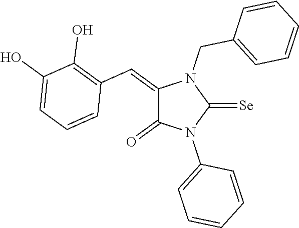 [SMILES: O=C1/C(=C\c2cccc(O)c2O)N(Cc2ccccc2)C(=[Se])N1c1ccccc1] |
| 105 | 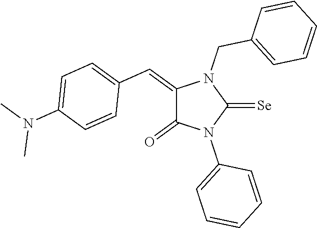 [SMILES: CN(C)c1ccc(/C=C2\C(=O)N(c3ccccc3)C(=[Se])N2Cc2ccccc2)cc1] |
| 106 | 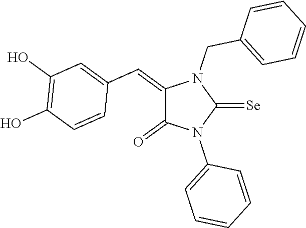 [SMILES: O=C1/C(=C\c2ccc(O)c(O)c2)N(Cc2ccccc2)C(=[Se])N1c1ccccc1] |
| 107 | 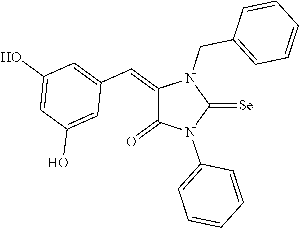 [SMILES: O=C1/C(=C\c2cc(O)cc(O)c2)N(Cc2ccccc2)C(=[Se])N1c1ccccc1] |
| 108 | 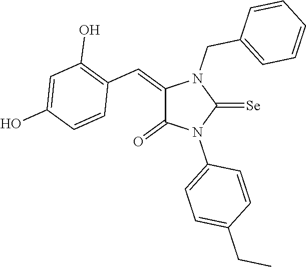 [SMILES: CCc1ccc(N2C(=O)/C(=C\c3ccc(O)cc3O)N(Cc3ccccc3)C2=[Se])cc1] |
| 109 | 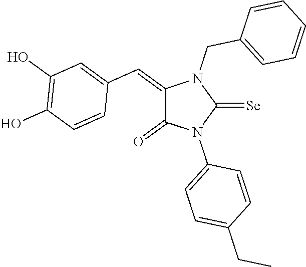 [SMILES: CCc1ccc(N2C(=O)/C(=C\c3ccc(O)c(O)c3)N(Cc3ccccc3)C2=[Se])cc1] |
| 110 | 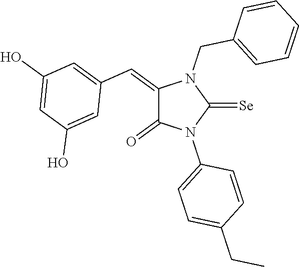 [SMILES: CCc1ccc(N2C(=O)/C(=C\c3cc(O)cc(O)c3)N(Cc3ccccc3)C2=[Se])cc1] |
| 111 | 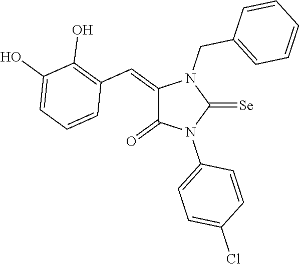 [SMILES: O=C1/C(=C\c2cccc(O)c2O)N(Cc2ccccc2)C(=[Se])N1c1ccc(Cl)cc1] |
| 112 | 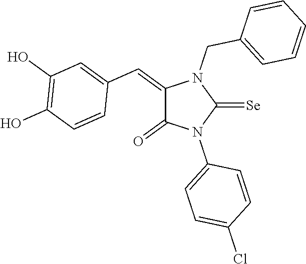 [SMILES: O=C1/C(=C\c2ccc(O)c(O)c2)N(Cc2ccccc2)C(=[Se])N1c1ccc(Cl)cc1] |
| 113 | 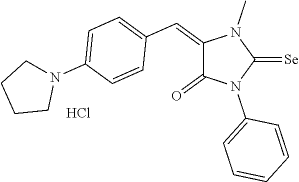 [SMILES: CN1C(=[Se])N(c2ccccc2)C(=O)/C1=C\c1ccc(N2CCCC2)cc1 ; Cl] |
| 114 | 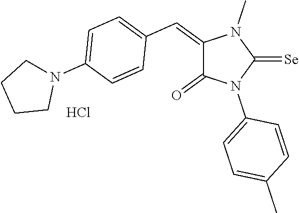 [SMILES: Cc1ccc(N2C(=O)/C(=C\c3ccc(N4CCCC4)cc3)N(C)C2=[Se])cc1 ; Cl] |
| 115 | 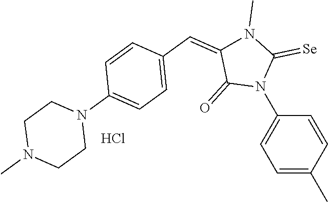 |
| 116 | 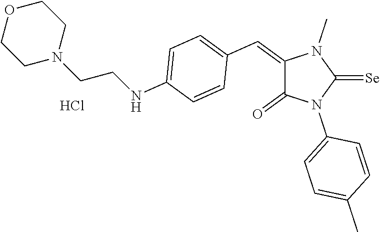 [SMILES: Cc1ccc(N2C(=O)/C(=C\c3ccc(NCCN4CCOCC4)cc3)N(C)C2=[Se])cc1 ; Cl] |
| 117 | 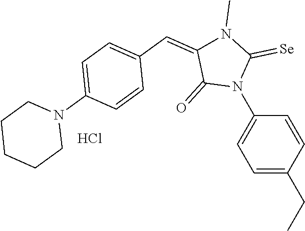 [SMILES: CCc1ccc(N2C(=O)/C(=C\c3ccc(N4CCCCC4)cc3)N(C)C2=[Se])cc1 ; Cl] |
| 118 | 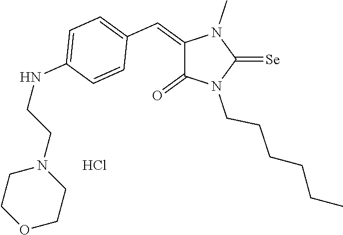 [SMILES: CCCCCCN1C(=O)/C(=C\c2ccc(NCCN3CCOCC3)cc2)N(C)C1=[Se] ; Cl] |
| 119 | 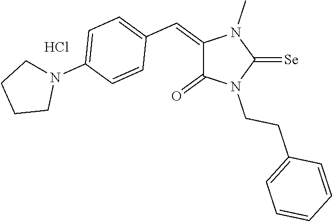 [SMILES: CN1C(=[Se])N(CCc2ccccc2)C(=O)/C1=C\c1ccc(N2CCCC2)cc1 ; Cl] |
| 120 | 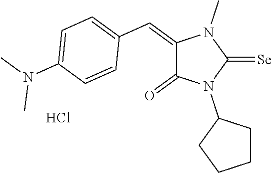 [SMILES: CN1C(=[Se])N(C2CCCC2)C(=O)/C1=C\c1ccc(N(C)C)cc1 ; Cl] |
| 121 | 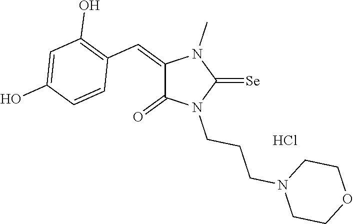 [SMILES: CN1C(=[Se])N(CCCN2CCOCC2)C(=O)/C1=C\c1ccc(O)cc1O ; Cl] |
| 122 | 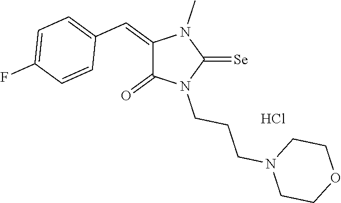 [SMILES: CN1C(=[Se])N(CCCN2CCOCC2)C(=O)/C1=C\c1ccc(F)cc1 ; Cl] |

11. The method for the prevention or treatment of inflammatory diseases according to claim 1, wherein the compound of chemical formula 1 is prepared through the following steps:
1) reacting an amine compound of Chemical Formula 5 with formic acid to produce a formamide compound of Chemical Formula 6;
2) reacting the formamide compound with selenium (Se) to produce an isoselenocyanate compound of Formula 7 under conditions comprising one or more selected from triphosgene, dichloromethane, and triethylamine;
3) reacting the isoselenocyanate compound in a solvent with an amino acid compound of Chemical Formula 8 to produce a 1-alkyl-2-selenoxoimidazolidin-4-one compound of Chemical Formula 9; and
4) reacting the 1-alkyl-2-selenoxoimidazolidin-4-one compound with an aldehyde compound of Chemical Formula 10 to produce the compound of Formula 1; 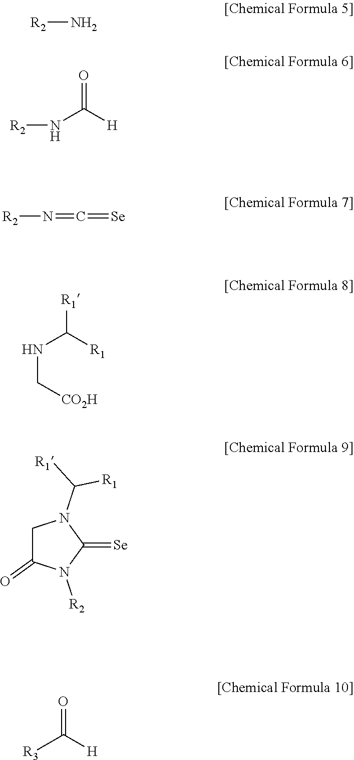
where,
R₁ and R₁′ are each independently selected from the group consisting of hydrogen, alkyl, cycloalkyl, heterocycloalkyl, aryl, heteroaryl, benzyl, and a phenethyl, or R₁ and R₁′ may combine together to form a ring;
R₂ and R₃ are each independently halo, cyano, alkyl, alkoxy, alkenyl, alkynyl, cycloalkyl, heterocycloalkyl, heterocycloalkyl-alkyl, aryl, aryl-alkyl, or heteroaryl;
and the alkyl, alkoxy, alkenyl, alkynyl, cycloalkyl, heterocycloalkyl, heterocycloalkyl-alkyl, aryl, aryl-alkyl, or heteroaryl is optionally substituted or unsubstituted with one or more groups selected from the group consisting of hydroxy; halogen; alkyl; -alkyl-hydroxy; -heterocycloalkyl-alkyl-hydroxy; —NH alkyl-O-alkyl-hydroxy; —NH alkyl-O-alkyl-halogen; —NH alkyl-heterocycloalkyl; alkoxy; amino; dialkylamino; nitro; cyano; carbonyl; cycloalkyl; heterocycloalkyl unsubstituted or substituted with alkyl; aryl; and heteroaryl.

12. The method for the prevention or treatment of inflammatory diseases according to claim 11, wherein 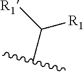
is

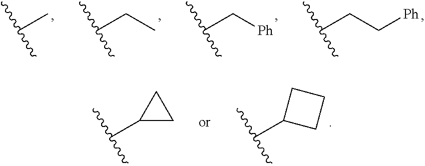

13. The method for the prevention or treatment of inflammatory diseases according to claim 11, wherein R₂ is C₁-C₁₂ alkyl; C₁-C₆ alkyl substituted with halogen; C₃-C₈ cycloalkyl; —C₁-C₆ alkyl-morpholine; phenyl unsubstituted or substituted with halogen or C₁-C₆ alkyl; or —C₁-C₆ alkyl-phenyl.

14. The method for the prevention or treatment of inflammatory diseases according to claim 11, wherein R₃ is phenyl, naphthyl, pyridine, piperazine, or imidazole, and the phenyl, naphthyl, pyridine, piperazine, or imidazole is substituted or unsubstituted with one or more groups selected from the group consisting of hydroxy; halogen; C₁-C₆ alkyl; —C₁-C₆ alkyl-hydroxy; -piperazine-C₁-C₆ alkyl-hydroxy; —NHC₁-C₆ alkyl-O—C₁-C₆ alkyl-hydroxy; —NHC₁-C₆ alkyl-O—C₁-C₆ alkyl-halogen; —NHC₁-C₆ alkyl-morpholine; C₁-C₆ alkoxy; amino; di-C₁-C₆ alkylamino; pyrrolidine; piperidine; piperazine unsubstituted or substituted with C₁-C₆ alkyl; and morpholine.

15. The method for the prevention or treatment of inflammatory diseases according to claim 11, wherein the solvent is one or more selected from the group consisting of dioxane, methanol, ethanol, acetonitrile, tetrahydrofuran (THF), dimethylformamide (DMF), dimethyl sulfoxide (DMSO), and dichloroethylene (DCE).

16. The method for the prevention or treatment of inflammatory diseases according to claim 1, wherein the inflammatory diseases may be one or more selected from the group consisting of sepsis, septic shock, inflammatory bowel disease (IBD), peritonitis, nephritis, diabetic nephropathy, diabetic retinopathy, acute bronchitis, chronic bronchitis, osteoarthritis, ankylosing spondylitis, chronic obstructive pulmonary disease (COPD), rheumatoid arthritis, acute lung injury, and bronchopulmonary dysplasia.

17. The method for the prevention or treatment of inflammatory diseases according to claim 16, wherein the inflammatory bowel disease is ulcerative colitis (UC) or Crohn's disease.

18. A method for the improvement of inflammatory diseases comprising administering to a subject a compound of the following Chemical Formula 1 or a pharmaceutically acceptable salt thereof, as an active ingredient: 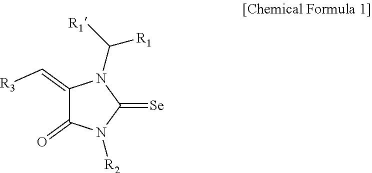
in the above formula,
R₁ and R₁′ are each independently selected from the group consisting of hydrogen, alkyl, cycloalkyl, heterocycloalkyl, aryl, heteroaryl, benzyl, and phenethyl, or R₁ and R₁′ may combine together to form a ring;
R₂ and R₃ are each independently halo, cyano, alkyl, alkoxy, alkenyl, alkynyl, cycloalkyl, heterocycloalkyl, heterocycloalkyl-alkyl, aryl, aryl-alkyl, or heteroaryl;
wherein the alkyl, alkoxy, alkenyl, alkynyl, cycloalkyl, heterocycloalkyl, heterocycloalkyl-alkyl, aryl, aryl-alkyl, or heteroaryl is optionally substituted or unsubstituted with one or more groups selected from hydroxy; halogen; alkyl; -alkyl-hydroxy; -heterocycloalkyl-alkyl-hydroxy; —NH-alkyl-O-alkyl-hydroxy; —NH-alkyl-O-alkyl-halogen; —NH-alkyl-heterocycloalkyl; alkoxy; amino; dialkylamino; nitro; cyano; carbonyl; cycloalkyl; heterocycloalkyl substituted or unsubstituted with alkyl; aryl; and heteroaryl.
